(19) United States
(12) Patent Application Publication
(10) Pub. No.: US 2013/0333059 A1
(43) Pub. Date: Dec. 12, 2013

(54) HOX COMPOSITIONS AND METHODS

(71) Applicant: THE JOHNS HOPKINS UNIVERSITY, Baltimore, MD (US)

(72) Inventors: Saraswati Sukumar, Columbia, MD (US); Zhu Tao, Baltimore, MD (US); Xinyan Wu, Cockeysville, MD (US); Hexin Chen, Boyds, MD (US)

(73) Assignee: THE JOHNS HOPKINS UNIVERSITY, Baltimore, MD (US)

(21) Appl. No.: 13/627,743

(22) Filed: Sep. 26, 2012

Related U.S. Application Data
(63) Continuation of application No. 12/227,551, filed on Jun. 5, 2009, now abandoned.
(60) Provisional application No. 60/846,680, filed on Sep. 22, 2006.
(60) Provisional application No. 60/801,660, filed on May 19, 2006.

(51) Int. Cl. A61K 39/395; A61K 38/17; A61K 31/713; A61K 31/7088

(52) U.S. Cl. CPC A61K 39/39558; A61K 31/7088; A61K 38/1709; A61K 31/713
USPC 800/13; 435/375; 514/44 R; 514/1.1; 424/174.1; 514/44 A; 435/6.12; 435/7.1

(57) ABSTRACT

The present invention relates to compositions to treat HOXB7 related disorders. The invention also relates to methods treating HOXB7 related disorders. The invention further relates to kits for treating HOXB7 related disorders in a subject. The invention further relates to methods of identifying novel treatments for treating HOXB7 related disorders in a subject.

### CROSS-REFERENCE TO RELATED APPLICATIONS

This application is a Continuation application of U.S. application Ser. No. 12/227,551, filed Jun. 5, 2009, which is a U.S. National Phase Application pursuant to 35 U.S.C. §371, of PCT International Application Ser. No. PCT/US2007/012183, filed May 21, 2007, designating the United States and published in English on Nov. 29, 2007, as publication WO 2007/136857 A2, which claims the benefit of U.S. Provisional Application No. 60/846,680, filed Sep. 22, 2006, and U.S. Provisional Application No. 60/801,660, filed May 19, 2006, each of which is hereby incorporated by reference in their entirety.

### GOVERNMENT SUPPORT

This work was supported by the National Institutes of Health. The government may have certain rights in the invention.

### BACKGROUND

HOX genes, a subset of the homeobox gene family, are well conserved at the genomic level during evolution. In addition to their roles as master transcriptional factors in the regulation of embryonic development, their stringently regulated expression patterns in various tissues and organs in adulthood indicate fundamental roles in maintaining homeostasis. When HOXB7 is not properly regaled, diseases occur.

New methods are needed in the art to treat, prevent and ameliorate diseases mediated by HOXB7 over- and under-expression, e.g., DNA repair diseases, cancer, and chemotherapy resistance.

### BRIEF SUMMARY OF THE INVENTION

Provided herein are methods, treatments, screening assays, and animal models related to the HOXB7 protein, which is involved in DNA repair, tamoxifen resistance and cancer. The present invention provides novel compositions, methods, and kits to treat HOXB7 related disorders. The invention further provides methods of identifying novel treatments for treating HOXB7 related disorders in a subject.

In one aspect, provided herein are methods of modulating cellular processes, comprising modulating the functional level of a HOXB7 protein.

In one embodiment, increasing the functional HOXB7 protein level promotes DNA repair (increases efficiency of DNA repair).

In one aspect, provided herein are methods for the treatment and/or prophylaxis of a DNA repair condition in a mammal, comprising modulating the functional level of a HOXB7 protein in the mammal, wherein increasing functional levels of the HOXB7 protein level increases DNA repair activity of a cell.

In one embodiment, DNA repair is up-regulatable by HOXB7 protein over-expression.

In another embodiment, the increasing functional levels of the HOXB7 protein level is by up-regulation of a HOXB7 protein level and the up-regulation comprises introducing a nucleic acid molecule encoding a HOXB7 protein or functional equivalent, derivative or homologue thereof or the HOXB7 protein expression product or functional derivative, homologue, analogue, equivalent or mimetic thereof to the cell.

In one embodiment, the DNA repair condition comprises one or more of xeroderma pigmentosum (XP), Cockayne syndrome (CS), trichothiodystrophy (TTD), Fanconis anemia (FA), Bloom syndrome (BS), ataxia telangiectasia (AT), Fanconi's anemia, breast cancer or colon cancer.

In one embodiment, decreasing the functional levels of HOXB7 protein decreases epithelial-mesenchymal transition (or cancer progression or promotes migration and invasion characteristics) of a cell.

In one aspect, provided herein are methods for the treatment and/or prophylaxis of a condition characterized by aberrant or otherwise unwanted epithelial-mesenchymal transition, comprising modulating the functional level of a HOXB7 protein, wherein decreasing functional levels of HOXB7 protein decreases epithelial-mesenchymal transition.

In one aspect, provided herein are methods for the treatment and/or prophylaxis of a condition characterized by estrogen-response modulator resistance, comprising modulating the functional level of a HOXB7 protein in, wherein decreasing functional levels of HOXB7 protein.

In certain embodiments, the modulation is down-regulation of HOXB7 protein levels and the down-regulation comprises contacting the cell with a compound that functions as an antagonist to the HOXB7 protein expression product.

In one embodiment, the estrogen-response modulator resistance comprises tamoxifen resistance.

In another embodiment, the modulation comprises contacting the cell with a compound that modulates transcriptional and/or translational regulation of a HOXB7 gene.

In one embodiment, the compound comprises an siRNA targeting HOXB7.

In one embodiment, the siRNA comprises one or more of 5′-ATATCCAGCCTCAAGTTCG-3′ (SEQ ID NO: 1) or 5′-ACTTCTTGTGCGTTTGCTT-3′(SEQ ID NO: 2).

In one aspect, provided herein are uses of HOXB7, or homologues, derivatives or fragments thereof, for the manufacture of a medicament to treat HOXB7 related disorders.

In one embodiment, the HOXB7 related disorder comprises one or more of DNA repair disorder, cancer or estrogen-response modulator resistance

In one aspect, provided herein are pharmaceutical compositions comprising a pharmaceutically effective amount of a HOXB7 modulator effective to treat, prevent, ameliorate, reduce or alleviate a HOXB7 related disorder or symptoms thereof and a pharmaceutically acceptable excipient.

In one embodiment, the HOXB7 modulator is selected from one or more of a small molecule, RNAi molecule, an anti-HOXB7 antibody, an antigen-binding fragment of an anti-HOXB7 antibody, a polypeptide, a peptidomimetic, a nucleic acid encoding a peptide, or an organic molecule.

In one aspect, provided herein are methods to treat, prevent, ameliorate, reduce or alleviate a HOXB7 related disorder or symptoms thereof, comprising: administering to a subject in need thereof a composition comprising a pharmaceutically effective amount of a HOXB7 modulator.

In another embodiment, the HOXB7 modulator is one or more of a small molecule, an anti-HOXB7 antibody, an RNAi, an antigen-binding fragment of an anti-HOXB7 antibody, a polypeptide, a peptidomimetic, a nucleic acid encoding a peptide, or an organic molecule.

In one embodiment, the HOXB7 modulator is administered prophylactically to a subject at risk of being afflicted a HOXB7 related disorder.

In another embodiment, the composition further comprises a therapeutically effective amount of one or more of at least one anticonvulsant, non-narcotic analgesic, non-steroidal anti-inflammatory drug, antidepressant, glutamate receptor antagonist, nicotinic receptor antagonist, or local anesthetic.

In one embodiment, the composition is administered to the subject orally, intravenously, intrathecally or epidurally, intramuscularly, subcutaneously, perineurally, intradermally, topically or transcutaneously.

In one embodiment, the subject is a mammal

In another embodiment, the subject is a human.

In one embodiment, a HOXB7 related disorder or symptom thereof is indicated by alleviation of pain, progression of cancer, decreased cell proliferation, increased cell DNA repair efficiency, or an inhibition of cell proliferation.

The methods may further comprise obtaining the HOXB7 modulator.

In one aspect, provided herein are methods for identifying lead compounds for a pharmacological agent useful in the treatment of a HOXB7 related disorder comprising: contacting a cell expressing a HOXB7 protein with a test compound, and measuring HOXB7 expression, epithelial-mesenchymal transition, DNA repair activity, migration or invation activity, level of fibroblast growth factor (bFGF), levels of Ras, levels of RhoA, phosphorylation level of p44 and/or p42 mitogen activated protein kinase, localization of E-cadherin, localization of claudin-4, cell survival, plasmid end joining, clonogenic survival, expression of ER alpha, EGFR, HER2, Bcl-2, wound healing, invasion assays, and/or activation of Ras-MAP kinase pathways.

In one embodiment, an increase in DNA repair activity, an increase in plasmid end joining, an increase in clonogenic survival assays, an increase in cell survival assays or an increase in resistance to ionizing radiation indicates that the compound may be useful for treatment of a DNA repair disorder.

In another embodiment, a decrease in epithelial-mesenchymal transition, levels of Ras, levels of RhoA, phosphorlyation level of p44 and/or p42 mitogen activated protein kinase indicate that the composition may be useful in the treatment of cancer or epithelial-mesenchymal transition.

In one embodiment, an decrease in HOXB7 levels, expression of ER alpha, EGFR, HER2, and/or Bcl-2 indicates that the composition may be useful in the treatment of estrogen-response modulator resistance.

In one aspect, provided herein are methods for identifying lead compounds for a pharmacological agent useful in the treatment of a HOXB7 comprising: contacting a cell that does not express a functional amount of a HOXB7 protein with a test compound, and measuring one or more of HOXB7 expression or differentiation.

In another embodiment, EMT is measured by one or more of measuring protein or RNA expression, observing physical invasion or would healing markers, measuring protein or RNA levels of one or more of fibroblast growth factor (bFGF), levels of Ras, levels of RhoA, phosphorlyation level of p44 and/or p42 mitogen activated protein kinase, locilazation of E-cadherin, localization of claudin-4.

In another embodiment, the test compounds is one or more of a peptide, a small molecule, an antibody or fragment thereof, and nucleic acid or a library thereof.

In one aspect, provided herein are kits comprising: a) an HOXB7 modulator and a pharmaceutically acceptable carrier and b) instructions for use.

In one aspect, provided herein are transgenic non-human animals comprising an over-expressed HOXB7 protein or a fragment or variant thereof.

In one aspect, provided herein are uses of a transgenic animal according to claim 36, to test therapeutic agents.

In one embodiment, a HOXB7 modulator is administered to the subject orally, intramuscularly, intratumorally, stent, or intraperitoneally.

In one aspect, provided herein are methods for determining the therapeutic capacity of a HOXB7 modulator to reduce HOXB7 in a subject, comprising: performing an invasive surgical procedure on the subject; administering a HOXB7 inhibitor to the subject; and examining the subject for tumor growth.

In another embodiment, the invasive surgical procedure is a tumor removal.

In another embodiment, the subject is an animal model.

In one embodiment, the animal model is a tumor xenograft.

In one aspect, provided herein are methods for determining the therapeutic capacity of a HOXB7 inhibitor to reduce estrogen-response modulator resistance and/or prevent or inhibit tumor formation or progression in a subject, comprising: determining pre-treatment levels of HOXB7 in a subject; administering a therapeutically effective amount of a HOXB7 inhibitor to the subject; and determining a post-treatment level of HOXB7 in the subject.

In another embodiment, a decrease in the HOXB7 indicated that the HOXB7 inhibitor is efficacious.

In one embodiment, the pre-treatment and post-treatment levels of HOXB7 are determined in a diseased tissue.

In another embodiment, the diseased tissue is one or more of breast, skin, lung, heart, liver, tumor, or vasculature.

In one embodiment, the level of HOXB7 is determined by gene expression or protein expression.

In one aspect, provided herein are methods for determining the therapeutic capacity of a candidate HOXB7 modulator for treating a HOXB7 related disorder, comprising: contacting cells with a candidate composition, and determining effect of the candidate composition on one or more of HOXB7 expression, epithelial-mesenchymal transition, DNA repair activity, migration or invation activity, level of fibroblast growth factor (bFGF), levels of Ras, levels of RhoA, phosphorlyation level of p44 and/or p42 mitogen activated protein kinase, locilazation of E-cadherin, localization of claudin-4, cell survival, plasmid end joining, cologenic survival, expression of ER alpha, EGFR, HER2, Bcl-2, wound healing, invasion assays, and/or activation of Ras-MAP kinase pathways.

In one aspect, provided herein are methods for determining treatment of a subject suffering from breast cancer, comprising, determining the level of HOXB7 expression in a tumor of the subject and correlating HOXB7 over expression an indicator for the selection of fulvestrant as treatment.

In one embodiment, the methods of treating may further comprise identifying the subject as in need of treatment for a disease or condition involving HOXB7. In a related embodiment the identification compromises diagnosis of cancer, a DNA repair disease or estrogen-response modulator resistance.

In one embodiment, HOXB7 negative cells are correlated with not responding to E2, Tamoxifen or fulvestrant.

In one embodiment, the methods may further comprise determining the Her2 status of the tumor.

In one embodiment, Her2+ Hoxb7+ tumors are correlated with Trastuzumab susceptibility.

In one embodiment, treatment with Trastuzumab is followed by treatment with an anti-ER reagent.

In one embodiment, the anti-ER reagent comprises an aromatase inhibitor or Fulvestrant.

In one aspect, provided herein are methods of determining prognosis of breast cancer, comprising determining one or more of the HOXB7 status, ER status and Her2 status of a sample and correlating ER+ or Her2+ patient with high levels of Hoxb7 expression having a lower prognosis.

Other embodiments of the invention are disclosed infra.

### BRIEF DESCRIPTION OF THE DRAWINGS

FIG. 1 depicts HOXB7 mRNA levels in normal breast, primary breast carcinomas, and distant metastases. A, relative expression levels of HOXB7 mRNA in purified epithelial cells from human mammary epithelial cells (Normal), lymph node—positive invasive ductal carcinoma (IDC), and bone metastasis by microarray analysis. B, RT-PCR analysis of HOXB7 in normal human mammary epithelial cells (HMECs) and breast cancer cell lines. 36B4, a ribosomal protein mRNA that served as an internal loading control. C, the relative levels of HOXB7 mRNA as measured by quantitative real-time RT-PCR in nine purified mammary epithelial organoids from normal reduction mammoplasty samples, 31 primary breast carcinomas, and 19 breast metastasis to various organs. The two-tailed Student t test was applied for statistical analysis.

FIG. 2 depicts overexpression of HOXB7 in both MCF10A and MDCK cells induces EMT. A, phase-contrast photomicrographs of HOXB7 transfectants and control cell lines growing at low density on tissue culture plastic. Bar, 10 μm. B, Western blot analysis of Flag tagged HOXB7 (left) and HOXB7 (right) expression in MCF10A vector control (MCF10A-vec), four different clones of MCF10A-Flag-HOXB7 cell lines (10A-FB7D8, 10A-FB7C7, and 10A-FB7B8), MDCK vector control (MDCK-vec), and two different stable clones of MDCK-HOXB7 cell lines (MDCK-B7a and MDCK-B7c) and MDCK-HOXB7 pooled clones (MDCK-B7).

FIG. 3 shows that cells with high expression of HOXB7 express mesenchymal markers and show loss of epithelial markers. A, Western blot analysis of E-cadherin, claudin 1 (CLD1), claudin 4 (CLD4), claudin 7 (CLD7), α-smooth muscle actin (α-SMA), and vimentin in MCF10A-vec, pooled clones of MCF10A-FB7 (MCF10A-FB7), and three separate clones (10A-FB7-C2, 10A-FB7-C5, and 10A-FB7-C10), MDCK-vec and pooled clones of MDCK-B7 cells. B, MCF10A-vec and MCF10-FB7 cells were fixed and stained for F-actin with Alexa Fluor 488-phalloidin. MDCK-vec and MDCK-B7 cells were fixed and processed for immunofluorescence with antibodies recognizing E-cadherin (2), claudin 7 (3), claudin 1 (4), claudin 4 (5), vimentin (6), and α-smooth muscle actin (green; 7). 6 and 7, the same cells were costained with Alexa Fluor 555-phalloidin (red) and vimentin or α-smooth muscle actin (green).

FIG. 4 shows that overexpression of HOXB7 increases cell migration and invasion ability. A, wound healing assay with MDCK-vec and pooled MDCK-B7 cells. Microscopic observations were recorded 0, 6, 9, and 15 hours after scratching the cell surface. B, the cells were placed in a three-dimensional Matrigel invasion chamber and cells that invaded through Matrigel were fixed, stained with crystal violet, and counted. Arrow, a lamellipodia structure. Inset, higher magnification. C, number of cells that invaded through the Matrigel was counted in 10 fields under 20× objective lens. Bars, SD.

FIG. 5 depicts involvement of Ras-Raf-MAPK pathway in HOXB7-induced EMT. A, activity of Ras and RhoA was analyzed by GST pull-down assay with GST fusion proteins: GST-Raf Ras binding domain and GST-Rhotekin RhoA binding domain, respectively. Active Ras and RhoA, total Ras, and RhoA were determined by Western blot analysis. Phosphorylated MAPK and total MAPK were also determined by Western blot. B, inhibition of the activity of RAF and p42/44 MAPK by BAY 43-9006 (Raf-inh) and U0126 (MEK-inh), respectively, could block invasive ability of pooled MDCK-B7 cells. C and D, cotransfection of HOXB7 siRNAs SA1 and SA2 into MDA-MB-435 cells partially inhibited Ras and MAPK activity (C) and invasive ability of MDA-MB-435 (D) by reducing endogenous HOXB7 expression (C).

FIG. 6 depicts HOXB7 expression in MDCK cells confers tumorigenic potential in vivo. A, growth of pooled MDCK-B7 and MDCK-vec cells injected into the mammary gland fat pads of Swiss nu/nu mouse. B, RT-PCR verification of both HOXB7 and bFGF expressions in HOXB7-MDCK stable cell lines in culture and in xenograft tumors in nude mice. C, histopathology of xenograft MDCK-HOXB7 tumors. Tumor sections were stained with H&E (left). Arrows, invasion into the skeletal muscles (M) and tumor cells infiltrating into fat (T). Immunohistochemical analysis using monoclonal antibodies to the endothelial cell surface marker CD146 (middle) and cell proliferation marker Ki67 (right) are shown. Bar, 100 μm. D, treatment of MDCK-B7 cells with Su5402, a specific inhibitor of FGF receptor, could attenuate the active form Ras MDCK-B7 cells (top) and further reduce its invasive ability (bottom).

FIG. 7 depicts overexpression of HOXB7 can confer a transformed phenotype on MCF10A. A, MCF10A cells stably expressing HOXB7 or vector control cells were grown in RPMI supplemented with 1% or 10% FBS, and the cells were stained with crystal violet. Colorimetric measurements were done in triplicate for samples and each experiment was repeated thrice. B, Western analysis using anti-HOXB7 antibodies confirmed its expression in the stably transfected MCF10A-Fl-HOXB7 cells. The same blot reprobed with anti-β-actin antibodies provided loading controls. C, (1) MCF10A-vec cells remaining as 2 to 10 cell clusters grown in Matrigel; (2) MCF10A-Fl-HOXB7 cells formed large, anchorage-independent colonies when grown in Matrigel; (3) MCF10-vec cells formed acinar structures with hollow lumen; and (4) MCF10A-Fl-HOXB7 grew as irregular, solid colonies when grown on the surface of Matrigel. Phase contrast (magnification, ×20).

FIG. 8 depicts altered response of HOXB7-expressing MCF10A and SKBR3 cells to IR. A and B, clonogenic survival assays. Percentage of survival of MCF10A parental cells and MCF10A-Fl-HOXB7 or MCF10A-vec (A), or SKBR3 parental cells, SKBR3-HOXB7-YFP, SKBR3-YFP cells (B) irradiated at the indicated doses was calculated and compared with mock-irradiated (0 Gy) controls. C, G₁-type chromosomal aberrations after radiation treatment. The frequency of aberrations following irradiation with 3 Gy was calculated in parental SKBR3, SKBR3-HOXB7-YFP, and SKBR3-vec cells. D, G₂-type chromosomal aberrations after radiation treatment. Number of chromatid breaks and gaps in metaphases were scored for SKBR3-HOXB7-YFP cells and compared with those of SKBR3-YFP and parental SKBR3 controls. E, mitotic index after radiation treatment. Parental SKBR3, SKBR3-YFP, and SKBR3-HOXB7-YFP cells in exponential phase were irradiated with increasing doses of gamma radiation and then examined for the frequency of mitotic cells. Columns, mean value from three independent experiments; bars, SD. For each experiment, 200 metaphases were scored. F, HOXB7 stimulates DNA repair in vitro and in vivo. Plasmid end-joining assays were done. Nuclear extracts of SKBR3 cells expressing HOXB7-YFP or YFP alone were mixed with 0.25 μg of blunt-digested pCDNA3.0 in a plasmid end-joining reaction. Products were resolved on 0.7% ethidium bromide—stained agarose gels. Lane 1, DNA ladder; lane 2, undigested pCDNA3.0; lane 3, digested plasmid plus SKBR3-HOXB7-YFP nuclear extract; lane 4, digested plasmid plus SKBR3-YFP nuclear extract; lane 5, digested plasmid plus extraction buffer; lane 6, SKBR3-HOXB7-YFP nuclear extract minus plasmid. Band intensities were quantitated on Eagle Eye software. Columns, mean from two separate analyses; bars, SD.

FIG. 9 depicts identification and analysis of HOXB7 interacting proteins. A, affinity chromatography. GST-HOXB7 interacting proteins from SKBR3 cells (lanes 1-3, silver-stained gel; lane 4, Coomassie-stained PVDF membrane) or MCF10A cells (lanes 5-7, Coomassie-stained PVDF membrane). Lanes 1 and 7, proteins bound to GST alone (GST); lane 3, proteins bound to GST alone during the preclearing (GST p/c) step; lane 6, proteins bound to an unrelated control (GST-PRL3). *, positions of GST (lane 1 and 7) and GST-HOXB7 (lanes 2, 4, and 5). B, immunoblot confirmation of HOXB7-interacting proteins. Proteins which bound to GST-HOXB7 (lane 3) or control matrices (lanes 2 and 4) were eluted, separated by SDS-PAGE, and transferred to nitrocellulose and immunoblotted with antibodies to the DNA-PK_{cs}, PARP, Ku86, and Ku70. SKBR3 cell extracts (100 μg of total extract, 2% of input; lane 1) served as a positive control for proteins detected by immunoblot. C, coimmunoprecipitation of PARP, Ku80, and Ku70 with HOXB7-YFP in SKBR3 cells. SKBR3 cells were stably transfected with HOXB7-YFP (lanes 1, 4, and 7) or YFP alone as a vector control (lanes 2, 5, and 8) prior to immunoprecipitation with GFP antibodies and subsequent Western blot of precipitated proteins. Parental SKBR3 cells, which lack detectable HOXB7, were used as controls as well (lanes 3, 6, and 9). Lanes 1 to 3, protein levels in 100 μg of total cell extracts (5% of input); lanes 4 to 6, proteins that precipitated with HOXB7-YFP or controls that did not express HOXB7 (SKBR3-YFP and parental cells). Normal rabbit serum (NRS) was used to control for specificity (lanes 7-9). D, coimmunoprecipitation of HOXB7-YFP with DNA-PK_{cs} and Ku80 in SKBR3 cells. Complementary immunoprecipitations to those in FIG. 3C were done using SKBR3 cells transiently transfected with HOXB7-YFP (lanes 1, 4, and 7), YFP alone (lane 2, 5, and 8), or SKBR3 parental cells, which do not express HOXB7 (lanes 3, 6, and 9). Normal rabbit serum (NRS; lanes 10-12) was used as a nonspecific IgG control. E, DNA is not required for the interaction between HOXB7 and the DNA-PK complex. Extracts of MCF-7 cells were treated with ethidium bromide prior to coimmunoprecipitation with antibodies to Ku70 and Ku80, or p53 as a nonspecific IgG (NS IgG). Subsequent immunoblotting was done with the antibodies indicated (right). Top, effective blocking of the interaction between Ku70/80 and DNA-PK by using ethidium bromide depletion of DNA (positive control). Bottom, no effect of DNA depletion on the interactions between HOXB7 and Ku70 and Ku80.

FIG. 10 depicts analysis of HOXB7 complexes. A, Ku70/80 heterodimer formation is a prerequisite for HOXB7 binding. Fl-HOXB7 was transiently expressed in the CHO cells alone or with human Ku70 and/or human Ku80, coimmunoprecipitated with FLAG-antibodies, and immunoblotted with the antibodies indicated (right). Total protein lysates (100 μg; 5% of input). B, defining the region of HOXB7 that interacts with Ku70/80 proteins. Top, schematic representation of FLAG-tagged HOXB7 (FLAG-HOXB7), and deletion constructs. Locations of the FLAG tag (Fl), homeodomain (H), and deletions of the third helix (Δh3) of the homeodomain, and of the glutamic acid-rich tail (ΔGlu). Bottom, Fl-HOXB7, HOXB7-Δh3, and HOXB7-ΔGlu were transfected into SKBR3 cells, and cell lysates were subjected to coimmunoprecipitation with the anti-FLAG antibody, and then immunoblotted with the antibodies indicated (left).

FIG. 11 depicts HOXB7 stimulates DNA-PK activity and the helix-3 domain is indispensable for HOXB7-mediated enhancement of cell survival and NHEJ. A, DNA-PK activity is enhanced in HOXB7-expressing cells. DNA-PK activity was measured in DNA-depleted whole cell extracts prepared from cells transiently transfected with either the empty vector or with plasmids expressing Fl-HOXB7 or HOXB7-Δh3. Columns, mean values of duplicate samples from three experiments; bars, SD. B, clonogenic survival assays. Survival of SKBR3 cells transiently transfected with Fl-HOXB7, HOXB7-Δh3, or the empty vector after irradiation were compared with mock-irradiated (0 Gy) controls. C, DNA DSB repair. Cells were irradiated with 50 Gy and lysed at different periods after irradiation. Control cells are repair-deficient ataxia telangiectasia cells (AT), GM5823. Unrepaired DNA breaks were measured under nondenaturing conditions. Points, means from three independent experiments; bars, SD.

FIG. 12 depicts knockdown of endogenous HOXB7 reduces DNA repair efficiency. A and B, clonogenicity after exposure to radiation of MCF-7 and MDA-MB-468 cells transfected with HOXB7-specific siRNA. Clonogenic survival assays of MCF-7 (A) or MDA-MB-468 (B) cells stably transfected with plasmids expressing either scrambled sequence siRNA (Scr.-siRNA) or HOXB7-specific siRNA (HOXB7-siRNA) were done. Survival was calculated on day 14 for MCF-7 and on day 10 for MDA-MB-468 relative to mock-irradiated (0 Gy) controls. C, analysis of chromosome damage and repair in MDA-MB-435 cells with or without reduced levels of HOXB7. Cells with HOXB7 knockdown (HOXB7-siRNA) showed significant differences in chromosomal aberration frequencies per metaphase compared with control cells (Scr.-siRNA). D, knockdown of HOXB7 reduces level of DNA DSB repair in MDA-MB-435. Cells with and without reduced levels of HOXB7 by transfection of HOXB7-specific (HOXB7-siRNA) or scrambled siRNA (Scr.-siRNA) along with parental cells were irradiated with 50 Gy and unrepaired DNA breaks were measured by PFGE. Points, means from three independent experiments; bars, SD.

FIG. 13 depicts HOXB7 overexpression promotes tamoxifen resistance. (a) Western blot analysis of the expression of HOXB7 in MCF-7-vec and MCF-7-B7 cells and growth curve of MCF-7-vec and MCF-7-B7 cells grown in monolayer culture. (b) Soft agar colony formation by MCF-7-vec and MCF-7-B7 cells. (c) Tumor growth curves of MCF-7-vec and MCF-7-B7 cells in implanted s.c. in athymic mice in presence of an exogenous slow release, estrogen implant. (d) Histologic appearance of the tumors visualized with hematoxylin and eosin staining. (e) Magnetic resonance imaging of MCF-7-B7 cells, MCF-7 parental cells and MCF-7-vec cells (f) Magnetic resonance imaging of vascularity of MCF-7-vec and MCF-7-HOXB7 cells. (g) Tumor growth curve of MCF-7-vec cells and MCF-7-B7 implanted s.c. in athymic mice in absence of exogenous estrogen supplementation. Soft agar colony formation by MCF-7-vec and MCF-7-B7 cells treated with either vehicle, tamoxifen (1 μM) or estrogen (10 nM) in complete medium (h) or estrogen-deprived medium (j). MCF-7-vec cells or MCF-7-B7 cells were transfected with ERE-tk-Luc reporter plasmid and β-Gal expression plasmid in presence of a combination of either vehicle, 100 nM tamoxifen, or 10 nM E2 as indicated for 24 h, reporter activity measured and normalized by β-Gal activity (i).

FIG. 14 depicts HOXB7 expression promotes EGFR/HER2 and ERα signaling. (a) Immunoblot analysis of both active form and total EGFR or HER2 expression levels in MCF-10A-B7 or MCF-7-B7 cells and their vector (vec) controls. (b) MCF-7 cells were transfected with pcDNA3-Flag-HOXB7 plasmid and chromatin immunoprecipitation was performed by immunoprecipitating with either anti-Flag M2 antibody or control IgG. Schematic representation of the location of the targeted DNA fragment used for ChIP assay. (c) Luciferase activity of deletion constructs of the EGFR promoter region to map minimal region necessary for activation by HOXB7. (d) Western blot analysis of expression levels of phosphorylated p44/42 or Akt in MCF-10A-B7 or MCF-7-B7 cells. (e) RT-PCR analysis of mRNA expression levels of TGFα, HB-EGF and Amphiregulin in HOXB7 expressing MCF-10A or MCF-7 cells and their vector controls. Immunoblot analysis of (0 TGFα or HB-EGF in HOXB7 expressing MCF-10A cells and the vector control cells treated with the 0.1 μM AG1478 or vehicle for 24 hours. (g) ERα, ERβ, P-ERα (ser118) or P-ERα (ser167) expression in MCF-7-vec and MCF-7-B7 cells. (h) ER-downstream genes, PR-B, c-Myc, Cyclin D1, Bcl-2 in MCF-7-vec and MCF-7-B7 cells. (i) EGFR, HER2, ER and Bcl-2 in MCF-7-vec and MCF-7-B7 cells transfected with plasmid expressing either scrambled sequence siRNA or HOXB7-specific siRNAs, S3, S4, or both.

FIG. 15 shows that HOXB7 overexpression in MCF-7 cells converts tamoxifen into an agonist Immunoblot analysis of (a) P-EGFR, P-HER2, P-MAPK, P-ERα (ser118), Cyclin D1 or Bcl-2 expression in MCF-7-vec and MCF-7-B7 cells treated with either vehicle, 10 nM estrogen or 1 μM of tamoxifen in estrogen deprived medium for 48 h. (b) P-EGFR, P-HER2, P-MAPK, and P-ERα (ser118) expression in MCF-7-vec and MCF-7-B7 cells treated with either vehicle or 1 μM of gefitinib for 24 h. (c) Amphiregulin or TGFα expression in MCF-7-vec and MCF-7-B7 cells treated with either vehicle or 1 μM of fulvestrant for 24 h. (d) Amphiregulin or TGFα expression in MCF-7-vec and MCF-7-B7 cells transfected with either scrambled sequence siRNA or ERα-specific siRNA. (e) Soft agar colony formation by MCF-7-vec and MCF-7-B7 cells treated with vehicle alone, 1 μM tamoxifen alone, or 1 μM tamoxifen in combination with 1 μM gefitinib as indicated.

FIG. 16 depicts targeting of HOXB7 reverses tamoxifen resistance. (a) Soft agar colony formation by MCF-7-vec and MCF-7-B7 cells transfected with either scrambled sequence siRNA or HOXB7-specific siRNA in presence of either vehicle or 1 μM tamoxifen. (b) Immunoblot analysis of EGFR, HER2, ERα and P-MAPK expression in BT474 cells transfected with either scrambled sequence siRNA or HOXB7-specific siRNA. (c) Soft agar colony formation by (1) BT474 cells treated with 1 μM gefitinib alone, or a combination of 1 μM gefitinib and 1 μM tamoxifen, and (2) BT474 cells transfected with either scrambled sequence siRNA or HOXB7-specific siRNA in presence of either vehicle, or 1 μM tamoxifen.

FIG. 17 shows that HOXB7 promotes acquired tamoxifen resistance. Immunoblot analysis of (a) EGFR, HER2, ERα, and HOXB7 expression in MCF-7 cells treated long-term with either vehicle or 1 μM tamoxifen (TMR1) or 0.1 μM tamoxifen (TMR2). (b) EGFR, HER2, ERα, and HOXB7 expression in MCF-7 cells treated with either vehicle for 6 months or 0.1 μM tamoxifen for 2, 4, and 6 months. (c) EGFR, HER2, ERα, and HOXB7 expression in MCF-7-TMR cells transfected with either scrambled sequence siRNA or HOXB7-specific siRNA. (d) Oscillative expression of EGFR, HER2, and HOXB7 in T47D cells upon tamoxifen treatment for 72 h. (e) RT-PCR analysis of HOXB7 expression resulting from tamoxifen exposure is dependent on ERα pathway. (f) CHIP analysis of T47D cells treated with either vehicle, 10 nM E2, or 1 μM tamoxifen for 45 min; IP using anti-ERα or anti-ERβ antibody. Precipitated DNA was analyzed by PCR using primers specific for HOXB7 promoter region Immunoblot analysis of (g) Tumor growth curve of MCF-7-vec cells and MCF-7-B7 cells implanted s.c. in athymic Swiss female mice and treated with either vehicle, tamoxifen (83.3 μg/day), or fulvestrant (10 mg/week) in the absence of an exogenous estrogen supplement. EGFR, HER2, ERα, and HOXB7 expression in MCF-7-TMR cells treated with either vehicle or 1 μM fulvestrant for 24 h. (h) EGFR, HER2, ERα and HOXB7 expression in 2 different anti-estrogen resistance models (MCF-7-LTED, 7-TAMLT). (i) Summary comparison of expression levels of EGFR, HER2, ERα and HOXB7 in 4 different anti-estrogen resistance models. (j) Schematic model of the functional role of HOXB7 in tamoxifen resistance.

FIG. 18 depicts MCF-10A cells or MCF-7 cells were stably transfected with either the empty vector (MCF-10A-vec, MCF-7-vec) or the vector containing Flag tagged HOXB7 cDNA (MCF-10A-B7, MCF-7-B7). The level of HOXB7 mRNA was determined by RT-PCR (a). (b) Phase-contrast images of MCF-10A cells expressing vector or HOXB7 cultured to confluence. (c) Monolayer growth curve of MCF-10A-vec or MCF-10A-B7 cells in DMEM/F-12 medium supplemented with either 5% horse serum or 0.1% horse serum. (d) Monolayer growth curve of MCF-7-vec cells and MCF-7-B7 cells in estrogen deprived medium. (e) Effect of HOXB7 overexpression on the protection from tamoxifen (2 μM) treatment promoted apoptosis. Apoptotic cell death was determined by fluorescent microscopic analysis of cell DNA staining patterns with Hoechst 33258 as described in Materials and Methods. Bars, ±SD in triplicate assays. (f) Tumor growth curve of MCF-7-vec cells implanted in the athymic female mice s.c. and treated either vehicle or 83.3 μg/day of tamoxifen with exogenous estrogen supplement.

FIG. 19 depicts (a) Western blot analysis of expression of EGFR, HER2 and HOXB7 in MCF-7 and HBL-100 cells transiently transfected with either empty vector or HOXB7 expression plasmid. (b) Semi-quantitative RT-PCR analysis of EGFR expression in MCF-10A-B7 and MCF-7-B7 cells and their cognate control cells. (c) MCF-7-vec or MCF-7-B7 cells were transiently co-transfected with β-Galactosidase expression plasmid and ERE-tk-Luc reporter plasmid in phenol-red free RPMI medium with 5% harcoal stripped serum for 24 hours in presence of indicated concentration of estrogen. The relative luciferase activities presented were normalized by β-galactosidase activity. (d) MCF-7-vec or MCF-7-B7 cells were transiently co-transfected with β-Galactosidase expression plasmid, reporter plasmid pFR-Luc and fusion trans-activator plasmid (pFA-c-Fos or pFA-c-Jun as indicated). The relative luciferase activities presented were normalized by protein concentrations as well as β-galactosidase activity (mean±S.D., n=3).

FIG. 20 depicts (a) Verification of the efficacy of HOXB7 specific siRNA by use of RT-PCR and Western blot analysis. (b) Soft agar colony formation by MCF-7-vector and MCF-7-HOXB7 cells transfected with the plasmid expressing either scrambled sequence siRNA or HOXB7 specific siRNA (either S3, S4, or S3+S4) for 24 h. (c) Expression levels of EGFR, HER2, P-MAPK, P-Akt and HOXB& in MDA-MB-468 cells transfected with either scrambled sequence siRNA or HOXB7 specific siRNA. (d) Expression levels of EGFR, HER2, P-MAPK, HOXB7, and Bcl-2 in MDA-MB-435 cells transfected with either scrambled sequence siRNA or HOXB7 specific siRNA. Bars represent mean±S.D. *, p<0.05.

FIG. 21 depicts (a) Soft agar colony formation by MCF-7-Sen and MCF-7-TMR cells treated with either vehicle or 100 nM tamoxifen. (b) MCF-7-Sen cells or MCF-7-TMR cells were transfected with ERE-tk-Luc reporter plasmid and β-Gal expression plasmid in presence of a combination of either vehicle, 100 nM tamoxifen, or 10 nM E2 as indicated for 24 h. Reporter activity is normalized by β-Gal activity; (c) Soft agar colony formation by MCF-7-HOXB7 and MCF-7-TMR cells treated with either vehicle or 1 μM Fulvestrant. Bars represent mean±S.D. *, p<0.05.

FIG. 22 depicts expression status of HOXB7 was examined in breast cancer cell lines.

FIG. 23 depicts a MMTV-HOXB7 transgenic mouse model.

FIG. 24 depicts HOXB7 mice did not develop mammary tumors.

FIG. 25 depicts stained whole-mounts of mammary glands.

FIG. 26 depicts a model wherein Her2-induced tumorigenesis was artificially divided into two phases: tumor onset and tumor progression.

FIG. 27 depicts sacrificed the mice at 10 weeks after we first palpated the tumor.

FIG. 28 depicts Ki67-staining analysis. As shown, about 30% of her2-tumor cells are ki67-positive while about 80% of Hoxb7 and Her2 double positive cells are ki67-positive. The figure to the right is the summary of ki67-staining analysis of 15 pairs of samples. These results strongly suggested that overexpression of Hoxb7 in tumor cells promotes cellular proliferation.

FIG. 29 depicts in vitro data showed that Hoxb7 induces EMT.

FIG. 30 depicts the molecular basis for the dual role of Hoxb7 in Her2-induced tumorigeneis-delay tumor onset and promote tumor progression was examined.

FIG. 31 depicts expression of Her2 in both Hoxb7+/− tumor samples.

FIG. 32 depicts cells labeled using SILAC methods.

FIG. 33 depicts an MS spectrum of Her2-derived peptide (SEQ ID NO: 24).

FIG. 34 depicts using SILAC, to identify and quantiate 395 proteins.

FIG. 35 depicts the results of a microarray analysis used to identify the Hoxb7 target genes.

FIG. 36 depicts the expression levels of ER examined in both Hoxb7-negative and positive tumor cells.

FIG. 37 depicts the results of a statistical analysis using published microarray data.

### DETAILED DESCRIPTION

This invention is based, in part, on the discovery of that the HOXB7 protein is involved in DNA repair, tamoxifen resistance and in cancer. The present invention provides novel compositions, methods, and kits to treat HOXB7 related disorders. The invention further provides methods of identifying novel treatments for treating HOXB7 related disorders in a subject.

### DEFINITIONS

“Agonist,” as used herein refers to a compound or composition capable of combining with (e.g., binding to, interacting with) receptors to initiate pharmacological actions.

Pharmaceutically acceptable refers to, for example, compounds, materials, compositions, and/or dosage forms which are suitable for use in contact with the tissues of human beings and animals without excessive toxicity, irritation, allergic response, or other problems or complications, commensurate with a reasonable benefit/risk ratio.

Pharmaceutically acceptable salts refer to, for example, derivatives of the disclosed compounds wherein the compounds are modified by making at least one acid or base salt thereof, and includes inorganic and organic salts.

An effective antagonistic amount of HOXB7 modulator refers to an amount that effectively attenuates (e.g. blocks, inhibits, prevents, or competes with) the activity of the HOXB7 protein.

A therapeutically effective amount of a HOXB7 composition refers to an amount that elicits alleviation or lessening of at least one symptom of pain upon administration to a subject in need thereof.

Potency refer, for example, to the strength of a composition or treatment in producing desired effects, for example, HOXB7 expression and/or the alleviation of, for example, symptoms described infra. Potency also may refer to the effectiveness or efficacy of a composition in eliciting desired effects, for example, initiation of HOXB7 expression or exit from HOXB7 expression. Enhanced potency, for example, refers to the lowering of a dose in achieving desired effects or to an increased therapeutic benefit including that not previously seen, for example, where the increased therapeutic benefit is eliciting desired effects such as HOXB7 expression from oral administration, oral formulation or oral dosage form. In therapeutics, for example, potency may refer to the relative pharmacological activity of a compound or a composition.

The following terms encompass polypeptides that are identified in Genbank by the following designations, DQ976548; NM_{—}010460; BC015345; AF287967; CAC93674; ABM46823; and AAH97639, which are hereby incorporated by reference, as well as polynucleotides and polypeptides that are at least about 70% identical to the polynucleotides and polypeptides identified in Genbank by these designations as described infra. In alternative embodiments, these terms encompass polypeptides identified in Genbank by these designations and polypeptides sharing at least about 80, 90, 95, 96, 97, 98, or 99% identity.

A “HOXB7 modulator” is either an inhibitor or an enhancer of a HOXB7 protein or a HOXB7 protein. A “non-selective” HOXB7 protein family modulator is an agent that modulates other HOXB7 proteins at the concentrations typically employed for HOXB7 modulation. A “selective” HOXB7 modulator significantly modulates one or more of the normal functions of an HOXB7 protein at a concentration at which other HOXB7 proteins are not significantly modulated. A modulator “acts directly on” a HOXB7 protein when the modulator binds to the HOXB7 protein. A modulator “acts indirectly on a HOXB7 protein” when the modulator binds to a molecule other than the HOXB7 protein, which binding results in modulation of the protein.

A “modulator of a HOXB7 protein” is an agent that can affect: (1) the expression; mRNA stability; or protein trafficking, modification (e.g., phosphorylation), protein stability, the functional level of HOXB7, or degradation of a HOXB7 protein, or (2) one or more of the normal functions of a HOXB7 protein. An modulator of a HOXB7 protein can be non-selective or selective.

An “enhancer of a HOXB7 protein” is an agent that increases by any mechanism as compared to that observed in the absence (or presence of a smaller amount) of the agent. An enhancer of a HOXB7 protein can affect: (1) the expression; mRNA stability; or protein trafficking, modification (e.g., phosphorylation), increases the functional HOXB7 protein level, or degradation of a HOXB7 protein; or (2) one or more of the normal functions of a HOXB7 protein. An enhancer of an HOXB7 protein can be non-selective or selective.

In one embodiment the present invention is directed to up regulating the functional level of HOXB7 to introducing HOXB7 expression to a population of cells. However, it should nevertheless be understood that there are circumstances in which it is desirable to down regulate the functional level of HOXB7 to obviate the expression of these characteristics or to end aberrant HOXB7 expression.

For example, one may seek to up regulate the functional level of HOXB7 in the context of a defined population of cells for a period of time sufficient to achieve a particular objective, e.g., increase DNA repair mechanisms of a cell. However, once that objective has been achieved one would likely seek to down regulate the intracellular functional level of HOXB7, to the extent that it is not transient, such that it is no longer over-expressed and the subject cells. In another example, one may identify certain disease conditions which are characterized by an over-expression of the functional level of HOXB7, e.g., cancer. In such a situation, one may observe uncontrolled cell proliferation which could lead to tumor formation. Where such a situation exists, one may seek to down regulate the functional level of HOXB7 to end aberrant cell growth (e.g, tumor growth). Accordingly, down-regulation of cell HOXB7 levels would be desirable as a therapeutic treatment. The present invention should therefore be understood to be directed to up regulating the HOXB7 functional level in order to introduce unique phenotypic properties to the population of cells and down-regulating a naturally or non-naturally induced state of HOXB7 over-expression to treat other unique phenotypic properties (e.g., cancer and/or estrogen-response modulator resistance (e.g., tamoxifen resistance)).

As detailed above, reference to “modulating” HOXB7 functional levels is a reference to either up regulating or down regulating these levels. Such modulation may be achieved by any suitable means and include, for example: (i) modulating absolute levels of the active or inactive forms of HOXB7 (for example increasing or decreasing intracellular HOXB7 concentrations) such that either more or less HOXB7 is available for activation and/or to interact with its downstream targets. (ii) Agonising or antagonising HOXB7 such that the functional effectiveness of any given HOXB7 molecule is either increased or decreased. For example, increasing the half life of HOXB7 may achieve an increase in the overall level of HOXB7 activity without actually necessitating an increase in the absolute intracellular concentration of HOXB7. Similarly, the partial antagonism of HOXB7, for example by coupling HOXB7 to a molecule that introduces some steric hindrance in relation to the binding of HOXB7 to its downstream targets, may act to reduce, although not necessarily eliminate, the effectiveness of HOXB7 signaling. Accordingly, this may provide a means of down-regulating HOXB7 functioning without necessarily down-regulating absolute concentrations of HOXB7.

In terms of achieving the up or down-regulation of HOXB7 functioning, methods and techniques for achieving this objective would be well known to the person of skill in the art and include, for example: (i) introducing into a cell a nucleic acid molecule encoding HOXB7 or functional equivalent, derivative or analogue thereof in order to up-regulate the capacity of The cell to express HOXB7. (ii) Introducing into a cell a proteinaceous or non-proteinaceous molecule which modulates transcriptional and/or translational regulation of a gene, wherein this gene may be a HOXB7 gene or functional portion thereof or some other gene which directly or indirectly modulates the expression of the HOXB7 gene. (iii) introducing into a cell the HOXB7 expression product (in either active or inactive form) or a functional derivative, homologue, analogue, equivalent or mimetic thereof. (iv) introducing a proteinaceous or non-proteinaceous molecule which functions as an antagonist to the HOXB7 expression product. (v) introducing a proteinaceous or non-proteinaceous molecule which functions as an agonist of the HOXB7 expression product.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

A “test agent” is any agent that can be screened in the prescreening or screening assays of the invention. The test agent can be any suitable composition, including a small molecule, peptide, or polypeptide.

The term “therapy,” as used herein, encompasses the treatment of an existing condition as well as preventative treatment (i.e., prophylaxis). Accordingly, “therapeutic” effects and applications include prophylactic effects and applications, respectively.

A used herein, the term “high risk” refers to an elevated risk as compared to that of an appropriate matched (e.g., for age, sex, etc.) control population.

“Nucleic acids,” as used herein, refers to nucleic acids that are isolated a natural source; prepared in vitro, using techniques such as PCR amplification or chemical synthesis; prepared in vivo, e.g., via recombinant DNA technology; or by any appropriate method. Nucleic acids may be of any shape (linear, circular, etc.) or topology (single-stranded, double-stranded, supercoiled, etc.). The term “nucleic acids” also includes without limitation nucleic acid derivatives such as peptide nucleic acids (PNA's) and polypeptide-nucleic acid conjugates; nucleic acids having at least one chemically modified sugar residue, backbone, internucleotide linkage, base, nucleoside, or nucleotide analog; as well as nucleic acids having chemically modified 5′ or 3′ ends; and nucleic acids having two or more of such modifications. Not all linkages in a nucleic acid need to be identical.

In general, the oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). However, single-stranded DNA is preferred, as DNA is often less labile than RNA. Similarly, chemical modifications that enhance an aptamer's specificity or stability are preferred.

Chemical modifications that may be incorporated into nucleic acids include, with neither limitation nor exclusivity, base modifications, sugar modifications, and backbone modifications. Base modifications: The base residues in aptamers may be other than naturally occurring bases (e.g., A, G, C, T, U, 5MC, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine (5MC), N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenylade-nine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in aptamers. Sugar modifications: The sugar residues in aptamers may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3-(and 4-) hydroxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Om), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi nlm nih.go-v/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

A “radioligand binding assay” is an assay in which a biological sample (e.g., cell, cell lysate, tissue, etc.) containing a receptor is contacted with a radioactively labeled ligand for the receptor under conditions suitable for specific binding between the receptor and ligand, unbound ligand is removed, and receptor binding is determined by detecting bound radioactivity.

As used herein, an “antibody” refers to a protein consisting of one or more polypeptides substantially encoded by immunoglobulin genes or fragments of immunoglobulin genes. Immunoglobulin genes include, for example, the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term antibody, as used herein also includes antibody fragments either produced by the modification of whole antibodies or synthesized de novo using recombinant DNA methodologies, see for example, Fundamental Immunology, W. E. Paul, ed., Raven Press, N.Y. (1993), for a more detailed description of other antibody fragments. While various antibody fragments are defined in terms of the digestion of an intact antibody, one of skill will appreciate that such Fab′ fragments may be synthesized de novo either chemically or by utilizing recombinant DNA methodology. Antibodies also include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, F light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778).

The phrases “an effective amount” and “an amount sufficient to” refer to amounts of a biologically active agent that produce an intended biological activity.

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH 7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“HOXB7” or “HOXB7 protein” or HOXB7 protein family” refer to a protein or family of homeobox domain proteins.

It has also been determined that expressing or over-expressing HOXB7 in a cell can result in improved DNA repair. Accordingly, reference to “modulating” HOXB7 expression of a cell “relative to” normal cell characteristics should be understood to include the over-expression of HOXB7 levels results in an increased efficiency of DNA repair and longer cell survival and increased resistance to ionizing radiation. Without limiting the present invention in any way, examples of characteristics which may be induced in cells over-expressing HOXB7 levels include, for example:

1) improved proliferative characteristics both in terms of an increased rate/extent of proliferation and the requirement for only minimal environmental/cell culture conditions under which proliferation can occur (herein referred to as “enhanced proliferation”);

2) improved cell viability, which may occur either at the level of down regulating apoptosis or preventing or otherwise induced cell death. For example, cell survival under conditions of stress (such as the removal of tissue culture supplements in the in vitro environment) is facilitated as is the down regulation of apoptosis which would normally occur in the absence of the anti-apoptotic signals which are provided as a result of integrin receptor engagement during matrix attachment and cell spreading. This is particularly relevant, for example, where in vitro cell culture populations are required to be maintained in suspension (herein referred to as “enhanced viability”); or

3) increased efficiency of DNA repair mechanisms.

It has also been determined that decreasing the levels of HOXB7 in a cell can result in improved decreases in tumor formation and progression. Accordingly, reference to “modulating” HOXB7 expression of a cell “relative to” normal cell characteristics should be understood to include the decreasing levels results in lower tumor formation and progression.

As used herein, “functional level” of HOXB7 should be understood as a reference to the level of HOXB7 activity which is present in any given cell as opposed to the concentration of HOXB7. Although an increase in the concentration of HOXB7 will generally correlate to an increase in the level of HOXB7 functional activity which is observed in a cell, the person skilled in the art would also understand that increases in the level of activity can be achieved by means other than merely increasing absolute intracellular HOXB7 concentrations. For example, one might utilize forms of HOXB7 which exhibit an increased half-life or otherwise exhibit enhanced activity. Reference to “over-expressing” the subject HOXB7 level should therefore be understood as a reference to up regulating intracellular HOXB7 to an effective functional level which is greater than that expressed under the normal physiological conditions for a given cell prior to HOXB7 expression or to the up-regulation of HOXB7 levels to any level of functionality but where that up-regulation event is one which is artificially effected rather than being an increase which has occurred in the subject cell due to the effects of naturally occurring physiology prior to HOXB7 expression. Accordingly, this latter form of up-regulation may correlate to up-regulating HOXB7 to levels which fall within the normal physiological range but which are higher than pre-stimulation or pre-HOXB7 expression levels. The mechanism by which up-regulation is achieved may be artificial mechanism that seek to mimic a physiological pathway—for example introducing a hormone or other stimulatory molecule, e.g., retinoic acid (RA). Accordingly, the term “expressing” is not intended to be limited to the notion of HOXB7 gene transcription and translation. Rather, it is a reference to an outcome, being the establishment of a higher and effective functional level of HOXB7 than is found under normal physiological conditions in a cell at a particular point in time (e.g., it includes non-naturally occurring increases in HOXB7 level, even where those increases may fall within the normal physiological range which one might observe). Reference to the subject functional level being an “effective” level should be understood as a level of over-expression which achieves the modulation of HOXB7 expression of a cell relative to a normal cell.

Determining the specific functional level (e.g., “effective” level) to which the HOXB7 should be up or down-regulated in order to achieve the desired phenotypic change for any given cell type is a matter of routine procedure. The person of skill in the art would be familiar with methods of determining such a level. “Modulating cellular HOXB7 expression,” as used herein includes, any up or down-regulation of HOXB7 expression.

Methods of Treating

In one aspect, provided herein are methods to treat, prevent, ameliorate, reduce or alleviate a HOXB7 related disease or symptoms thereof, comprising: administering to a subject in need thereof a composition comprising a pharmaceutically effective amount of a HOXB7 modulator.

In one aspect, it is efficacious to decrease the level of functional HOXB7 in a cell and in other aspects it is efficacious to increase the level of functional HOXB7 in a cell. Thus, both antagonists and agnosits are proposed as therapeutic methods.

An “effective amount” includes, for example, an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

In one embodiment, the composition is administered to the subject orally, intravenously, intrathecally or epidurally, intramuscularly, subcutaneously, perineurally, intradermally, topically or transcutaneously.

Subjects include mammals, e.g., humans, cows, pigs, horses, squirrels, primates, dogs, cats, rabbits, goats, etc.

“Obtaining the HOXB7 modulator,” as used herein refers to making or buying the modulator.

In one embodiment, a HOXB7 related disorder or symptom thereof is indicated by alleviation of pain, progression of degenerative disease, progression of cancer, decreased cell proliferation, increased efficiency of DNA repair and decreased resistance to estrogen-response modulators (e.g., tamoxifen).

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered to include reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, for example, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in herein may be co-administered with one or more other compounds or molecules. By “co-administered” is meant simultaneous or sequential administration in the same formulation or in two different formulations via the same or different routes or sequential administration by the same or different routes. For example, the subject HOXB7 agonist or antagonist may be administered together with an agonistic agent in order to enhance its effects. Alternatively, in the case of organ tissue transplantation, the HOXB7 agonist or antagonist may be administered together with immunosuppressive drugs. By “sequential” administration is meant a time difference of from seconds, minutes, hours or days between the administration of the two types of molecules. These molecules may be administered in any order. In another embodiment, the composition further comprises a therapeutically effective amount of one or more of at least one anticonvulsant, non-narcotic analgesic, non-steroidal anti-inflammatory drug, antidepressant, glutamate receptor antagonist, nicotinic receptor antagonist, or local anesthetic.

Another aspect of the present invention relates to the use of an agent capable of modulating the functional level of HOXB7 in the manufacture of a medicament for the modulation of cell HOXB7 expression in a mammal wherein inducing over-expression of the HOXB7 level modulates cell HOXB7 expression of the cells.

In another aspect, the present invention relates to the use of HOXB7 or a nucleic acid encoding HOXB7 in the manufacture of a medicament for the modulation of cell HOXB7 expression in a mammal wherein inducing over-expression of the HOXB7 level modulates cell HOXB7 expression of the cells.

“Aberrant or otherwise unwanted cellular HOXB7 expression” refers, for example, to conditions in a mammal, wherein HOXB7 expression desired and not occurring or vice verse. Aberrant HOXB7 expression may happen, for example, one or more of a neuronal cell, a pancreatic cell, a lung cell, bone tissue cell, a spleen cell, heart cell, kidney cell, a testis cell, or an intestinal tract cell. The aberrant HOXB7 expression may lead, for example, to one or more of the following conditions: cancer, degenerative diseases (ALS, Alzheimer's disease), infertility, pulmonary disease, tissue engineering, nerve damage, gastrointestinal disease, pain (chronic, neuropathic, acute), trauma, migraine, neurological disorders (anxiety, stroke, psychoses, schizophrenia, depression, epilepsy), cardiovascular conditions (hypertension and cardiac arrhythmias), or diabetes. The HOXB7 expression is up-regulatable by HOXB7 protein over-expression and down-regulatable by reducing the functional level of HOXB7 protein level.

The modulation may be the up-regulation of a HOXB7 protein level and the up-regulation for example by the introduction a nucleic acid molecule encoding a HOXB7 protein or functional equivalent, derivative or homologue thereof or the HOXB7 protein expression product or functional derivative, homologue, analogue, equivalent or mimetic thereof to the cell. The modulation may also be by contacting the cell with a compound that modulates transcriptional and/or translational regulation of a HOXB7 gene. The modulation may also be by contacting the cell with a compound that functions as an agonist of the HOXB7 protein expression product.

In the one embodiment, the modulation is down-regulation of HOXB7 protein levels and the down-regulation may be done by contacting the cell with a compound that functions as an antagonist to the HOXB7 protein expression product.

In either up- or down-regulation, the modulation of HOXB7 expression may be in vivo or in vitro.

In one aspect, provided herein are methods of converting a stem cell into a ventral neuron which comprises introducing into the stem cell a nucleic acid which expresses homeodomain transcription factor Nkx6.1 protein in the stem cell so as to thereby convert the stem cell into the ventral neuron.

In one aspect, provided herein are methods of converting a motor neuron progenitor into a post-mitotic neuron comprising introducing a nucleic acid expressing a HOXB7 protein into the motor neuron progenitor to thereby convert the stem cell into the post-mitotic neuron or any progenitor into its differentiated cell, e.g., lung progenitor to differentiated lung cell.

In one aspect, provided herein are methods of converting progenitor cell into a differentiated cell (e.g., a lung progenitor into a lung cell) comprising introducing a nucleic acid expressing a HOXB7 protein into the progenitor to thereby convert the stem cell into the differentiated cell.

In certain methods, nucleic acid incorporates into the chromosomal DNA of the cell. For example, the DNA may be introduced by transfection or transduction and other methods known to the skilled artisan.

In one aspect, provided herein are uses of HOXB7, or homologues, derivatives or fragments thereof, for the manufacture of a medicament to treat HOXB7 related disorders.

Provided herein, according to one aspect, are pharmaceutical compositions comprising a pharmaceutically effective amount of a HOXB7 modulator effective to treat, prevent, ameliorate, reduce or alleviate a HOXB7 or symptoms thereof and a pharmaceutically acceptable excipient.

In one embodiment, the HOXB7 modulator is selected from one or more of a small molecule, an anti-HOXB7 antibody, an antigen-binding fragment of an anti-HOXB7 antibody, a polypeptide, a peptidomimetic, a nucleic acid encoding a peptide, or an organic molecule.

In another embodiment, the HOXB7 is cancer, infertility, pulmonary disease, tissue engineering, nerve damage, gastrointestinal disease, pain (chronic, neuropathic, acute), trauma, migraine, neurological disorders (anxiety, stroke, psychoses, schizophrenia, depression, epilepsy), cardiovascular conditions (hypertension and cardiac arrhythmias), diabetes, cancer, drug addiction, analgesic side effect, analgesic tolerance, diabetes, infertility, neurodegenerative disorders (e.g., ALS, Parkinson's Alzheimers, spinal cord injury and axonal regeneration, spinal bifida (neural tube closures)) or a behavioral disorder.

The present invention provides for both prophylactic and therapeutic methods of treating a subject at risk of, or susceptible to, a HOXB7 related disease or disorder. Treatment is defined as the application or administration of a therapeutic agent to a patient, or application or administration of a therapeutic agent to an isolated tissue or cell line from a patient, who has a HOXB7 related disease or disorder, a symptom of a HOXB7 related disease or disorder or a predisposition toward a HOXB7 related disease or disorder, with the purpose to cure, heal, alleviate, relieve, alter, remedy, ameliorate, improve or affect the disease or disorder, the symptoms of the disease or disorder or the predisposition toward the disease or disorder.

The therapeutic methods of the invention involve the administration of the polypeptide and/or nucleic acid molecules of the invention as described herein.

In one aspect, the invention provides a method for preventing a HOXB7 related disease or disorder in a subject by administering to the subject a polypeptide or nucleic acid molecule of the invention as described herein.

The invention provides therapeutic methods and compositions for the prevention and treatment of a HOXB7 related disease or disorder. In particular, the invention provides methods and compositions for the prevention and treatment of the disease or disorder in subjects.

In one embodiment, the present invention contemplates a method of treatment, comprising: a) providing, i.e., administering: i) a mammalian patient particularly human who has, or is at risk of developing a HOXB7 disease or disorder, ii) one or more molecules of the invention as described herein.

The term “at risk for developing” is herein defined as individuals an increased probability of contracting an HOXB7 related disease or disorder due to exposure or other health factors.

The present invention is also not limited by the degree of benefit achieved by the administration of the molecule. For example, the present invention is not limited to circumstances where all symptoms are eliminated. In one embodiment, administering a molecule reduces the number or severity of symptoms of a HOXB7 related disease or disorder. In another embodiment, administering of a molecule may delay the onset of symptoms of a HOXB7 related disease or disorder.

Yet another aspect of this invention relates to a method of treating a subject (e.g., mammal, human, horse, dog, cat, mouse) having a disease or disease symptom (including, but not limited to angina, hypertension, congestive heart failure, myocardial ischemia, arrhythmia, diabetes, urinary incontinence, stroke, pain, traumatic brain injury, or a neuronal disorder). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g., opinion) or objective (e.g., measurable by a test or diagnostic method).

Typical subjects for treatment in accordance with the individuals include mammals, such as primates, preferably humans. Cells treated in accordance with the invention also preferably are mammalian, particularly primate, especially human. As discussed above, a subject or cells are suitably identified as in needed of treatment, and the identified cells or subject are then selected for treatment and administered one or more of fusion molecules of the invention.

The treatment methods and compositions of the invention also will be useful for treatment of mammals other than humans, including for veterinary applications such as to treat horses and livestock e.g., cattle, sheep, cows, goats, swine and the like, and pets such as dogs and cats.

In other embodiments, the inhibition HOXB7 proteins can be achieved by any available means, e.g., inhibition of: (1) the expression, mRNA stability, protein trafficking, modification (e.g., phosphorylation), or degradation of an HOXB7 protein, or (2) one or more of the normal functions of an HOXB7 protein.

In one embodiment, HOXB7 protein inhibition is achieved by reducing the level of HOXB7 proteins in a tissue expressing the protein. Thus, the method of the invention can target HOXB7 proteins in tissues wherein the protein is expressed as described infra. This can be achieved using, e.g., antisense or RNA interference (RNAi) techniques to reduce the level of the RNA available for translation.

Methods of Screening

The role of HOXB7 proteins in mediating a HOXB7 related disorders makes the HOXB7 protein an attractive target for agents that modulate these disorders to effectively treat, prevent, ameliorate, reduce or alleviate the disorders. Accordingly, the invention provides prescreening and screening methods aimed at identifying such agents. The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof or in animals.

In one embodiment, therefore, a prescreening method comprises contacting a test agent with an HOXB7 protein. Such prescreening is generally most conveniently accomplished with a simple in vitro binding assay. Means of assaying for specific binding of a test agent to a polypeptide are well known to those of skill in the art and are detailed in the Examples infra. In one binding assay, the polypeptide is immobilized and exposed to a test agent (which can be labeled), or alternatively, the test agent(s) are immobilized and exposed to the polypeptide (which can be labeled). The immobilized species is then washed to remove any unbound material and the bound material is detected. To prescreen large numbers of test agents, high throughput assays are generally preferred. Various screening formats are discussed in greater detail below.

Test agents, including, for example, those identified in a prescreening assay of the invention can also be screened to determine whether the test agent affects the levels of HOXB7 proteins or RNA. Agents that reduce these levels can potentially reduce one or more HOXB7 related disorders.

Accordingly, the invention provides a method of screening for an agent that modulates a HOXB7 in which a test agent is contacted with a cell that expresses a HOXB7 protein in the absence of test agent. Preferably, the method is carried out using an in vitro assay or in vivo. In such assays, the test agent can be contacted with a cell in culture or to a tissue. Alternatively, the test agent can be contacted with a cell lysate or fraction thereof (e.g., a membrane fraction for detection of HOXB7 proteins or polypeptides thereof). The level of (i) HOXB7 proteins; or RNA is determined in the presence and absence (or presence of a lower amount) of test agent to identify any test agents that alter the level. If the level assayed is altered, the test agent is selected as a potential modulator of a HOXB7 related disorder. In a preferred embodiment, an agent that reduces or increases the level assayed is selected as a potential modulator of one or more HOXB7 related disorders.

Cells useful in this screening method include those from any of the species described above in connection with the method of reducing a drug-related effect or behavior. Cells that naturally express an HOXB7 protein are useful in this screening methods. Examples include MCF-7 cells, SKBR3 breast cancer cells, MDCK cells, epithelial cells, MCF10A cells, MCF-12A cells, MDA-MB-231 cells, PC12 cells, SH-SY5y cells, NG108-15 cells, IMR-32 cells, SK-N-SH cells, RINm5F cells, and NMB cells. Alternatively, cells that have been engineered to express a HOXB7 protein can be used in the method.

In one embodiment, the test agent is contacted with the cell in the presence of a drug. The drug is generally one that produces one or more undesirable effects or behaviors, such as, for example, sedative-hypnotic and analgesic drugs. In particular embodiments, the drug is ethanol, a cannabinioid, or an opioid.

As noted above, screening assays are generally carried out in vitro, for example, in cultured cells, in a biological sample (e.g., brain, dorsal root ganglion neurons, and sympathetic ganglion neurons), or fractions thereof. For ease of description, cell cultures, biological samples, and fractions are referred to as “samples” below. The sample is generally derived from an animal (e.g., any of the research animals mentioned above), preferably a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

HOXB7 proteins can be detected and quantified by any of a number of methods well known to those of skill in the art. Examples of analytic biochemical methods suitable for detecting HOXB7 protein, include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), receptor-linked immunosorbent assays (ELISAs), immunofluorescent assays, Western blotting, fluorescence resonance energy transfer (FRET) assays, yeast two-hybrid assays, whole or partial cell current recordings, and the like. Peptide modulators may be discovered or screened for example, by phage display. See 5,096,815; 5,198,346; 5,223,409; 5,260,203; 5,403,484; 5,534,621; and 5,571,698.

Methods for identifying lead compounds for a pharmacological agent useful in the treatment of a HOXB7 comprising contacting a HOXB7 protein with a test compound, and measuring cell survival, DNA repair, plasmid end joining, cologenic survival, expression of ER alpha, EGFR, HER2, Bcl-2, wound healing, invasion assays, and/or activation of Ras-MAP kinase pathways. The HOXB7 protein may also be a modified, e.g., a chimeric and/or a deletion mutant. The HOXB7 protein may be isolated or may be in a membrane or an artificial membrane. The contacting may be directly or indirectly.

Methods of the invention also include methods for screening a therapeutic agent to treat, prevent, ameliorate, reduce or alleviate a HOXB7 related disorders or symptoms thereof, comprising administering a test agent to a mouse having an over-expressed HOXB7 protein.

The proteinaceous molecules described above may be derived from any suitable source such as natural, recombinant or synthetic sources and includes fusion proteins or molecules which have been identified following, for example, natural product screening or high-throughput screening. The reference to non-proteinaceous molecules may be, for example, a reference to a nucleic acid molecule or it may be a molecule derived from natural sources, such as for example natural product screening, or may be a chemically synthesized molecule. The present invention contemplates analogues of the HOXB7 expression product or small molecules capable of acting as agonists or antagonists. Chemical agonists may not necessarily be derived from the HOXB7 expression product but may share certain conformational similarities. Alternatively, chemical agonists may be specifically designed to meet certain physiochemical properties. Antagonists may be any compound capable of blocking, inhibiting or otherwise preventing HOXB7 from carrying out its normal biological function, such as molecules which prevent its activation or else prevent the downstream functioning of activated HOXB7. Antagonists include monoclonal antibodies and antisense nucleic acids which prevent transcription or translation of HOXB7 genes or mRNA in mammalian cells. Modulation of expression may also be achieved utilizing antigens, RNA, ribosomes, DNAzymes, RNA aptamers, antibodies or molecules suitable for use in co-suppression. The proteinaceous and non-proteinaceous molecules referred to in points (i)-(v), above, are herein collectively referred to as “modulatory agents”. In another embodiment, the HOXB7 modulator is one or more of a small molecule, an anti-HOXB7 antibody, an antigen-binding fragment of an anti-HOXB7 antibody, a polypeptide, a peptidomimetic, a nucleic acid encoding a peptide, or an organic molecule.

Screening for the modulatory agents can be achieved by any one of several suitable methods including, but in no way limited to, contacting a cell comprising the HOXB7 gene or functional equivalent or derivative thereof with an agent and screening for the modulation of HOXB7 protein production or functional activity, modulation of the expression of a nucleic acid molecule encoding HOXB7 or modulation of the activity or expression of a downstream HOXB7 cellular target, cell survival, DNA repair, plasmid end joining, cologenic survival, expression of ER alpha, EGFR, HER2, Bcl-2, wound healing, invasion assays, and/or activation of Ras-MAP kinase pathways. Detecting such modulation can be achieved utilizing techniques such as Western blotting, electrophoretic mobility shift assays and/or the readout of reporters of HOXB7 activity such as luciferases, CAT and the like or observation of morphological changes.

The HOXB7 gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate HOXB7 activity, at either the nucleic acid or expression product levels, or the gene may require activation—thereby providing a model useful for, inter alia, screening for agents which up regulate HOXB7 expression. Further, to the extent that a HOXB7 nucleic acid molecule is transfected into a cell, that molecule may comprise the entire HOXB7 gene or it may merely comprise a portion of the gene such as the portion which regulates expression of the HOXB7 product. For example, the HOXB7 promoter region may be transfected into the cell which is the subject of testing. In this regard, where only the promoter is utilized, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream HOXB7 regulatory target, rather than HOXB7 itself. Yet another example includes HOXB7 binding sites ligated to a minimal reporter. For example, modulation of HOXB7 activity can be detected by screening for the modulation of the functional activity in a cell. This is an example of an indirect system where modulation of HOXB7 expression, per se, is not the subject of detection. Rather, modulation of the molecules which HOXB7 regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the HOXB7 nucleic acid molecule or expression product itself or which modulate the expression of an upstream molecule, which upstream molecule subsequently modulates HOXB7 expression or expression product activity. Accordingly, these methods provide a mechanism for detecting agents which either directly or indirectly modulate HOXB7 expression and/or activity.

The agents which are utilized in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, The agent is associated with a molecule which permits its targeting to a localized region.

The proteinaceous or non-proteinaceous molecules may act either directly or indirectly to modulate the expression of HOXB7 or the activity of the HOXB7 expression product. The molecule acts directly if it associates with the HOXB7 nucleic acid molecule or expression product to modulate expression or activity, respectively. The molecule acts indirectly if it associates with a molecule other than the HOXB7 nucleic acid molecule or expression product which other molecule either directly or indirectly modulates the expression or activity of the HOXB7 nucleic acid molecule or expression product, respectively. Accordingly, the method of the present invention encompasses the regulation of HOXB7 nucleic acid molecule expression or expression product activity via the induction of a cascade of regulatory steps.

The term “expression” refers, for example, to the transcription and translation of a nucleic acid molecule. Reference to “expression product” is a reference to the product produced from the transcription and translation of a nucleic acid molecule.

“Derivatives” of the molecules herein described (for example HOXB7 or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, to increase or otherwise enhance the rate and volume of production by that particular cellular source. Parts or fragments include, for example, active regions of the molecule. Derivatives may be derived from insertion, deletion or substitution of amino acids Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, HOXB7 or derivative thereof may be fused to a molecule to facilitate its entry into a cell. Analogues of the molecules contemplated herein include, for example, modification to side chains, incorporating of unnatural amino acids and/or their derivatives during peptide, polypeptide or protein synthesis and the use of crosslinkers and other methods including conformational constraints on the proteinaceous molecules or their analogues.

Derivatives of nucleic acid sequences which may be utilized in accordance with the method described herein may similarly be derived from single or multiple nucleotide substitutions, deletions and/or additions including fusion with other nucleic acid molecules. The derivatives of the nucleic acid molecules utilized as described herein include, for example, oligonucleotides, PCR primers, antisense molecules, molecules suitable for use in co-suppression and fusion of nucleic acid molecules. Derivatives of nucleic acid sequences also include degenerate variants.

A “variant” of HOXB7 should be understood to include, for example, molecules that exhibit at least some of the functional activity of the form of HOXB7 of which it is a variant. A variation may take any form and may be naturally or non-naturally occurring. A mutant molecule is one which exhibits, for example, modified functional activity.

A “homologue” is includes, for example, that the molecule is derived from a species other than that which is being treated in accordance with the method of the present invention. This may occur, for example, where it is determined that a species other than that which is being treated produces a form of HOXB7 which exhibits similar and suitable HOXB7 expression to that of the HOXB7 which is naturally produced by the subject undergoing treatment.

Chemical and functional equivalents include, for example, molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

In one aspect, provided herein are methods for screening a therapeutic agent to treat, prevent, ameliorate, reduce or alleviate a HOXB7 related disorder (e.g., DNA repair disorder, cancer or estrogen-response modulator resistance) or symptoms thereof, comprising administering a test agent to a mouse having an over-expressed HOXB7 protein, and measuring modulation of HOXB7 expression. In one aspect, provided herein are methods for identifying lead compounds for a pharmacological agent useful in the treatment of a HOXB7 comprising contacting a cell expressing a HOXB7 protein with a test compound, and measuring HOXB7 expression, modulation, cell survival, DNA repair, plasmid end joining, cologenic survival, expression of ER alpha, EGFR, HER2, Bcl-2, wound healing, invasion assays, and/or activation of Ras-MAP kinase pathways.

In one aspect, provided herein are methods for identifying lead compounds for a pharmacological agent useful in the treatment of a HOXB7 comprising contacting a cell that does not express a functional amount of a HOXB7 protein with a test compound, and measuring one or more of HOXB7 expression or cell survival, DNA repair, plasmid end joining, cologenic survival, expression of ER alpha, EGFR, HER2, Bcl-2, wound healing, invasion assays, and/or activation of Ras-MAP kinase pathways.

In another embodiment, the test compounds is one or more of a peptide, a small molecule, an antibody or fragment thereof, and nucleic acid or a library thereof.

Also useful in the screening techniques described herein are combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing HOXB7 analogues which exhibit properties such as more potent pharmacological effects.

With respect to high throughput library screening methods, oligomeric or small-molecule library compounds capable of interacting specifically with a selected biological agent, such as a biomolecule, a macromolecule complex, or cell, are screened utilizing a combinational library device which is easily chosen by the person of skill in the art from the range of well-known methods, such as those described above. In such a method, each member of the library is screened for its ability to interact specifically with the selected agent. In practicing the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

Analogues of HOXB7 or of HOXB7 agonistic or antagonistic agents contemplated herein include, for example, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

High Throughput Screening Assays

High throughput screening (HTS) typically uses automated assays to search through large numbers of compounds for a desired activity. Typically HTS assays are used to find new drugs by screening for chemicals that act on a particular receptor or molecule. For example, if a chemical inactivates an receptor it might prove to be effective in preventing a process in a cell which causes a disease. High throughput methods enable researchers to try out thousands of different chemicals against each target very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day. Examples include the yeast two-hybrid system and phage display. For examples of phage display see, U.S. Pat. Nos. 5,096,815; 5,198,346; 5,223,409; 5,260,203; 5,403,484; 5,534,621; and 5,571,698.

Screening assays may include controls for purposes of calibration and confirmation of proper manipulation of the components of the assay. Blank wells that contain all of the reactants but no member of the chemical library are usually included. As another example, a known modulator (or activator) of an receptor for which modulators are sought, can be incubated with one sample of the assay, and the resulting decrease (or increase) in the receptor activity determined according to the methods herein. It will be appreciated that modulators can also be combined with the receptor activators or modulators to find modulators which inhibit the receptor activation or repression that is otherwise caused by the presence of the known the receptor modulator. Similarly, when ligands to a sphingolipid target are sought, known ligands of the target can be present in control/calibration assay wells.

Measuring Binding Reactions During Screening Assays

Techniques for measuring the progression of binding reactions in multicontainer carriers are known in the art and include, but are not limited to, the following.

Spectrophotometric and spectrofluorometric assays are well known in the art. Examples of such assays include the use of colorimetric assays for the detection of peroxides, as disclosed in Example 1(b) and Gordon, A. J. and Ford, R. A., The Chemist's Companion: A Handbook Of Practical Data, Techniques, And References, John Wiley and Sons, N.Y., 1972, Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., Spectrophotometry and Spectrofluorometry: A Practical Approach, pp. 91-114, IRL Press Ltd. (1987); and Bell, Spectroscopy In Biochemistry, Vol. I, pp. 155-194, CRC Press (1981).

In spectrofluorometric methods, receptors are exposed to substrates that change their intrinsic fluorescence when processed by the target receptor. Typically, the substrate is nonfluorescent and converted to a fluorophore through one or more reactions. As a non-limiting example, SMase activity can be detected using the Amplex® Red reagent (Molecular Probes, Eugene, Oreg.). In order to measure sphingomyelinase activity using Amplex Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (e.g., a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

Fluorescence resonance energy transfer (FRET) is another useful assay for detecting interaction and has been described previously. See, e.g., Heim et al., Curr. Biol. 6:178-182, 1-996; Mitra et al., Gene 173:13-17 1996; and Selvin et al., Meth. Enzymol. 246:300-345, 1995. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., J. Lipid Res. 38:2365-2373 (1997); Kahl et al., Anal. Biochem. 243:282-283 (1996); Undenfriend et al., Anal. Biochem. 161:494-500 (1987)). See also U.S. Pat. Nos. 4,626,513 and 4,568,649, and European Patent No. 0,154,734. One commercially available system uses FLASHPLATE scintillant-coated plates (NEN Life Science Products, Boston, Mass.).

In certain embodiments, HOXB7 polypeptide(s) are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., 3H, 125I, 35S, 14C, or 32P), receptors (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In preferred embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Changes in HOXB7 protein subunit expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In one embodiment, amplification-based assays can be used to detect, and optionally quantify, a polynucleotide encoding a HOXB7 protein of interest. In such amplification-based assays, the mRNA in the sample act as template(s) in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of the HOXB7 mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of HOXB7 mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the HOXB7 mRNA of interest, and standard methods (such as, e.g., densitometry where the nucleic acid probe is radioactively labeled) can be used to quantify the level of the HOXB7 polynucleotide.). A variety of additional nucleic acid hybridization formats are known to those skilled in the art. Standard formats include sandwich assays and competition or displacement assays. Sandwich assays are commercially useful hybridization assays for detecting or isolating polynucleotides.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211). See also, for example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

Hybridization assays according to the invention can also be carried out using a MicroElectroMechanical System (MEMS), such as the Protiveris' multicantilever array.

HOXB7 RNA is detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Test Agents Identified by Screening

When a test agent is found to modulate one or more HOXB7 proteins, or RNA. A preferred screening method of the invention further includes combining the test agent with a carrier, preferably pharmaceutically acceptable carrier, such as are described above. Generally, the concentration of test agent is sufficient to alter the level of HOXB7 proteins or RNA, or HOXB7 expression. This concentration will vary, depending on the particular test agent and specific application for which the composition is intended. As one skilled in the art appreciates, the considerations affecting the formulation of a test agent with a carrier are generally the same as described above with respect to methods of reducing a drug-related effect or behavior.

In a preferred embodiment, the test agent is administered to an animal to measure the ability of the selected test agent to modulate a drug-related effect or behavior in a subject, as described in greater detail below.

Preferred compositions for use in the therapeutic methods of the invention inhibit the HOXB7 protein function by about 5% based on, for example, compound state analysis techniques or modulatory profiles described infra, more preferably about 7.5% or 10% inhibition or initiation of HOXB7 expression of the cell, and still more preferable, at least about 20%, 30%, 40%, 50%, 60%, 70%, 80%, 90% or 100% initiation or inhibition of HOXB7 expression.

Compositions

Soluble polypeptides derived from HOXB7 protein that retain the ability to initiate differentiation are useful. In addition, modification of such residues may permit the skilled artisan to tailor the binding specificities and/or affinity of polypeptides.

The HOXB7 proteins are of particular interest because they are of interest in the treatment, prevention, amelioration, reduction or alleviation of diseases.

The polypeptides may be prepared in various ways including, for example, molecular biological techniques, including proteolytic digestion of cells or cellular membrane preparations comprising the receptor (Bartfeld et al., Active acetylcholine receptor fragment obtained by tryptic digestion of acetylcholine receptor from Torpedo californica, Biochem Biophys Res Commun. 89:512-9, 1979; Borhani et al., Crystallization and X-ray diffraction studies of a soluble form of the human transferrin receptor, J. Mol. Biol. 218:685-9, 1991), recombinant DNA technologies (Marlovits et al., Recombinant soluble low-density lipoprotein receptor fragment inhibits common cold infection, J Mol Recognit. 11:49-51, 1998; Huang et al., Expression of a human thyrotrophin receptor fragment in Escherichia coli and its interaction with the hormone and autoantibodies from subjects with Graves' disease, J Mol Endocrinol. 8:137-44, 1992), or by in vitro synthesis of oligopeptides.

Peptidomimetics

In general, a polypeptide mimetic (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides and peptoids. Examples of some peptidomimetics by the broader definition (e.g., where part of a polypeptide is replaced by a structure lacking peptide bonds) are described below. Whether completely or partially non-peptide in character, peptidomimetics according to this invention may provide a spatial arrangement of reactive chemical moieties that closely resembles the three-dimensional arrangement of active groups in a polypeptide. As a result of this similar active-site geometry, the peptidomimetic may exhibit biological effects that are similar to the biological activity of a polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that may be obviated with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure, shape or reactivity. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference].

Specific examples of peptidomimetics are set forth below. These examples are illustrative and not limiting in terms of the other or additional modifications.

Peptides with a Reduced Isostere Pseudopeptide Bond

Proteases act on peptide bonds. Substitution of peptide bonds by pseudopeptide bonds may confer resistance to proteolysis or otherwise make a compound less labile. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., (1993), Int. J. Polypeptide Protein Res. 41:181-184, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus

Peptides with a Retro-Inverso Pseudopeptide Bond

To confer resistance to proteolysis, peptide bonds may also be substituted by retro-inverso pseudopeptide bonds (Dalpozzo, et al. (1993), Int. J. Polypeptide Protein Res. 41:561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid Derivatives

Peptoid derivatives of polypeptides represent another form of modified polypeptides that retain the structural determinants for biological activity, yet eliminate the peptide bonds, thereby conferring resistance to proteolysis (Simon, et al., 1992, Proc. Natl. Acad. Sci. USA, 89:9367-9371 and incorporated herein by reference). Peptoids are oligomers of N-substituted glycines. A number of N-alkyl groups have been described, each corresponding to the side chain of a natural amino acid.

One of skill in the art can identify other peptides and understands that homologues and orthologues of these molecules are useful in the compositions and methods of the instant invention. Moreover, variants of the peptides, are useful in the methods and compositions of the invention.

One of skill in the art will understand that molecules that share one or more functional activities with the molecules identified above, but have differences in amino acid or nucleic acid sequence would be useful in the compositions and methods of the invention. For example, in a preferred embodiment, a polypeptide or biologically active fragment thereof has at least about 60%, 70%, 80%, 90%, 95%, 96%, 97%, 98%, 99%, or 100% identity with the polypeptide, or a fragment or variant thereof.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989, CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences that one of skill in the art could use to make the molecules of the invention. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to 13245 nucleic acid molecules of the invention. BLAST protein searches can be performed with the XBLAST program, score=50, wordlength=3 to obtain amino acid sequences homologous to 13245 protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used.

Vectors

Another aspect of the invention pertains to vectors, preferably expression vectors, containing a nucleic acid molecule encoding the fusion molecules, or components thereof, of the invention as described above. As used herein, the term “vector” refers to a nucleic acid molecule capable of transporting another nucleic acid molecule to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule of the invention in a form suitable for expression of the nucleic acid molecule in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., fusion molecules comprising a chemokine receptor ligand and a toxin moiety).

The recombinant expression vectors of the invention can be designed for expression of the polypeptides of the invention in prokaryotic or eukaryotic cells. For example, the polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Another aspect of the invention pertains to host cells into which a nucleic acid molecule encoding a fusion polypeptide of the invention is introduced within a recombinant expression vector or a nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a fusion polypeptide of the invention can be expressed in bacterial cells such as E. coli, insect cells, yeast or mammalian cells (such as Chinese hamster ovary cells (CHO) or COS cells). Other suitable host cells are known to those skilled in the art.

Methods of Making the Molecules of the Invention

As described above, molecules of the invention may be made recombinantly using the nucleic acid molecules, vectors, host cells and recombinant organisms described above.

Alternatively, the peptide can be made synthetically, or isolated from a natural source and linked to the carbohydrate recognition domain using methods and techniques well known to one of skill in the art.

Further, to increase the stability or half life of the fusion molecules of the invention, the polypeptides may be made, e.g., synthetically or recombinantly, to include one or more peptide analogs or mimetics. Exemplary peptides can be synthesized to include D-isomers of the naturally occurring amino acid residues or amino acid analogs to increase the half life of the molecule when administered to a subject.

Pharmaceutical Compositions

The nucleic acid and polypeptide fusion molecules (also referred to herein as “active compounds”) of the invention can be incorporated into pharmaceutical compositions. Such compositions typically include the nucleic acid molecule or protein, and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions of the instant invention may also include one or more other active compounds. Alternatively, the pharmaceutical compositions of the invention may be administered with one or more other active compounds. Other active compounds that can be administered with the pharmaceutical compounds of the invention, or formulated into the pharmaceutical compositions of the invention, include, for example, anti-inflammatory compounds.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Preferred pharmaceutical compositions of the invention are those that allow for local delivery of the active ingredient, e.g., delivery directly to the location of a tumor. Although systemic administration is useful in certain embodiments, local administration is preferred in most embodiments.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, kit or dispenser together with instructions, e.g., written instructions, for administration, particularly such instructions for use of the active agent to treat against a disorder or disease as disclosed herein, including a HOXB7 related disorder. The container, pack, kit or dispenser may also contain, for example, a nucleic acid sequence encoding a peptide, or a peptide expressing cell.

For research and therapeutic applications, an HOXB7 protein modulator is generally formulated to deliver modulator to a target site in an amount sufficient to inhibit HOXB7 proteins at that site.

Modulator compositions or peptides of the invention optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Certain embodiments include sustained-release pharmaceutical compositions. An exemplary sustained-release composition has a semipermeable matrix of a solid hydrophobic polymer to which the modulator is attached or in which the modulator is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are in the form of shaped articles, such as films, or microcapsules.

Where the modulator is a polypeptide, exemplary sustained release compositions include the polypeptide attached, typically via epsilon-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” variant retains the desired function(s).

In another embodiment, a sustained-release composition includes a liposomally entrapped modulator. Liposomes are small vesicles composed of various types of lipids, phospholipids, and/or surfactants. These components are typically arranged in a bilayer formation, similar to the lipid arrangement of biological membranes. Liposomes containing HOXB7 protein modulators are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions can also include an modulator adsorbed onto a membrane, such as a silastic membrane, which can be implanted, as described in International Publication No. WO 91/04014.

Pharmaceutical compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

In particular embodiments, the methods of the invention employ pharmaceutical compositions containing a polynucleotide encoding a polypeptide modulator of HOXB7 proteins. Such compositions optionally include other components, as for example, a storage solution, such as a suitable buffer, e.g., a physiological buffer. In a preferred embodiment, the composition is a pharmaceutical composition and the other component is a pharmaceutically acceptable carrier as described above.

Preferably, compositions containing polynucleotides useful in the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)pro-pyl]-n,n,n-trimethylammonium chloride (DOTMA), dioleoyl phosphotidylethanolamine (DOPE), and/or dioleoyl phosphatidylcholine (DOPC). Polynucleotides can also be entrapped in liposomes, as described above.

In another embodiment, polynucleotides are complexed to dendrimers, which can be used to introduce polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides useful in the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides.

Transgenic Animals

The transgenic non-human animal may be a primate, mouse, dog, cat, sheep, horse, rabbit or other non-human animal. Cells may be isolated and cultured from the transgenic non-human animals. The cells may be used in, for example, primary cultures or established cultures. In one aspect, provided herein are uses of a transgenic animal as described herein to test therapeutic agents.

In another embodiment, a decrease HOXB7 expression indicates that the test agent may be useful in treating a HOXB7 disorder or changes in GDPD enzymatic activity.

A transgenic non-human animal comprising an over-expressed NTB peptide or a fragment or variant thereof. The use of a transgenic animal according to claim 50, to test therapeutic agents. Embodiments of the invention include the use of the ES cell lines derived from the transgenic zygote, embryo, blastocyst or non-human animal to treat human and non-human animal diseases.

Transgenic non-human animals include those whose genome comprises over-expressed NT_{B} peptide or a fragment or variant thereof. The methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; and Pinkert, Calif., Ed., Transgenic Animal Technology: A Laboratory Handbook, Academic Press, 1994. In certain embodiments, transgenic mice will be produced as described in Thomas et al. (1999) Immunol., 163:978-84; Kanakaraj et al. (1998) J. Exp. Med., 187:2073-9; or Yeh et al. (1997) Immunity 7:715-725. Methods of producing the transgenic animals are well-known in the art. See for example, Hooper, M L, Embryonal Stem Cells: Introducing Planned Changes into the Animal Germline (Modeem Genetics, v. 1), Inf. Pub. Distrib., Inc., 1993; Bradley et al. (1984) Nature, 309, 255-258; Jaenisch (1988) Science, 240:1468-1474; Wilmut et al. (1997) Nature, 385: 810-813; DeBoer et al., WO 91/08216; Wang, et al. Molecular Reproduction and Development (2002) 63:437-443); Page, et al. Transgenic Res (1995) 4(6):353-360; Lebkowski, et al. Mol Cell Biol (1988) 8(10):3988-3996; “Molecular Cloning: A Laboratory Manual. Second Edition” by Sambrook, et al. Cold Spring Harbor Laboratory: 1989; “Transgenic Animal Technology: A Laboratory Handbook,” C. A. Pinkert, editor, Academic Press, 2002, 2nd edition, 618 pp.; “Mouse Genetics and Transgenics: A Practical Approach,” I. J. Jackson and C. M. Abbott, editors, Oxford University Press, 2000, 299 pp.; “Transgenesis Techniques: Principles and Protocols,” A. R. Clarke, editor, Humana Press, 2001, 351 pp.; Velander et al., Proc. Natl. Acad. Sci. USA 89:12003-12007, 1992; Hammer et al., Nature 315:680-683, 1985; Gordon et al., Science 214:1244-1246, 1981; and Hogan et al., Manipulating the Mouse Embryo: A Laboratory Manual (Cold Spring Harbor Laboratory, 2002), which are each incorporated herein by reference in their entirety.

Cells obtained from the transgenic non-human animals described herein may be obtained by taking a sample of a tissue of the animal. The cells may then be cultured. The cells preferably lack production of functional protein encoded by the nucleotide sequence or a fragments or variants thereof.

In one embodiment, the transgenic non-human animal is a male non-human animal. In other preferred embodiments the transgenic non-human animal is a female non-human animal. According to other embodiments, the transgenic non-human animal oocyte, blastocyst, embryo, or offspring may be used as a model for a human disease, as a model to study human disease or to screen molecules, compounds and compositions. In certain embodiments, the cells of the transgenic oocyte, zygote, blastocyst, or embryo are used to establish embryonic stem (ES) cell lines. Stem cells are defined as cells that have extensive proliferation potential, differentiate into several cell lineages, and repopulate tissues upon transplantation. (Thomson, J. et al. 1995; Thomson, J. A. et al. 1998; Shamblott, M. et al. 1998; Williams, R. L. et al. 1988; Orkin, S. 1998; Reubinoff, B. E., et al. 2000).

Described herein below in Example 4 is a MMTV-HOXB7 transgenic mouse model was established. The full-length mouse Hoxb7 gene was inserted downstream of MMTV promoter, which drives gene expression in mammary epithelial cells. Other HOXB7 animals are envisioned and one of skill in the art would know how to created such other transgenic animals.

Kits

The invention also provides kits useful in practicing the methods of the invention. In one embodiment, a kit of the invention includes a HOXB7 protein modulator, e.g., contained in a suitable container. Provided herein, according to one aspect, are kits comprising an HOXB7 modulator and a pharmaceutically acceptable carrier and b) instructions for use. In a variation of this embodiment, the HOXB7 protein modulator is formulated in a pharmaceutically acceptable carrier. The kit preferably includes instructions for administering the N-type modulator to a subject to reduce or prevent a drug-related effect or behavior.

Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

RNAi Compositions for Targeting HOXB7 mRNA

RNAi molecules may interfere with any portion of the mRNA of HOXB7. As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which directs the degrading mechanism to other similar RNA sequences. Alternatively, RNAi can be initiated by the hand of man, for example, to silence the expression of target genes. RNAi molecules useful for RNAi are sometime referred to herein as small interfering RNAs (siRNA).

By “reduce or inhibit” is meant the ability to cause an overall decrease preferably of 20% or greater, more preferably of 50% or greater, and most preferably of 75% or greater, in the level of protein or nucleic acid, detected by the aforementioned assays (see “expression”), as compared to samples not treated with antisense nucleotide oligomers or dsRNA used for RNA interference.

An siRNA having a “sequence sufficiently complementary to a target mRNA sequence to direct target-specific RNA interference (RNAi)” means that the ss-siRNA has a sequence sufficient to trigger the destruction of the target mRNA by the RNAi machinery or process. Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing a siRNA of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

By “small interfering RNAs (siRNAs)” (also referred to in the art as “short interfering RNAs”) is meant an isolated RNA molecule comprising between about 10-50 nucleotides (or nucleotide analogs), which is capable of directing or mediating RNA interference. The siRNA is preferably greater than 10 nucleotides in length, more preferably greater than 15 nucleotides in length, and most preferably greater than 19 nucleotides in length that is used to identify the target gene or mRNA to be degraded. A range of 19-25 nucleotides is the most preferred size for siRNAs. siRNAs can also include short hairpin RNAs in which both strands of an siRNA duplex are included within a single RNA molecule. siRNA includes any form of dsRNA (specifically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the 21 to 23 nt RNA or internally (at one or more nucleotides of the RNA). In a preferred embodiment, the RNA molecules contain a 3′ hydroxyl group. Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA that it has the ability to mediate RNA interference (RNAi). RNAi agents of the present invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymidine transcription termination site. Upon expression, shRNAs are thought to fold into a stem-loop structure with 3′ UU-overhangs; subsequently, the ends of these shRNAs are processed, converting the shRNAs into siRNA-like molecules of about 21-23 nucleotides. (Brummelkamp et al., Science 296:550-553 (2002); Lee et al, (2002). supra; Miyagishi and Taira, Nature Biotechnol. 20:497-500 (2002); Paddison et al. (2002), supra; Paul (2002), supra; Sui (2002) supra; Yu et al. (2002), supra).

siRNAs also include “single-stranded small interfering RNA molecules. “Single-stranded small interfering RNA molecules” (“ss-siRNA molecules” or “ss-siRNA”). ss-siRNA is an active single stranded siRNA molecule that silences the corresponding gene target in a sequence specific manner. Preferably, the ss-siRNA molecule has a length from about 10-50 or more nucleotides. More preferably, the ss-siRNA molecule has a length from about 19-23 nucleotides. In addition to compositions comprising ss-siRNA molecules other embodiments of the invention include methods of making said ss-siRNA molecules and methods (e.g., research and/or therapeutic methods) for using said ss-siRNA molecules.

As used herein, the term “specifically hybridizes” or “specifically detects” refers to the ability of a nucleic acid molecule to hybridize to at least approximately 6 consecutive nucleotides of a sample nucleic acid.

A “target gene” is a gene whose expression is to be selectively inhibited or “silenced,” for example, HOXB7. This silencing is achieved by cleaving the mRNA of the target gene by an siRNA that is created from an engineered RNA precursor by a cell's RNAi system. One portion or segment of a duplex stem of the RNA precursor is an anti-sense strand that is complementary, e.g., fully complementary, to a section of about 18 to about 40 or more nucleotides of the mRNA of the target gene.

This invention is generally related to treatment and management of angiogenesis by using the HOXB7 members' genes and their products by inhibiting their expression. One embodiment of this invention is directed to a method comprising contacting the cell with a compound that inhibits the synthesis or expression of HOXB7 genes in an amount sufficient to cause such inhibition. Without being limited by theory, the inhibition is achieved through selectively targeting HOXB7 members' DNA or mRNA, i.e., by impeding any steps in the replication, transcription, splicing or translation of the genes. The sequence of are disclosed in GenBank Accession Nos. disclosed above, which are hereby incorporated by reference in their entirety.

RNAi is a remarkably efficient process whereby double-stranded RNA (dsRNA) induces the sequence-specific degradation of homologous mRNA in animals and plant cells (Hutvagner and Zamore (2002), Curr. Opin. Genet. Dev., 12, 225-232; Sharp (2001), Genes Dev., 15, 485-490). In mammalian cells, RNAi can be triggered by 21-nucleotide (nt) duplexes of small interfering RNA (siRNA) (Chiu et al. (2002), Mol. Cell., 10, 549-561; Elbashir et al. (2001), Nature, 411, 494-498), or by micro-RNAs (miRNA), functional small-hairpin RNA (shRNA), or other dsRNAs which are expressed in-vivo using DNA templates with RNA polymerase III promoters (Zeng et al. (2002), Mol. Cell, 9, 1327-1333; Paddison et al. (2002), Genes Dev., 16, 948-958; Lee et al. (2002), Nature Biotechnol., 20, 500-505; Paul et al. (2002), Nature Biotechnol., 20, 505-508; Tuschl, T. (2002), Nature Biotechnol., 20, 440-448; Yu et al. (2002), Proc. Natl. Acad. Sci. USA, 99(9), 6047-6052; McManus et al. (2002), RNA, 8, 842-850; Sui et al. (2002), Proc. Natl. Acad. Sci. USA, 99(6), 5515-5520.)

The present invention features “small interfering RNA molecules” (“siRNA molecules” or “siRNA”), methods of making said siRNA molecules and methods (e.g., research and/or therapeutic methods) for using said siRNA molecules. A siRNA molecule of the invention is a duplex consisting of a sense strand and complementary antisense strand, the antisense strand having sufficient complementary to a target mRNA to mediate RNAi. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region, such as a target region that differs by at least one base pair between the wild type and mutant allele, e.g., a target region comprising the gain-of-function mutation, and the other strand is identical or substantially identical to the first strand. small interfering RNA molecules

In one embodiment, the expression of HOXB7 is inhibited by the use of an RNA interference technique referred to as RNAi. RNAi allows for the selective knockdown of the expression of a target gene in a highly effective and specific manner. This technique involves introducing into a cell double-stranded RNA (dsRNA), having a sequence corresponding to the exon portion of the target gene. The dsRNA causes a rapid destruction of the target gene's mRNA. See, e.g., Hammond et al., Nature Rev Gen 2: 110-119 (2001); Sharp, Genes Dev 15: 485-490 (2001), both of which are incorporated herein by reference in their entireties.

Methods and procedures for successful use of RNAi technology are well-known in the art, and have been described in, for example, Waterhouse et al., Proc. Natl. Acad. Sci. USA 95(23): 13959-13964 (1998). The siRNAs of this invention encompass any siRNAs that can modulate the selective degradation of HOXB7 mRNAs.

The siRNAs of the invention include “double-stranded small interfering RNA molecules” (“ds-siRNA” and “single-stranded small interfering RNA molecules” (“ss-siRNA”), methods of making the siRNA molecules and methods (e.g., research and/or therapeutic methods) for using the siRNA molecules.

Similarly to the ds-siRNA molecules, the ss-siRNA molecule has a length from about 10-50 or more nucleotides. More preferably, the ss-siRNA molecule has a length from about 15-45 nucleotides. Even more preferably, the ss-siRNA molecule has a length from about 19-40 nucleotides. The ss-siRNA molecules of the invention further have a sequence that is “sufficiently complementary” to a target mRNA sequence to direct target-specific RNA interference (RNAi), as defined herein, i.e., the ss-siRNA has a sequence sufficient to trigger the destruction of the target mRNA by the RNAi machinery or process. The ss-siRNA molecule can be designed such that every residue is complementary to a residue in the target molecule. Alternatively, substitutions can be made within the molecule to increase stability and/or enhance processing activity of a said molecule. Substitutions can be made within the strand or can be made to residues at the ends of the strand. The 5′-terminus is, most preferably, phosphorylated (i.e., comprises a phosphate, diphosphate, or triphosphate group). The 3′ end of a siRNA may be a hydroxyl group in order to facilitate RNAi, as there is no requirement for a 3′ hydroxyl group when the active agent is a ss-siRNA molecule. Featured are ss-siRNA molecules wherein the 3′ end (i.e., C3 of the 3′ sugar) lacks a hydroxyl group (i.e., ss-siRNA molecules lacking a 3′ hydroxyl or C3 hydroxyl on the 3′ sugar (e.g., ribose or deoxyribose).

The siRNAs of this invention include modifications to their sugar-phosphate backbone or nucleosides. These modifications can be tailored to promote selective genetic inhibition, while avoiding a general panic response reported to be generated by siRNA in some cells. Moreover, modifications can be introduced in the bases to protect siRNAs from the action of one or more endogenous enzymes.

The siRNAs of this invention can be enzymatically produced or totally or partially synthesized. Moreover, the siRNAs of this invention can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although 100 percent sequence identity between the siRNA and the target region is preferred, it is not required to practice this invention. siRNA molecules that contain some degree of modification in the sequence can also be adequately used for the purpose of this invention. Such modifications include, but are not limited to, mutations, deletions or insertions, whether spontaneously occurring or intentionally introduced. Specific examples of siRNAs that can be used to inhibit the expression of HOXB7.

The target RNA cleavage reaction guided by siRNAs is highly sequence specific. In general, siRNA containing a nucleotide sequences identical to a portion of the target gene are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition.

Moreover, not all positions of a siRNA contribute equally to target recognition. Mismatches in the center of the siRNA are most critical and essentially abolish target RNA cleavage. In contrast, the 3′ nucleotides of the siRNA do not contribute significantly to specificity of the target recognition. In particular, residue 3′ of the siRNA sequence which is complementary to the target RNA (e.g., the guide sequence) are not critical for target RNA cleavage.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17): 3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 90% sequence identity, e.g., 91%, 92%, 93%, 94%, 95%, 96%, 97%, 98%, 99% or even 100% sequence identity, between the siRNA and the portion of the target gene is preferred. Alternatively, the ss-siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50 degrees C. or 70 degrees C. hybridization for 12-16 hours; followed by washing). Additional preferred hybridization conditions include hybridization at 70 degrees C. in 1×SSC or 50 degrees C. in 1×SSC, 50% formamide followed by washing at 70 degrees C. in 0.3×SSC or hybridization at 70 degrees C. in 4×SSC or 50 degrees C. in 4×SSC, 50% formamide followed by washing at 67 degrees C. in 1×SSC. The hybridization temperature for hybrids anticipated to be less than 50 base pairs in length should be 5-10 degrees C. less than the melting temperature (Tm) of the hybrid, where Tm is determined according to the following equations. For hybrids less than 18 base pairs in length, Tm(degrees C.)=2(# of A+T bases)+4(# of G+C bases). For hybrids between 18 and 49 base pairs in length, Tm(degrees C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

In a preferred aspect, the RNA molecules of the present invention are modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

In an embodiment of the present invention the RNA molecule may contain at least one modified nucleotide analogue. The nucleotide analogues may be located at positions where the target-specific activity, e.g., the RNAi mediating activity is not substantially affected, e.g., in a region at the 5′-end and/or the 3′-end of the RNA molecule. Particularly, the ends may be stabilized by incorporating modified nucleotide analogues.

Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications might be combined.

The nucleic acid compositions of the invention include both siRNA and siRNA derivatives as described herein. For example, cross-linking can be employed to alter the pharmacokinetics of the composition, for example, to increase half-life in the body. Thus, the invention includes siRNA derivatives that include siRNA having two complementary strands of nucleic acid, such that the two strands are crosslinked. The invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Examples of suitable HOXB7 RNAi molecules are described below in the Examples.

All documents mentioned herein are incorporated by reference herein in their entirety.

### EXAMPLES

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### Example 1

Epithelial-mesenchymal transition (EMT), initially recognized as an essential step for embryogenesis in the early 1980s (9), is now considered a major mechanism for the conversion of early-stage tumors to invasive malignancies (4, 10-12). During passage through EMT, epithelial cells lose epithelial adherens and tight junction proteins, consequently lose polarity and cell-cell contacts, and undergo a dramatic remodeling of the cytoskeleton to facilitate cell motility and invasion (13). Transcriptional factors like Snail (10) and Twist (14, 15) were unveiled as key regulators in induction of EMT in breast cancer and other cancers and act by suppressing the expression of epithelial specific adhesion molecule, E-cadherin. E-cadherin expression is irreversibly lost in invasive lobular breast cancer (16). Besides these transcriptional factors, growth factors like hepatocyte growth factor (HGF; ref. 17), transforming growth factor (TGF)-β (18), and epidermal growth factor (EGF; ref. 19), as well as matrix metalloproteinase (MMP)-3 (20), also induce EMT in various cell lines. These studies also suggested that Snail was located at the hub of these growth factor signaling pathways leading to EMT because the activated receptor tyrosine kinases could up-regulate the expression of Snail by activating the Ras-mitogen-activated protein kinase (MAPK) pathway (21).

Herein, it is shown that HOXB7 is one of the promising candidate genes, which was overexpressed at increasingly higher levels from normal epithelial cells to primary metastatic breast tumors to bone metastatic lesions by microarray analysis of purified epithelial cells. HOXB7 was reported to be involved in tissue remodeling of the normal mammary gland (22) and was associated with the development of breast cancer (23, 24). cDNA-based comparative genomic hybridization revealed that HOXB7 was located in a novel amplicon at 17q21.3, and this amplification correlated with poor prognosis in a panel of 186 breast cancer cases (25). Overexpression of HOXB7 in SKBR3 breast cancer cells was found to directly or indirectly regulate the expression of many angiogenic and growth factors, including basic fibroblast growth factor (bFGF), vascular endothelial growth factor (VEGF), interleukin 8, Ang1, Ang2, and MMP9, and resulted in the formation of well-vascularized tumors when grown as xenografts in nude mice (23, 24).

Herein, it is shown that HOXB7 is overexpressed in primary breast carcinomas and distant metastasis to various organs. In cell culture models, we show the ability of HOXB7 to confer the biological and molecular characteristics of EMT to epithelial cells.

Epithelial-mesenchymal transition (EMT) is increasingly recognized as a mechanism whereby cells in primary noninvasive tumors acquire properties essential for migration and invasion. Microarray analyses of microdissected epithelial cells from bone metastasis revealed a HOXB7 overexpression that was 3-fold higher than in primary breast carcinomas and 18-fold higher compared with normal breast. This led us to investigate the role of HOXB7 in neoplastic transformation of breast cells. Expression of HOXB7 in both MCF10A and Madin-Darby canine kidney (MDCK) epithelial cells resulted in the acquisition of both phenotypic and molecular attributes typical of EMT. Loss of epithelial proteins, claudin 1 and claudin 7, mislocalization of claudin 4 and E-cadherin, and the expression of mesenchymal proteins, vimentin and α-smooth muscle actin, were observed. MDCK cells expressing HOXB7 exhibited properties of migration and invasion. Unlike MDCK vector-transfected cells, MDCK-HOXB7 cells formed highly vascularized tumors in mice. MDCK-HOXB7 cells overexpressed basic fibroblast growth factor (bFGF), had more active forms of both Ras and RhoA proteins, and displayed higher levels of phosphorylation of p44 and p42 mitogen-activated protein kinase (MAPK; extracellular signal-regulated kinases 1 and 2). Effects initiated by HOXB7 were reversed by specific inhibitors of FGF receptor and the Ras-MAPK pathways. These data provide support for a function for HOXB7 in promoting tumor invasion through activation of Ras/Rho pathway by up-regulating bFGF, a known transcriptional target of HOXB7. Reversal of these effects by HOXB7-specific siRNA further suggested that these effects were mediated by HOXB7. Thus, HOXB7 overexpression caused EMT in epithelial cells, accompanied by acquisition of aggressive properties of tumorigenicity, migration, and invasion. (Cancer Res 2006; 66(19): 9527-34).

Purification of epithelial cells, RNA amplification, and labeling for microarray. Epithelial cells were isolated from freshly resected mammoplasty tissue (normal breast tissue; n=2) by immunopurification. Malignant epithelial cells from frozen breast tissue specimens, primary invasive ductal carcinomas with lymph node metastasis (n=2), and from bone metastases (n=3) were purified by laser capture microdissection. Laser capture microdissection was carried out using a PixCell II LCM system (Arcturus Engineering, Mountain View, Calif.) as per instructions of the manufacturer. Total RNA from purified epithelial cells was extracted with RNeasy Mini kit (Qiagen, Inc., Valencia, Calif.) including a DNase treatment step. RNA (˜10 ng total RNA) from each sample was amplified with the RiboAmp RNA Amplification kit (Arcturus Engineering). The amplified RNA was labeled with the ENZO BioArray HighYield RNA transcript labeling kit (Affymetrix, Santa Clara, Calif.). To obtain appropriate concentrations for hybridization, three bone metastasis samples were pooled. Biotin-labeled RNA samples (12 μg RNA of normal, invasive ductal carcinoma, and pooled bone metastases) were then fragmented and hybridized to the GeneChip Human Genome U133A 2.0 Array (Affymetrix). Reverse transcription-PCR, quantitative PCR, and statistical analysis. Immortalized cell lines derived from normal human mammary epithelial cells, MCF10A, normal Madin-Darby canine kidney (MDCK) breast cancer cell lines (American Type Culture Collection, Manassas, Va.), normal organoid, and tumor RNA were extracted by Trizol method, and all cDNAs were prepared with 1 μg of RNA in SuperScript II (Invitrogen, Carlsbad, Calif.) reactions according to the instructions of the manufacturer. Reverse transcription-PCR (RT-PCR) amplifications of HOXB7, bFGF, and internal control gene 36B4 were done with the following primer pairs: HOXB7, forward 5′-AGAGTAACTTCCGGATCTA-3′ (SEQ ID NO: 3) and reverse 5′-TCGGCTTCAGCCCTGTCTT-3′ (SEQ ID NO: 4); bFGF, forward 5′-TCAAAGGAGTGTGTGCTAACCG-3′ (SEQ ID NO: 5) and reverse 5′-CTGCCCAGTTCGTTTCAGTG-3′ (SEQ ID NO: 6); and 36B4, forward 5′-GATTGGCTACCCAACTGTTGCA-3′ (SEQ ID NO: 7) and reverse 5′-CAGGGGCAGCAGCCACAAAGGC-3′ (SEQ ID NO: 8). Quantitative real-time PCR was done and analyzed essentially as described (12) with glyceraldehyde-3-phosphate dehydrogenase (GAPDH) for normalization. Primer sequences of HOXB7 for Q-PCR were as follows: forward 5′-AGAGTAACTTCCGGATCTA-3′ (SEQ ID NO: 3) and reverse 5′-CAGGTAGCGATTGTAGTG-3′ (SEQ ID NO: 9) with the TaqMan probe FAM-ACCCCTGGATGCGAAGCTCA-TAMRA (SEQ ID NO: 10) (Applied Biosystems, Foster City, Calif.). Two-tailed Student's t test was used for analysis of the real-time PCR results. Plasmids, siRNA, and transfection. The expression vector for Flag-tagged HOXB7 was a kind gift from Dr. Judith Gasson (University of California at Los Angeles, Los Angeles, Calif.). Wild-type HOXB7 was amplified with primers B7-F (5′-ATGAGTTCATTGTATTATGCGAATG-3′ (SEQ ID NO: 11)) and B7-R (5′-ACTCTTCCTCTTCCTCCTCTGCTTCAG-3′ (SEQ ID NO: 12)) and subcloned into pcDNA3.1-V5-His-Topo vectors (Invitrogen). Flag-tagged HOXB7 and wild-type HOXB7 plasmids were transfected into MCF10A or MDCK cells with Genejammer (Stratagene, La Jolla, Calif.) and cells were selected in 800 μg/mL G418-containing medium to establish stable clones. Two pairs of HOXB7 siRNA oligos [SA1, CAUUCUGUGUGUAUCUAAA (SEQ ID NO: 13) (sense) and UUUAGAUACACACAGAAUG (SEQ ID NO: 14) (antisense); and SA2, GGACUCUCCUUCUGUAAUA (SEQ ID NO: 15) (sense) and UAUUACAGAAUUAGAGUCC (SEQ ID NO: 16) (antisense)] were synthesized and transfected into MDA-MB-435 cells with Lipofectamine 2000 (Invitrogen).

Western blot, immunofluorescence assays, and antibodies. Western blot and immunofluorescence were done as described (13). Antibodies to Flag (Stratagene), HOXB7, claudin 1, claudin 4, claudin 7 (Invitrogen), E-cadherin, vimentin, β-actin (BD Biosciences, Franklin Lakes, N.J.), α-smooth muscle actin (Sigma, St. Louis, Mo.), Ras (Santa Cruz Biotechnology, Santa Cruz, Calif.), MAPK, and phospho-MAPK (Cell Signaling, Danvers, Mass.) were used. Alexa Fluor 488-labeled phalloidin (Invitrogen) was used to detect F-actin.

Wound healing assay. For the wound healing assay, cell cultures at varying confluence were serum starved for 8 hours. After scratching the monolayer, cells were washed with PBS, cultured in 10% fetal bovine serum (FBS)-DMEM, and photographed under 10× objective lens every 3 hours.

Matrigel invasion assay. Into the Biocoat Matrigel invasion chambers (BD Biosciences) were seeded 4×104 cells in 2% FBS medium, and in the lower wells 10% FBS was added as the chemoattractant. After 36 hours of incubation, the filters were stained with crystal violet, and the number of cells that had penetrated through the filter was counted under ×20 magnification (10 randomly selected high-power fields). For inhibition of invasion assays, the FGF receptor inhibitor Su5402 (40 μmol/L), the MAPK/extracellular signal-regulated kinase kinase (MEK) inhibitor U0126 (10 μmol/L), and the Raf inhibitor Bay43-9006 (4 μmol/L; Calbiochem, San Diego, Calif.) were added to the medium in the top chamber at the final concentrations indicated. Ras and RhoA activation assay. Cell lysates were incubated with glutathione S-transferase (GST)-Raf-1 Ras binding domain or GST-Rhotekin RhoA binding domain agarose beads (Upstate, Charlottesville, Va.). GTP-bound Ras or RhoA that precipitated with the beads were detected by SDS-PAGE and immunoblotting with anti-Ras antibody (Santa Cruz Biotechnology) or anti-RhoA antibody (Upstate).

Xenograft analysis. A suspension of 1×106 MDCK-vec or MDCK-B7 cells in 100 μL of Matrigel (BD Biosciences) was injected into the mammary fat pad (one on either side) of 10 female Swiss nu/nu mice. Tumors were removed 8 weeks later and subjected to H&E and immunohistochemical staining for histopathologic examination.

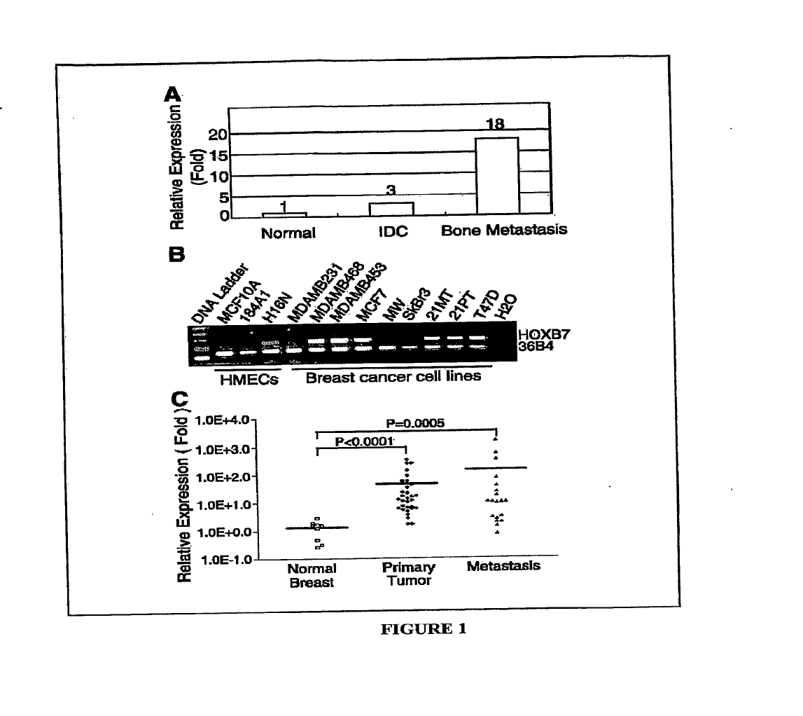

HOXB7 is overexpressed in primary breast carcinoma and metastasis. To identify genes involved in breast cancer progression, oligonucleotide array analysis was done on total RNA isolated from immunobead purified epithelial cells from two normal mammoplasty samples, epithelial cells microdissected from two lymph node-positive primary breast tumors, and three bone metastasis samples. Whereas validation of differentially expressed genes is an ongoing study in our laboratory, our interest was piqued by the 3- and 18-fold overexpression of HOXB7 in the primary and metastatic lesions, respectively, compared with normal mammoplasty samples (FIG. 1A). Because we have previously identified loss of HOXA5 in breast carcinoma (5) and showed its involvement in both p53-dependent and p53-independent apoptotic pathways (5, 6), and accumulating evidence points to a role for HOXB7 in breast cancer, we chose to focus our efforts on this gene.

To validate the microarray data, HOXB7 mRNA was examined by RT-PCR and was found to be expressed in 6 of 10 breast cancer cell lines at higher levels compared with both finite life span and immortalized human mammary epithelial cells (FIG. 1B). These data were further extended and confirmed on 31 primary breast carcinoma samples and 19 metastatic breast lesions by quantitative RT-PCR. HOXB7 mRNA was expressed at higher levels in primary breast carcinomas (P<0.0001) and distant metastasis (P=0.0005), compared with nine purified normal mammary epithelial organoid samples (FIG. 1C). Thus, the results of the microarray analysis were validated in large sample sets of both primary and metastatic breast cancer.

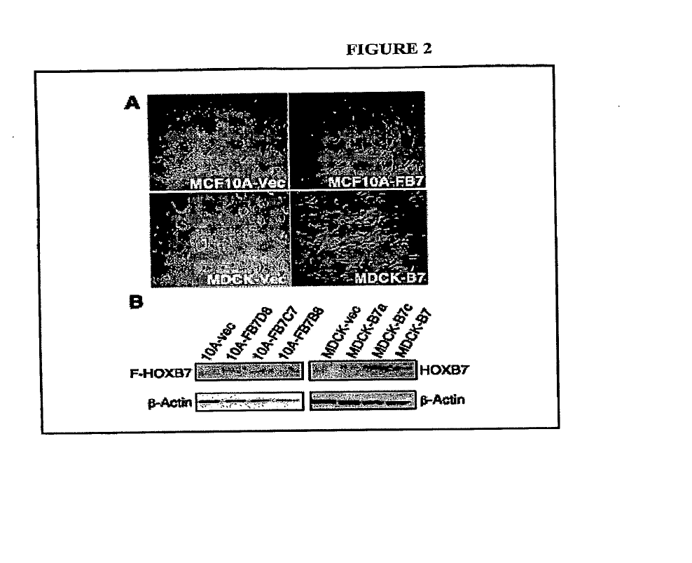

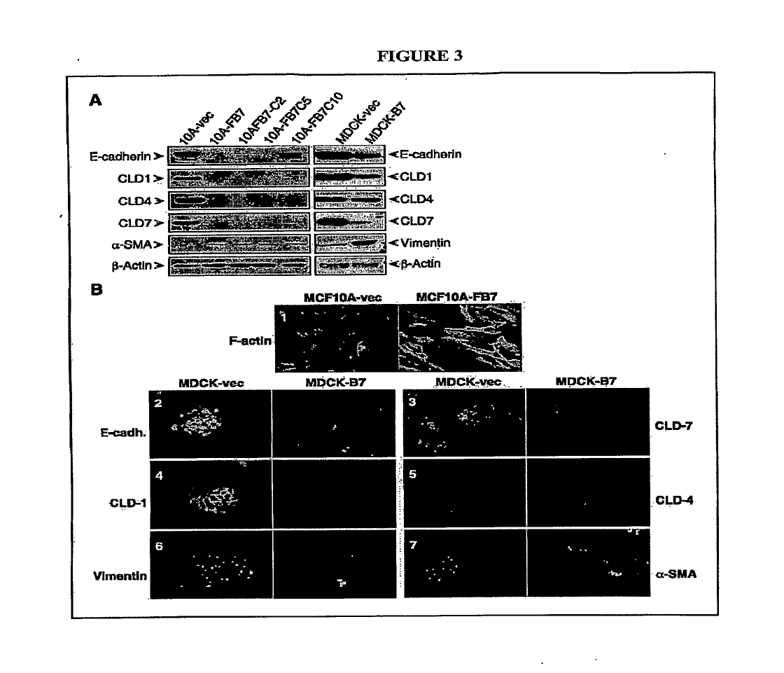

HOXB7 was reported to be present in a novel amplicon on chromosome 17; further fluorescence in situ hybridization (FISH) analysis of 346 tumors showed gene amplification in 10.2% of primary breast cancers, which correlated to poor prognosis (25). We determined if the overexpression of HOXB7 seen in primary tumors can be traced to gene amplification by the use of real-time PCR on tumor DNA for HOXB7 and the internal control gene GAPDH. Our analysis of DNA from the same tumor panel showed that <10% tumors had more than two copies of HOXB7 (data not shown). Thus, gene amplification seems to be the underlying mechanism accounting for only a small percentage of tumors that overexpress HOXB7 mRNA. HOXB7, a novel factor that induces EMT. To determine whether HOXB7 protein expression plays a contributory role in tumor progression, we stably transfected FLAG-tagged HOXB7 (FB7) in MCF10A cells, an immortalized normal mammary epithelial cell line with undetectable HOXB7 expression. Intriguingly, both pooled clones and 70% (16 of 23) of the G418-selected stable clones of MCF10A-FB7 appeared spindle shaped and fibroblastic in monolayer culture, whereas HOXB7-vector control cells, like MCF10A parental cells, maintained their cobblestone-like phenotype (FIG. 2A). This morphologic change implied that the MCF10A-FB7 cells have undergone trans-HOXB7 expression from epithelial cells to mesenchymal cells. Consistent with this observation, rearrangement of cytoskeleton as a signature of the transition was observed by phalloidin staining. In contrast with control cells exhibiting a peripheral F-actin staining with slim central stress fibers, MCF10A-FB7 cells showed a decrease in marginal F-actin but contained much thicker central stress fibers (FIG. 3B-1).

MDCK cells grow as tight islands of cobblestone-shaped cells and have been widely used as a prototypic model to study EMT (26, 27). To determine if the phenotypic change induced in MCF10A cells by HOXB7 is effective in other cell lines, stable clones of MDCK cells expressing HOXB7 were generated. In comparison with the parental and control MDCK cells, >80% clones of MDCK-B7 showed the spindle shape morphology (FIG. 2A). All subsequent experiments were conducted with pools of the MDCK clones. Thus, phenotypic changes typical of EMT were elicited on expression of HOXB7 in both MCF10A and MDCK epithelial cells.

The accepted paradigm of EMT dictates that cells lose markers typical of epithelial cells, such as adhesion molecules (E-cadherin) at the adherens junctions and tight junction proteins (claudins and ZO-1) at the apical junctions (13). Numerous lines of evidence have shown that loss of these proteins impairs cell-cell adhesion and cell-cell communication and facilitates dissemination of metastatic cells (28, 29). Changes in expression of these proteins were examined by Western blot and/or by immunofluorescence analyses. By Western blot analysis, expression of tight junction proteins, claudin 1 and claudin 7, was undetectable in MCF10A-FB7 pooled clones and single clones, and significantly decreased in MDCK-B7 cells (FIG. 3A). Curiously, unlike MCF10A-FB7 cells, expression levels of E-cadherin and claudin 4 were not dramatically changed in MDCK-B7 cells (FIG. 3A). However, by immunofluorescence staining, in contrast to the MDCK control cells where both E-cadherin and claudin-4 proteins had clear membrane peripheral staining pattern, in MDCK-B7 cells their distributions were mainly diffusely cytoplasmic (FIGS. 3B, 2 and 5).

During EMT, loss of epithelial markers is usually accompanied by the expression of markers typical of mesenchymal cells. As shown in FIG. 3B, de novo α-smooth muscle actin expression was observed in MCF10A-FB7 cells, and vimentin expression level was dramatically higher in MDCK-B7 cells (FIG. 3A). In MDCK control and pooled MDCK-HOXB7 cells, although fluorescence signals were observed in both control and HOXB7 transfectants, their expression patterns were completely different. In MDCK control cells, both α-smooth muscle actin and vimentin were localized in a concentrated and polarized pattern. However, in MDCK-B7 cells, α-smooth muscle actin was mainly distributed along the lamellipodia and a network of vimentin intermediate filaments was also clearly visible (FIGS. 3B, 6 and 7). Thus, changes in morphology and molecular markers in both MCF10A and MDCK cells stably expressing HOXB7 were consistent with EMT.

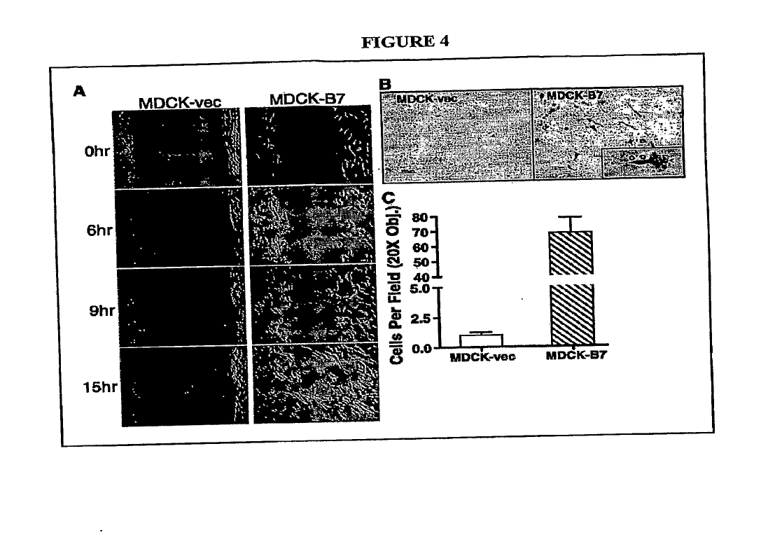

HOXB7 can promote migration and invasion. The essential contribution of EMT to carcinoma progression is that dissociated epithelial cells acquire migration and invasive ability and are able to actively pass through the basement membrane and traverse to distant organs. To test whether HOXB7-overexpressing cells acquire greater migration and invasive ability, two assays were done: the wound healing and the Matrigel invasion assays. The wound healing assay was conducted at different confluence levels of both MDCK-vec and pooled MDCK-B7 cells. FIG. 4A shows representative photomicrographs taken 0, 6, 9, and 15 hours after the cell surface was scratched for the wound healing assay. No motility was observed in MDCK-vec cells during the entire observation period, whereas the pooled MDCK-B7 cells started to fill the wound as early as 6 hours after scratching. Pooled MDCK-B7 cells also showed a significantly greater invasive potential than MDCK-vec cells in the Matrigel invasion assay (FIGS. 4B and C). Moreover, lamellipodia-like structures, an important signature of cell migration, were observed in the majority of pooled MDCK-B7 cells that penetrated and traversed the Matrigel (FIG. 4B).

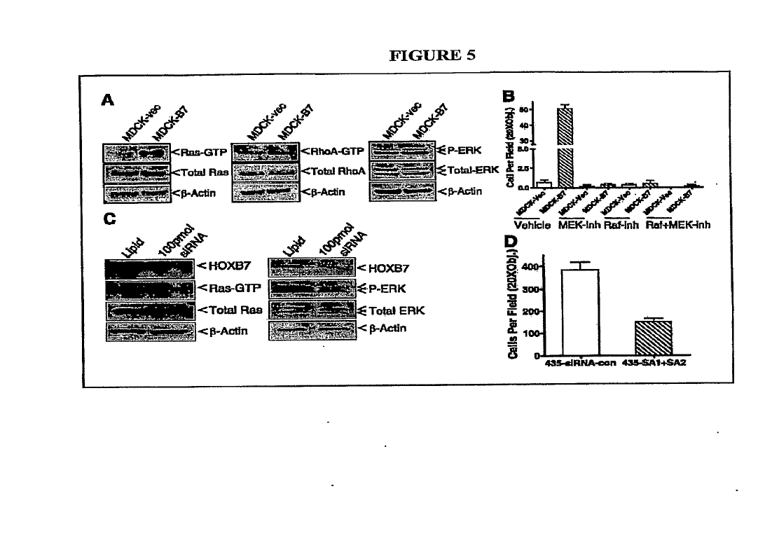

To determine if HOXB7 is involved in promoting invasion of breast epithelial cells, HOXB7 siRNA oligonucleotides, SA1 and SA2, were transiently cotransfected into MDA-MB-435 breast cancer cells that naturally express HOXB7 and exhibit strong invasive ability in vitro. By Western blot analysis, cotransfection of both siRNAs, SA1 and SA2, resulted in the knockdown of HOXB7 expression in MDA-MB-435 cells by 80% to 90% (FIG. 5C). The siRNAs were relatively specific to HOXB7 because no reduction was observed in the expression of two other homeobox genes tested, HOXA5 and HOXD3 (data not shown). The siRNA-transfected MDA-MB-435 cells were then tested for their invasive ability by the Matrigel invasion chamber assay; knockdown of endogenous expression of HOXB7 with specific siRNAs markedly decreased the invasive ability of MDA-MB-435 cells (FIG. 5D).

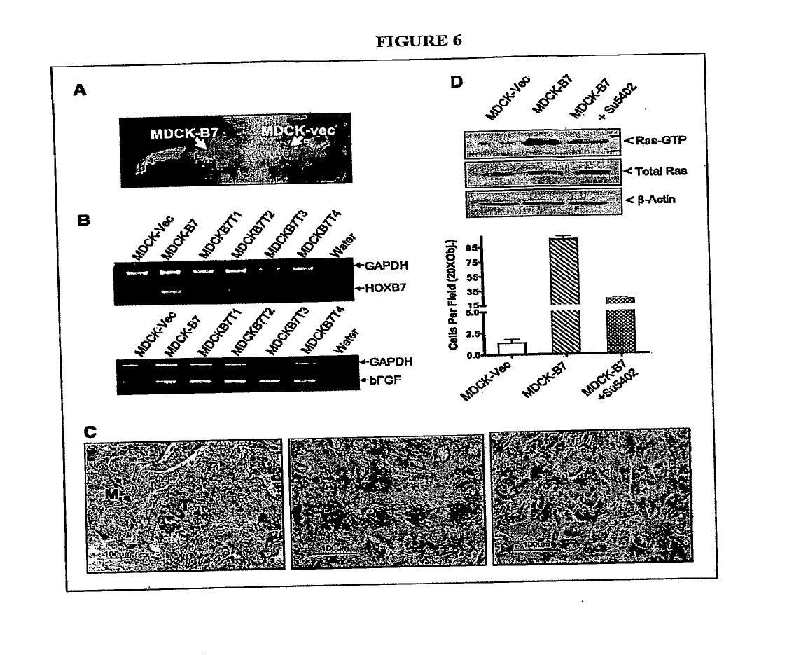

Ras-MAPK pathway is involved in HOXB7 induced EMT. Because it involves scattering of epithelial cells accompanied by a change in morphology to facilitate these movements, EMT is a reflection of the plasticity of differentiated epithelial cells. Multiple signal transduction pathways have been identified to be involved in the induction of EMT. Local expression of growth factors, TGF-β, HGF, EGF/TGF-α, and FGF-2, have been shown to assist EMT by binding to their cognate receptors on epithelial cells and by initiating signal transduction cascades (13). The Ras-Raf-MAPK pathway has been shown to be an indispensable link in the chain of signal transduction leading to induction of EMT (18, 30). To test the involvement of this pathway in EMT induced by HOXB7 in MDCK cells, we employed the GST pull-down assay to analyze the active forms of both RhoA and Ras. In contrast to MDCK-vec cells, pooled clones of MDCK-B7 cells had more active forms of both Ras and RhoA proteins (FIG. 5A). Investigating a role for MAPK activation by testing for p44/p42 MAPK, higher levels of activation of MAPK were observed in pooled MDCK-B7 cells compared with the MDCK-vec cells, with no change in the amount of total protein (FIG. 5A). That this activation is attributable to HOXB7 was tested by suppressing endogenous HOXB7 expression in MDA-MB-435 cells using HOXB7 siRNA. Suppression of HOXB7 expression abrogated activation of the Ras-MAPK pathway (FIG. 5C), suggesting a key role for HOXB7 in this phenomenon. Further confirmation was sought that activation of these pathways is associated with the invasive ability of MDCK-B7 cells. MDCK-vec and pooled MDCK-B7 cells were seeded into Matrigel invasion chambers and treated with the RAF inhibitor Bay43-9006 or the MEK inhibitor U0126. Treatment of the cells with either inhibitor alone or in combination resulted in the complete suppression of the invasive phenotype displayed by the MDCK-B7 cells (FIG. 5B). Similar results were observed in migration assays (data not shown). bFGF, a wide-spectrum factor functioning in mitogenesis (31), angiogenesis (32), and neurogenesis (33), was previously reported as a directly regulated gene target of HOXB7 (34). bFGF has also been shown to induce EMT in lens cells (35) in response to injury and in kidney cells (36). Furthermore, activation of FGF receptors by autocrine bFGF results in the recruitment and phosphorylation of adaptor protein SHC, which then creates binding sites for the growth factor receptor binding protein-2 adaptor in complex with the Ras-activating nucleotide exchange factor SOS (37). To verify whether this signal transduction pathway is involved in the HOXB7-induced invasive properties in our system, we first tested for bFGF expression by RT-PCR in both pooled MDCK-B7 cells and MDCK-B7 xenograft tumors grown in nude mice. FIG. 6B shows that bFGF was expressed at higher levels in both MDCK-B7 cells and transplanted tumors compared with MDCK-vector control cells. Treatment of MDCK-B7 cells with the FGF receptor-specific inhibitor Su5402 (Calbiochem) could attenuate the Ras-GTP form (FIG. 6D). Further, the FGF receptor-specific inhibitor could inhibit invasion ability of MDCK-B7 cell by ˜80% (FIG. 6D). The expression of some other well-known EMT regulators, such as members of the TGF-β pathway and members of the Snail family, was also determined by RT-PCR or Western blot analysis, but no significant change was detected (data not shown). Thus, several lines of evidence point to bFGF as the major mediator of EMT initiated by HOXB7.

Overexpression of HOXB7 in MDCK cells promotes tumor formation and local invasion in vivo. All the in vitro culture data indicated that the expression of HOXB7 conferred many features of neoplastic transformation to MDCK cells. To test whether MDCK-B7 cells were tumorigenic in vivo, MDCK-vec and pooled MDCK-B7 cells were implanted as Matrigel plugs into the mammary fat pads of immunodeficient female nude mice. Tumors formed in nine of the ten sites injected with MDCK-B7 cells (FIG. 6A), but no distant metastases were found in tumor-bearing mice. RT-PCR confirmed that HOXB7 expression was retained in the implanted tumors (FIG. 6B). In contrast, no palpable tumors were observed in any of the sites injected with MDCK-vec cells. MDCK-B7 tumors were firmly attached to the surrounding tissues, including the underlying axillary muscle. Histopathologic examination showed that the tumors lacked well-defined capsular margins; tumor cells had infiltrated into the surrounding tissues; and islands of tumor cells dissociated from the main tumor mass were also observed (FIG. 6C). Immunohistochemical examination of tumor sections with a marker of proliferation, Ki67, and of vascular endothelial cells, CD146, revealed that the tumors were proliferative and highly vascularized. Thus, HOXB7-overexpressing MDCK cells were tumorigenic and formed aggressive invasive tumors that were well vascularized.

Metastasis accounts for the majority of breast cancer-related mortality, and bone marrow is one the most favored metastatic sites for breast cancer. In this study, we compared gene expression profiles from purified normal and tumor epithelial cells isolated from normal mammoplasty tissues, primary invasive breast tumor, and bone metastasis. HOXB7, a homeobox gene, showed a stepwise increase in expression during tumor progression. EMT was originally recognized as a step to metazoan embryogenesis and in defining structures during organ development (38). During the last decade, a number of studies have related EMT to cancer progression and, in parallel, a role for HOX genes in cancer. In this article, we show that HOXB7 conferred EMT to epithelial cells, with a gain of biological features consistent with neoplastic transformation and invasiveness. Overexpression of HOXB7 in mammary epithelial cells, MCF10A, and a prototypical model epithelial cell line, MDCK, could induce the conversion of cobblestone-like epithelial morphology to spindle-shape mesenchymal morphology. Consistent with the morphologic change, some hallmark proteins of epithelial cells were lost or reduced during the transition. Intriguingly, in MDCK-B7 cells, instead of a dramatic change in expression levels, the epithelial-specific proteins, E-cadherin and claudin 4, altered their localization from the cell membrane to the cytoplasm. A similar E-cadherin translocation pattern was documented by Bellovin et al. in 2005 (39), where they also showed that cytoplasmic localization of E-cadherin was correlated to EMT and genesis of metastasis of colorectal tumors. These observations suggest that although expression levels of these adhesion and tight junction proteins were not significantly altered, improper subcellular localization could result in protein loss of function and contribute to progression of metastasis. On the other hand, we only observed this phenomenon in MDCK-B7 cells, but not in MCF10A-B7 cells. This implies that the mechanism of EMT induction by HOXB7 could be cell context dependent.

These morphologic and cell-cell contact changes ultimately reflect on cell mobility and invasive ability. It is well known that small GTP binding proteins, such as members of the Ras and Rho families, comprehensively regulate cell migration and invasion. Also well documented is that the Ras-RAF-MAPK pathway plays indispensable role in EMT induced by activation of receptor tyrosine kinase of growth factors like HGF, VEGF, EGF, and bFGF (30). Taking into account published findings that HOXB7 could directly transactivate the expression of bFGF in both melanoma and breast cancer cell lines (23, 34), we investigated bFGF expression and found that it was high in both HOXB7 stably transfected MDCK cells and the xenograft tumors. Further, blocking FGF autocrine signaling cascade with the FGF receptor inhibitor Su5402 could attenuate activation of the Ras-RAF-MAPK pathway and the invasive ability of MDCK-B7 cells. In addition to activation of the Ras pathway, more RhoA-GTP form proteins were observed in HOXB7-transfected cells. RhoA protein is known by its ability to remodel the actin cytoskeleton and form thick stress fibers, which are required for migratory behavior of cells (40). Using MDCK cells, Zondag et al. (27) have shown that a shift in balance between RhoA and RAC activity can control the transition of phenotype from epithelial to mesenchymal. They found that sustained signaling by oncogenic RasV12 permanently down-regulated RAC activity, which led to up-regulation of RhoA activity and EMT. On the other hand, reconstitution of RAC activity by expression of Tiam1 or RACV12 led to down-regulation of Rho activity and restored an epithelial phenotype to mesenchymal, RasV12-transformed cells (27). Although activation of RhoA mediates formation of F-actin stress fibers and enhances cell motility, some studies indicate that excessive activation of RhoA can actually inhibit polarization and motility (41, 42). For example, stimulation of U118 cells with SIP resulted in a 5-fold induction of RhoA activity and inhibition of migration (42). In the case of HOXB7-transformed MDCK cells, higher bFGF expression was observed compared with vector controls (FIG. 6B). The expression of bFGF could result in constitutively activating Ras signaling through autocrine signaling cascades. Activated Ras signaling pathways could further activate RhoA by ˜2-fold (FIG. 5A), which could have mediated the formation of contractile stress fiber to facilitate migration and invasion.

### REFERENCES

1. Lechner J F, Fugaro J M, Wong Y, Pass H I, Harris C C, Belinsky S A. Perspective: cell differentiation theory may advance early detection of and therapy for lung cancer. Radiat Res 2001; 155:235-8.
2. Miller G J, Miller H L, van Bokhoven A, et al. Aberrant HOXC expression accompanies the malignant phenotype in human prostate. Cancer Res 2003; 63:5879-88.
3. Naora H, Yang Y Q, Montz F J, Seidman J D, Kurman R J, Roden R B. A serologically identified tumor antigen encoded by a homeobox gene promotes growth of ovarian epithelial cells. Proc Natl Acad Sci USA 2001; 98:4060-5.
4. Yoshida H, Broaddus R, Cheng W, Xie S, Naora H. Deregulation of the HOXA10 homeobox gene in endometrial carcinoma: role in epithelial-mesenchymal transition. Cancer Res 2006; 66:889-97.
5. Raman V, Martensen S A, Reisman D, et al. Compromised HOXA5 function can limit p53 expression in human breast tumours. Nature 2000; 405:974-8.
6. Chen H, Chung S, Sukumar S. HOXA5-induced apoptosis in breast cancer cells is mediated by caspases 2 and 8. Mol Cell Biol 2004; 24:924-35.
7. Carrio M, Arderiu G, Myers C, Boudreau N J. Homeobox D10 induces phenotypic reversion of breast tumor cells in a three-dimensional culture model. Cancer Res 2005; 65:7177-85.
8. Ma X J, Wang Z, Ryan P D, et al. A two-gene expression ratio predicts clinical outcome in breast cancer patients treated with tamoxifen. Cancer Cell 2004; 5:607-16.
9. Greenburg G, Hay E D. Epithelia suspended in collagen gels can lose polarity and express characteristics of migrating mesenchymal cells. J Cell Biol 1982; 95:333-9.
10. Moody S E, Perez D, Pan T C, et al. The transcriptional repressor Snail promotes mammary tumor recurrence. Cancer Cell 2005; 8:197-209.
11. Kang Y, Massague J. Epithelial-mesenchymal transitions: twist in development and metastasis. Cell 2004; 118:277-9.
12. Petersen O W, Nielsen H L, Gudjonsson T, et al. Epithelial to mesenchymal transition in human breast cancer can provide a nonmalignant stroma. Am J Pathol 2003; 162:391-402.
13. Thiery J P. Epithelial-mesenchymal transitions in tumour progression. Nat Rev Cancer 2002; 2:442-54.
14. Mironchik Y, Winnard P T, Jr., Vesuna F, et al. Twist overexpression induces in vivo angiogenesis and correlates with chromosomal instability in breast cancer. Cancer Res 2005; 65:10801-9.
15. Yang J, Mani S A, Donaher J L, et al. Twist, a master regulator of morphogenesis, plays an essential role in tumor metastasis. Cell 2004; 117:927-39.
16. Cowin P, Rowlands T M, Hatsell S J. Cadherins and catenins in breast cancer. Curr Opin Cell Biol 2005; 5:499-508.
17. Balkovetz D F, Pollack A L, Mostov K E. Hepatocyte growth factor alters the polarity of Madin-Darby canine kidney cell monolayers. J Biol Chem 1997; 272:3471-7.
18. Janda E, Lehmann K, Killisch I, et al. Ras and TGFB cooperatively regulate epithelial cell plasticity and metastasis: dissection of Ras signaling pathways. J Cell Biol 2002; 156:299-313.
19. Lu Z, Ghosh S, Wang Z, Hunter T. Down-regulation of caveolin-1 function by EGF leads to the loss of E-cadherin, increased transcriptional activity of β-catenin, and enhanced tumor cell invasion. Cancer Cell 2003; 4:499-515.
20. Radisky D C, Levy D D, Littlepage L E, et al. Rac1b and reactive oxygen species mediate MMP-3-induced EMT and genomic instability. Nature 2005; 436:123-7.
21. Davidson N E, Sukumar S. Of Snail, mice, and women. Cancer Cell 2005; 8:173-4.
22. Srebrow A, Friedmann Y, Ravanpay A, Daniel C W, Bissell M J. Expression of Hoxa-1 and Hoxb-7 is regulated by extracellular matrix-dependent signals in mammary epithelial cells. J Cell Biochem 1998; 69:377-91.
23. Care A, Silvani A, Meccia E, Mattia G, Peschle C, Colombo M P. Transduction of the SkBr3 breast carcinoma cell line with the HOXB7 gene induces bFGF expression, increases cell proliferation and reduces growth factor dependence. Oncogene 1998; 16:3285-9.
24. Care A, Felicetti F, Meccia E, et al. HOXB7: a key factor for tumor-associated angiogenic switch. Cancer Res 2001; 61:6532-9.
25. Hyman E, Kauraniemi P, Hautaniemi S, et al. Impact of DNA amplification on gene expression patterns in breast cancer. Cancer Res 2002; 62:6240-5.
26. Stoker M, Perryman M. An epithelial scatter factor released by embryo fibroblasts. J Cell Sci 1985; 77:209-23.
27. Zondag G C, Evers E E, ten Klooster J P, Janssen L, van der Kammen R A, Collard J G. Oncogenic Ras down-regulates Rac activity, which leads to increased Rho activity and epithelial-mesenchymal transition. J Cell Biol 2000; 149:775-82.
28. Kominsky S L, Argani P, Korz D, et al. Loss of the tight junction protein claudin-7 correlates with histological grade in both ductal carcinoma in situ and invasive ductal carcinoma of the breast. Oncogene 2003; 22:2021-33.
29. Maeda M, Johnson K R, Wheelock M J. Cadherin switching: essential for behavioral but not morphological changes during an epithelium-to-mesenchyme transition. J Cell Sci 2005; 118:873-87.
30. Kalluri R, Neilson E G. Epithelial-mesenchymal transition and its implications for fibrosis. J Clin Invest 2003; 112:1776-84.
31. Riboni L, Viani P, Bassi R, Giussani P, Tettamanti G. Basic fibroblast growth factor-induced proliferation of primary astrocytes. evidence for the involvement of sphingomyelin biosynthesis. J Biol Chem 2001; 276:12797-804.
32. Cao R, Brakenhielm E, Pawliuk R, et al. Angiogenic synergism, vascular stability and improvement of hind-limb ischemia by a combination of PDGF-BB and FGF-2. Nat Med 2003; 9:604-13.
33. Gritti A, Parati E A, Cova L, et al. Multipotential stem cells from the adult mouse brain proliferate and self-renew in response to basic fibroblast growth factor. J Neurosci 1996; 16:1091-100.
34. Care A, Silvani A, Meccia E, et al. HOXB7 constitutively activates basic fibroblast growth factor in melanomas. Mol Cell Biol 1996; 16:4842-51.
35. Lee H T, Kay E P. FGF-2 induced reorganization and disruption of actin cytoskeleton through PI 3-kinase, Rho, and Cdc42 in corneal endothelial cells. Mol V is 2003; 9:624-34.
36. Strutz F, Zeisberg M, Ziyadeh F N, et al. Role of basic fibroblast growth factor-2 in epithelial-mesenchymal transformation. Kidney Int 2002; 61:1714-28.
37. Kouhara H, Hadari Y R, Spivak-Kroizman T, et al. A lipid-anchored Grb2-binding protein that links FGF-receptor activation to the Ras/MAPK signaling pathway. Cell 1997; 89:693-702.
38. Lee J M, Dedhar S, Kalluri R, Thompson E W. The epithelial-mesenchymal transition: new insights in signaling, development, and disease. J Cell Biol 2006; 172:973-81.
39. Bellovin D I, Bates R C, Muzikansky A, Rimm D L, Mercurio A M. Altered localization of p120 catenin during epithelial to mesenchymal transition of colon carcinoma is prognostic for aggressive disease. Cancer Res 2005; 65:10938-45.
40. Malliri A, Collard J G. Role of Rho-family proteins in cell adhesion and cancer. Curr Opin Cell Biol 2003; 15:583-9.
41. Xu J, Wang F, Van Keymeulen A, et al. Divergent signals and cytoskeletal assemblies regulate self-organizing polarity in neutrophils. Cell 2003; 114:201-14.
42. Lepley D, Paik J H, Hla T, Ferrer F. The G protein-coupled receptor S1P2 regulates Rho/Rho kinase pathway to inhibit tumor cell migration. Cancer Res 2005; 65:3788-95.

### Example 2

Homeobox genes encode transcription factors which function in body axis patterning in the developing embryo. Recent evidence suggests that the maintenance of specific HOX expression patterns is necessary for regulating the homeostasis of adult tissues as well. In this study, HOXB7 transformed human mammary epithelial cells, MCF10A, to grow in minimally supplemented medium, to form colonies in Matrigel, and display resistance to ionizing radiation. Searching for protein partners of HOXB7 that might contribute to resistance to ionizing radiation, we identified four HOXB7-binding proteins by GST pull-down/affinity chromatography and confirmed their interactions by coimmunoprecipitation in vivo. Interestingly, all four HOXB7-binding proteins shared functions as genomic caretakers and included members of the DNA-dependent protein kinase holoenzyme (Ku70, Ku80, DNA-PKcs) responsible for DNA double-strand break repair by nonhomologous end joining pathway and poly(ADP) ribose polymerase. Exogenous and endogenous expression of HOXB7 enhanced nonhomologous end joining and DNA repair functions in vitro and in vivo, which were reversed by silencing HOXB7. This is the first mechanistic study providing definitive evidence for the involvement of any HOX protein in DNA double-strand break repair. [Cancer Res 2007; 67(4):1527-35]

HOX genes encode transcription factors that are characterized by a highly conserved trihelical homeodomain that binds to specific DNA sequences. A total of 39 HOX genes have been identified that are organized into four paralogous clusters, HOX-A to HOX-D, on autosomal chromosomes (1). The functions of homeodomain-containing proteins are diverse and include roles as both classical regulators of transcription and novel roles outside of transcriptional regulation. HOX genes are functionally important in anteroposterior patterning during embryogenesis, homeostasis in adult tissue, cell to cell interactions, and cell to extracellular matrix interactions (reviewed in ref. 2). Examples of novel roles for homeodomain-containing proteins include the role of human proline-rich homeodomain protein, PRH (known as Hex in studies on hematopoiesis), which interacts with eIF4E to inhibit its mRNA nuclear-cytoplasmic transport function (3). Given that HOX proteins can bind to very similar sequences in vitro but exert diverse functions in vivo, a fundamental question is how each HOX protein achieves functional specificity. One hypothesis is that functional specificity is attained by physical interaction with various cofactors.

DNA double-strand breaks (DSB), caused by exposure to ionizing radiation (IR), certain chemicals, or occurring during replication, V(D)J recombination, and meiosis, pose a major challenge to the maintenance of genomic integrity. If they are left unrepaired, cell cycle arrest, apoptosis, or mitotic cell death ensues, whereas faulty repair can lead to neoplastic transformation (4, 5). Nonhomologous end joining (NHEJ) is the major mechanism for the repair of IR-induced DSB, and involves the DNA end-binding heterodimer, Ku70/Ku80, the DNA-dependent protein kinase (DNA-PK), the XRCC gene product, and DNA ligase IV (6). The Ku antigen binds to and recruits DNA-PK to sites of DNA strand breaks, where DNA-PK is activated to participate in DNA repair. HOXC4 and HOXD4, along with homeodomain-containing proteins Octamer transcription factors 1 and 2, and Dlx2, interact with the COOH terminus of the Ku antigen causing their recruitment to broken DNA ends and phosphorylation by DNA-PK (7). However, the functional significance of this interaction is not known.

Another protein that contributes to genomic stability is poly(ADP) ribose polymerase (PARP). PARP catalyzes the transfer of polymers of ADP-ribose from NAD+ onto protein targets (8, 9), and regulates both cell survival and cell death programs. A recent study has shed some light on their involvement in DSB repair mediated by NHEJ and by homologous recombination (HR). Hochegger et al. (8) showed that PARP-1(−/−) mutant chicken cells have reduced levels of HR and are sensitive to various DSB-inducing genotoxic agents. Interestingly, this phenotype is strictly dependent on the presence of Ku70. PARP-1/KU70 double mutants are proficient in the execution of HR and display an elevated resistance to DSB-inducing drugs. These results suggest that PARP might function by minimizing the suppressive effects of Ku and the NHEJ pathway on HR.

It was found that HOXB7 has the ability to confer both a transformed phenotype and resistance to IR in cultures of human mammary epithelial cells (HMEC), MCF10A. A search for protein interaction partners for HOXB7 that might contribute to this transformation led to the identification of the DNA repair proteins, Ku70, Ku80, the catalytic subunit of DNA-PK (DNA-PKcs), and PARP. This, among other functions, suggests a role for HOXB7 in DNA repair through NHEJ. Evidence indicates that interaction between HOXB7 and the Ku antigens is functionally significant because HOXB7 expression enhances NHEJ, DNA-PK activity, and DNA damage repair in mammalian cells.

Cell culture, plasmids, transfections, and antibodies. Breast cancer cell lines were obtained from the American Type Culture Collection (Manassas, Va.) and cultured as follows: SKBR3 cells in McCoy's 5A medium containing 15% fetal bovine serum (FBS), MDA-MB-231, MDA-MB-468, and MCF-7 cells in DMEM supplemented with 10% FBS. HMECs, MCF10A, and MCF12A, were cultured as described (10). Chinese hamster ovary (CHO) cells were cultured in DMEM/F-12 medium containing 10% FBS. All plasmids were sequenced to verify fidelity. FLAG-tagged or green fluorescent protein (GFP)-tagged HOXB7 or vector-transfected cells were selected in 800 μg/mL of G418-containing medium and cell clones were analyzed for expression of the fusion protein by Western blot and fluorescence microscopy. Expression vectors for Fl-tagged HOXB7 and mutants have been previously described (11). Transfections were done using Genejammer (Stratagene, La Jolla, Calif.). Transfection in MCF10A cells was done using Effectene (Qiagen, Valencia, Calif.) according to the manufacturer's instructions. His-tagged Ku70 and Ku80 expression plasmids were provided by Dr. Kathrin Muegge (National Cancer Institute, Bethesda, Md.) and PARP-pCR3.1 was provided by Dr. Solomon Snyder (Johns Hopkins University School of Medicine, Baltimore, Md.). SDS-PAGE and Western blots were done as described (12). The following monoclonal antibodies were used for protein detection by immunoblot: anti-FLAG M2 (Sigma, St. Louis, Mo.), PARP (clone C-2-10; Invitrogen, Carlsbad, Calif.), DNA-PKcs (clone G-4; Santa Cruz Biotechnology, Santa Cruz, Calif.), Ku70 (clone 2C3.11; Novus Biologicals, Littleton, Colo.), Ku86 (clone B-1; Santa Cruz Biotechnology), Living Colors A.v. (clone JL-8; Clontech, Mountain View, Calif.) for detection of YFP and HOXB7-YFP, and GST goat polyclonal antibody (GE Healthcare, Piscataway, N.J.).

Cell proliferation assays. MCF10A cells stably expressing HOXB7-Fl or vector control cells were grown in RPMI supplemented with 1% or 10% FBS, adherent cells were fixed in 10% formalin for 20 min, stained with 0.1% crystal violet, and lysed in 10% acetic acid. Colorimetric measurements were done using a microplate reader (Molecular Devices, Sunnyvale, Calif.) at 590 nm. Measurements were done in triplicate and the experiment was repeated thrice. Growth in and on Matrigel was assessed as described in ref. 13. Colony formation in Matrigel was assessed after 1 week. The number of colonies containing >200 cells was counted. Cells grown on Matrigel were assessed for the formation of three-dimensional structures 3 weeks after seeding. The morphology of the structures formed using MCF10A-vec and those formed by MCF10A-Fl-HOXB7 cells were compared and photographed under phase contrast at 20× magnification.

GST-HOXB7 affinity chromatography and identification of GST-HOXB7-binding proteins. GST-HOXB7 was expressed as previously described (11). The GST and GST-PRL3 expression plasmids were provided by Dr. Bert Vogelstein (Johns Hopkins University School of Medicine). Quantitation of GST or GST fusion proteins was done by silver staining SDS-PAGE gels using bovine serum albumin as a standard. Soluble fusion proteins, used as controls on protein gels, were eluted from the Sepharose beads with 25 mmol/L of glutathione (Sigma)/PBS (pH 8.0). Cell protein extracts were prepared from SKBR3, MCF10A, MCF-12A, MDA-MB-231 by scraping cells in 500 μL of EBC lysis buffer [50 mmol/L Tris-HCl (pH 8.0), 120 mmol/L NaCl, 0.5% NP40] supplemented with complete protease inhibitor cocktail (Roche, Indianapolis, Ind.). All cell extracts were precleared by prior incubation for 1 h with 5 μg of GST-Sepharose. For affinity chromatography, 5 μg (50-100 μL) of GST-HOXB7-Sepharose or control fusion protein was mixed for 2.5 h at 4° C. with 5 mg of cell protein extracts. The beads were washed five times with 1 mL of EBC cell lysis buffer and eluted in 25 mmol/L glutathione/PBS (pH 8.0). Eluates were divided into two aliquots for protein staining or Western blot following SDS-PAGE. Protein identities were determined by one of two methods: direct sequencing from Coomassie blue-stained polyvinylidene difluoride (PVDF) membranes or peptide mass fingerprinting from tryptic peptides of Coomassie blue-stained bands on the gel, both done at the Stanford PAN facility (Palo Alto, Calif.). All protein identifications were confirmed by immunoblotting with corresponding antibodies.

Communoprecipitation. For coimmunoprecipitation of HOXB7-binding proteins from SKBR3 cells expressing HOXB7-YFP or Fl-tagged HOXB7, 1 to 2 mg of cell protein extracts prepared as described above, were precleared (12) and subjected to immunoprecipitation for 2.5 h at 4° C. with the following antibodies: full-length A.v. polyclonal antibodies (Clontech) for immunoprecipitation of HOXB7-YFP, or anti-FLAG polyclonal antibodies (Sigma) for precipitation of FL-HOXB7 complexes according to the suggestions of the manufacturer. Complementary coimmunoprecipitation of HOXB7-YFP with its binding proteins was done with monoclonal antibodies to human DNA-PKcs and human Ku86 (clones H-163 and C-20, respectively; Santa Cruz Biotechnology) as described (12). To verify the interaction between endogenous HOXB7 with Ku70, Ku80, and DNA-PK under physiologic conditions and the effect of DNA depletion, 1 to 2 mg of whole cell lysates of MCF-7 (with or without ethidium bromide) were subjected to immunoprecipitation with Ku70 and Ku80 antibodies, the immune complexes were loaded onto 4% to 12% NuPAGE gels (Invitrogen) and immunoblotted with anti-HsKu70 (clone 2C3.11; Novus Biologicals), Ku86 (C-20; Santa Cruz Biotechnology), or anti-HOXB7 rabbit polyclonal antibodies (Invitrogen).

DNA repair and cell survival assays. Plasmid end-joining assays were done essentially as described in ref. 14. Briefly, nuclear extracts of SKBR3-HOXB7-YFP or vector-transfected cells were prepared with NE-PER Reagent (Pierce, Rockford, Ill.). Four micrograms of nuclear extracts were mixed with 0.25 μg of EcoRV- or BamHI-cut pcDNA3, and digested for 1 h at 25° C. in a buffer containing 20 mmol/L of Hepes-KOH (pH 7.5), 10 mmol/L of MgCl2, and 80 mmol/L of KCl. The reaction was stopped with the addition of 2 μL of 5% SDS, 2 μL of 0.5 mol/L EDTA, and 1 μL of 10 mg/mL proteinase K and incubation at 37° C. Half of each reaction was resolved by electrophoresis on agarose gels. UV detection and densitometric quantitation was done using EagleEye Software. All experiments were done in duplicate and repeated twice. Relative NHEJ activity was obtained by calculating mean densitometric units of all the end-joined products on the gel.

Cell survival following gamma irradiation, measurements of mitotic indexes and determination of G1 and G2-type chromosomal aberrations after DNA damage. These experiments were done as described in ref. 15. Cells in plateau phase were irradiated with 3 Gy, subcultured, and examined for G1-type aberrations at metaphase. All categories of asymmetric chromosome aberrations were scored: dicentrics, centric rings, interstitial deletions/acentric rings, and terminal deletions. For G2-type aberrations, cells in exponential phase growth were irradiated with 1 Gy gamma irradiation. Metaphases were harvested 45 and 90 min following irradiation and examined for chromatid breaks and gaps. Fifty metaphases were scored for each postirradiation time point.

DNA-PK assay. Cell extracts were prepared as follows: MDA-MB-435 cells, cultured as monolayers, were harvested using a cell scraper, washed twice in PBS, snap-frozen on dry ice and stored at −80° C. Frozen cell pellets were resuspended in 70 to 90 μL of hypotonic lysis buffer [10 mmol/L Tris (pH 8.0), 1 mmol/L EDTA], incubated on ice for 20 min, and then subjected to vigorous vortexing for 30 s. High salt buffer [83.5 mmol/L Tris (pH 8.0), 1.65 mol/L KCl, 3.3 mmol/L EDTA, 1 mmol/L DTT] was added to 20% of total volume followed by incubation on ice for 20 min. Cell debris was removed by centrifugation (16,500×g, 10 min, 4° C.) and the resulting supernatant was collected as an extract. KCl was added to a final concentration of 0.5 mol/L, 25 μL of DEAE Sepharose resin (GE Healthcare) was added to remove DNA and the sample was rotated for 30 min at 4° C. DEAE Sepharose was removed by centrifugation and the sample was dialyzed against 20 mmol/L of Tris (pH 8.0), 0.1 mol/L of KHAc, 10% glycerol, 0.5 mmol/L of EDTA, and 1 mmol/L of DTT. DNA-PK assays were done in duplicate according to the instructions of the manufacturer (Promega, Madison, Wis.) using 40 μg of the extract/assay. Three separate assays were done. The results were calculated as mean±SD. Two-tailed Student's t test was done to calculate P values.

Small interfering RNA expression construct and transfection. The small interfering RNA (siRNA) sequences used for targeting human HOXB7 were 5′-ATATCCAGCCTCAAGTTCG-3′ (SEQ ID NO: 1) and 5′-ACTTCTTGTGCGTTTGCTT-3′ (SEQ ID NO: 2). Oligonucleotides encoding siRNAs (Invitrogen) were annealed and ligated into pSilencer-U6 vector (Genscript, Piscataway, N.J.). The two HOXB7 siRNA expression plasmids were mixed 1:1 for transfection. Plasmids (1 μg/well) containing HOXB7 siRNA or siRNA of the scrambled sequence (control) was transfected into six-well plates by use of Effectene (Qiagen) for 24 h.

Measurement of DNA DSBs. Assay of DNA DSB repair activity following DNA damage induced by IR was done under nondenaturing conditions by a standard procedure using pulsed-field gel electrophoresis (PFGE) as described previously (16, 17). Cells kept on ice received a gamma radiation dose of 50 Gy. Immediately following irradiation, the cells were placed in medium at 37° C., incubated at 37° C. for various time periods, trypsinized, washed, and embedded in agarose plugs, lysed, and digested with proteinase K. Plugs were washed in TE buffer [10 mmol/L Tris-HCl, 1 mmol/L Na2 EDTA (pH 8)] and PFGE was carried out with a contour-clamped homogeneous electric field in 0.8% agarose gels. The gels were run at 14° C. with linearly increasing pulse times as described (16, 17). Gels were stained with ethidium bromide and photographed with a charge-coupled device camera system under UV transillumination. Quantitative analysis to determine the fraction of DNA entering the gel provided a measure for the relative number of DSBs. The control cell DNA was normalized to zero and 100% was assigned to DNA of cells treated with 50 Gy with no repair.

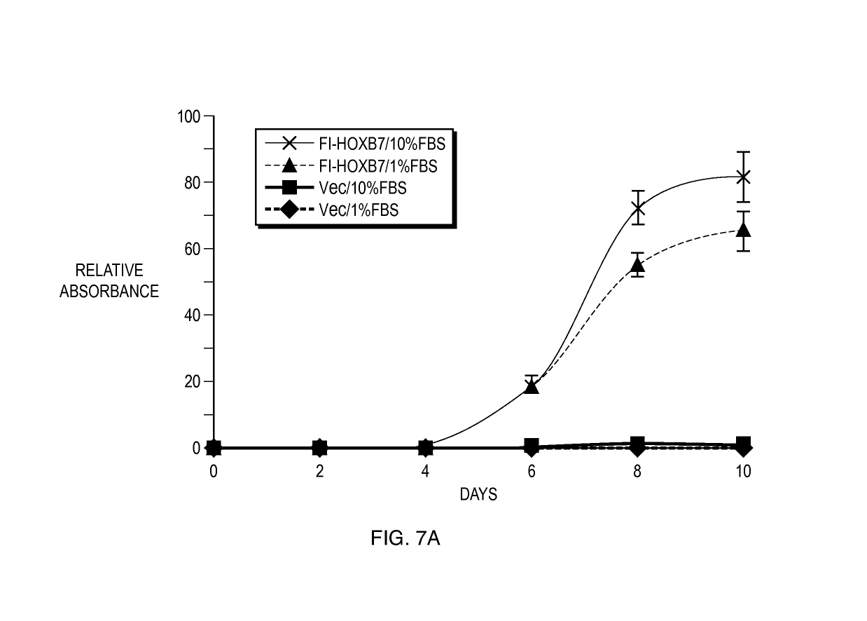

HOXB7 elicits a transformed phenotype in MCF10A cells. Overexpression of HOXB7 enabled nontumorigenic breast cancer cells, SKBR3, to form well-vascularized tumors in immunodeficient mice (18). To investigate whether HOXB7 expression transforms normal breast epithelial cells, a FLAG-tagged (Fl) HOXB7 expression plasmid was transfected into immortalized HMECs, MCF-10A, and pooled G418-selected colonies stably expressing HOXB7 were tested for alterations in growth properties compared with the vector-transfected cells (FIGS. 7A and B). MCF10A cells require a highly growth factor-supplemented medium for optimal growth (19). In low growth nutrient RPMI with 10% or 1% serum supplementation, unlike vector-control cells, MCF10A-Fl-HOXB7 cells displayed a continued ability to proliferate (FIG. 7A). Grown in Matrigel in complete medium, in contrast to the minute colonies formed by MCF-10A-vec-transfected cells (FIG. 7C, 1), MCF10A-Fl-HOXB7 cells formed 200 to 300 cells, anchorage-independent colonies (FIG. 7C, 2) within 3 weeks. Grown on Matrigel-coated plates, MCF10A cells formed discrete acini-like structures with a hollow lumen (FIG. 7C, 3), whereas MCF10A-Fl-HOXB7 cells displayed large irregular solid colonies with cells pushing haphazardly into the surrounding extracellular matrix (FIG. 7C, 4). Taken together, the results indicate that the MCF10A-Fl-HOXB7 cells exhibit a transformed phenotype like that of MCF10A cells expressing RAS or HER2/neu oncogenes (13, 19).

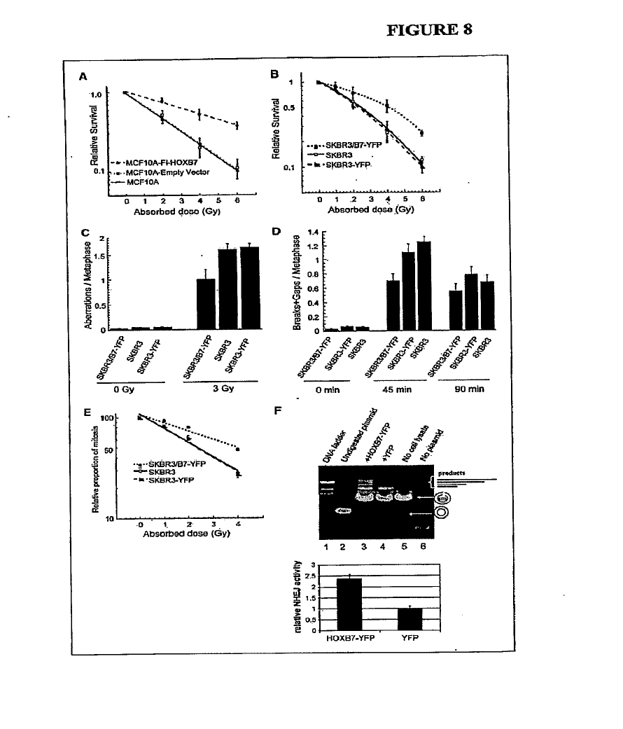

HOXB7 increases resistance to IR. Some activated oncogenes render cells resistant to radiation whereas others enhance their susceptibility to IR. The molecular basis of sensitivity to IR is a complex product of cellular responses; loss of cell cycle checkpoints may result in increased sensitivity, particularly if the checkpoint controls G2 transitions. To determine the effects of overexpressed HOXB7 on the response to IR exposure, several tests were done. Clonogenic survival assays were done using stably transfected MCF10A cells (FIG. 7B). Upon exposure to low-dose gamma radiation, MCF10A-Fl-HOXB7 cells had an ˜2-fold enhanced survival advantage over the vector-transfected and parental MCF10A cells (FIG. 8A). Similar results were obtained with SKBR3 cells (FIG. 8B) stably expressing HOXB7-YFP as shown by immunoblotting (data not shown).

G1-type (FIG. 8C) and G2-type (FIG. 8D) chromosomal aberrations in metaphase spreads were also examined from subcultured SKBR3 cells at various time points postirradiation (15). Cells in plateau phase were irradiated with 3 Gy, subcultured and examined for G1-type aberrations at metaphase. The frequency of aberrations was calculated in parental SKBR3, SKBR3-HOXB7-YFP, and SKBR3-vec cells and was significantly lower in SKBR3-HOXB7-YFP cells than in the other two groups (Student's t test, P<0.05). For G2-type aberrations, cells in exponential phase growth were irradiated with 1 Gy gamma radiation. Metaphases were examined for chromatid breaks and gaps. Fifty metaphases were scored for each postirradiation time point. Results for SKBR3-HOXB7-YFP-expressing cells were compared with those of vector-transfected (SKBR3-YFP) and parental (SKBR3) controls. SKBR3-HOXB7-YFP cells showed a significant decrease in G2-type chromosome aberrations as compared with parental control cells (Student's t test, P<0.035). These cells possessed an intact G2-M checkpoint (data not shown), although their elevated mitotic index (FIG. 8E) seems to indicate enhanced recovery and repair of DNA damage. The nature of the protection against radiation conferred by HOXB7 in these assays suggests that HOXB7 may affect DNA repair kinetics through the NHEJ pathway.

To explore this further, the DNA repair activities of HOXB7-containing nuclear extracts were tested in vitro by plasmid end-joining assays (14). This analysis revealed that expression of HOXB7-YFP in SKBR3 cells stimulated the end-joining activity almost 2.5-fold (FIG. 8F). These results were verified by knockdown of endogenous HOXB7 expression in breast cancer cells, MDA-MB-468, using antisense constructs. Transient transfection of HOXB7 antisense plasmids into MDA-MB-468 cells could suppress the expression of HOXB7 (>75%), and reduce NHEJ activity by ˜1.6-fold (data not shown). These results suggest a role for HOXB7 in stimulating DNA repair, and raise the possibility that it occurs through NHEJ.

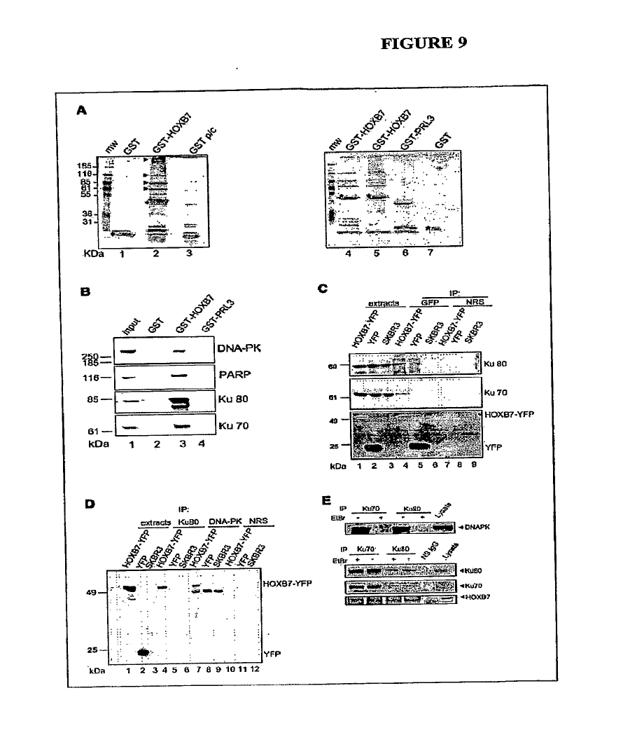

HOXB7 interacts with DNA repair proteins. To investigate whether HOXB7 plays a role in NHEJ, we attempted to identify proteins interacting with HOXB7 in breast cells. Cell extracts of SKBR3 (FIG. 9A, lanes 2 and 4) and MCF10A (FIG. 9A, lane 5) were fractionated by affinity chromatography on GST-HOXB7-Sepharose. Analysis of the proteins in column eluates by silver and Coomassie staining after SDS-PAGE revealed the presence of four polypeptides of approximate sizes of 70, 85, 110, and >250 kDa, which did not bind to the GST (lanes 1 and 7), or to the unrelated GST-fusion protein, GST-PRL3 (lane 6). Similar results were obtained with extracts of HMECs (MCF-12A) and breast cancer cells (MDA-MB-231; data not shown).

To identify the eluted proteins, several methods were used. Direct sequencing from PVDF membranes yielded results for the 85 kDa band, which identified Ku80 from an NH2-terminal 17-amino acid sequence (VRSGNKAAVVLCMDVGF (SEQ ID NO: 17)). For the 110 and 70 kDa protein bands, peptide mass fingerprints were obtained by MALDI-TOF and compared against those in public databases. Both ProFound and MS-FIT public database searches for the peptide mass maps obtained from the 110 and 70 kDa protein bands identified PARP and Ku70, respectively. Protein identities were confirmed by immunoblotting with antibodies against PARP, Ku80, and Ku70 (FIG. 8B). Because Ku70/80 are known binding subunits of DNA-PK, the high molecular weight band appearing at the top margin of the gel (>250 kDa) was predicted and confirmed as the DNA-PKcs by immunoblot analysis (FIG. 9B). The finding of several components of the DNA-PK complex suggested that DNA repair observed in the experiments described above are most likely mediated by NHEJ. We therefore focused our efforts on understanding the interaction of HOXB7 with components of the NHEJ complex.

Next, to test these interactions in intact cells, the associations between HOXB7 and Ku70, Ku80, and DNA-PKcs were analyzed in vivo by coimmunoprecipitation. A HOXB7-YFP fusion construct was stably introduced into the HOXB7-null breast cancer cell line, SKBR3. Fluorescence microscopy confirmed that HOXB7-YFP localized solely to the nucleus (data not shown). Immunoprecipitation with GFP antibodies (which also recognize the YFP variant) showed that Ku70 and Ku80 associated with HOXB7 in vivo (FIG. 9C, lane 4). Complementary immunoprecipitation using Ku80 (FIG. 9D, lanes 4-6) or DNA-PKcs (FIG. 9D, lanes 7-9) antibodies confirmed the presence of HOXB7-YFP in their complexes (lanes 4 and 7) following transient transfection of this construct into SKBR3 cells. Identical results were obtained when Fl-HOXB7 (Fl-HOXB7 pcDNA3) was transiently expressed in SKBR3 cells (data not shown). Complex formation was not affected by DNA damage from UV or IR (data not shown). To rule out the fact that these interactions were just the consequence of overexpressed HOXB7 protein, coimmunoprecipitation analyses were done using protein extracts of breast cancer cells, MDA-MB-435, which express detectable levels of endogenous HOXB7. The results showed that the same interaction between HOXB7 and Ku70/Ku80 occurs under physiologic conditions (data not shown).

The common DNA-binding properties of these proteins raised the possibility that the interactions observed above were mediated through DNA rather than through direct protein-protein interactions. We tested this possibility using two methods. First, DNase I had no effect on the binding of HOXB7 to Ku70/80 in HOXB7-transfected SKBR3 cells (data not shown). This finding was also verified more stringently using extracts of MCF-7 cells which express endogenous HOXB7. As previously shown (20), treatment with an intercalating agent (ethidium bromide) effectively blocked the interaction between Ku70/80 and DNA-PK because this reaction was completely dependent on the presence of DNA (FIG. 9E, top). In contrast, depletion of DNA in the extracts using ethidium bromide did not reduce the interactions between endogenous HOXB7 and Ku70 or Ku80 (FIG. 9E, bottom). It was also found that there was no evidence for the interaction of HOXB7 with two other DNA-binding proteins, i.e., BRCA-1 and E2F1 (data not shown). These results suggest that the interaction between HOXB7 and Ku70 and Ku80 are, in all likelihood, specific and not mediated by DNA.

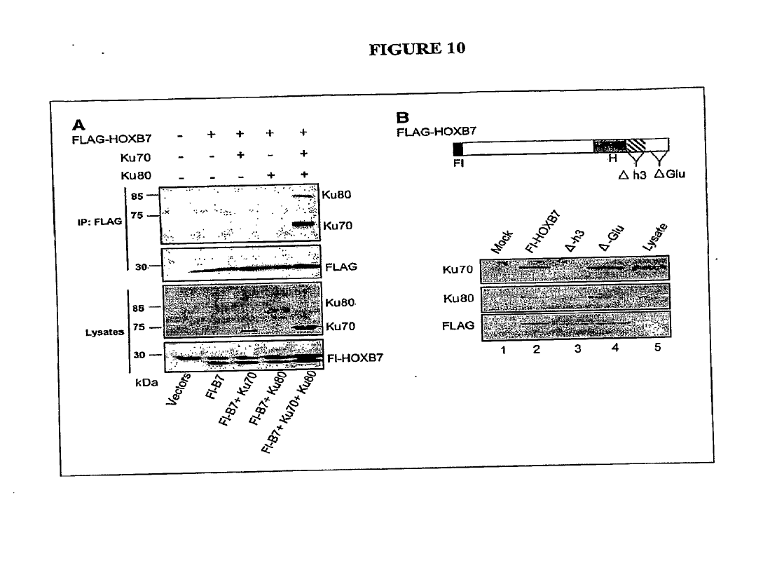

Because complexes formed by interactions between Ku70, Ku80, and DNA-PKcs were well-established, we investigated the nature of these complexes with HOXB7 and the order of their formation. Experiments introducing Fl-HOXB7 into CHO cells (FIG. 10A) showed that coexpression of human Ku70 and human Ku80 was required for the association of either Ku subunit with HOXB7. These results raise the possibility that Ku70/Ku80 heterodimer formation is a prerequisite for HOXB7 binding.

To define the region of HOXB7 that interacts with Ku70/80 proteins, full-length Fl-HOXB7 and HOXB7 with deletions of helix 3 of the homeodomain (HOXB7-Δh3) or of the glutamic acid tail (HOXB7-ΔGlu; FIG. 10B, top; ref. 11) were transfected into SKBR3 cells, and cell lysates were subjected to coimmunoprecipitation with FLAG antibody (FIG. 10B, bottom). The results showed that deletion of helix 3 from the homeodomain in HOXB7 (lane 3) completely abolished the interaction between HOXB7 and Ku70/80 proteins. In contrast, removal of the glutamic acid tail from HOXB7 (lane 4) did not affect the interaction. These results show that the integrity of the homeodomain is essential for the interaction between HOXB7 and Ku70/Ku80.

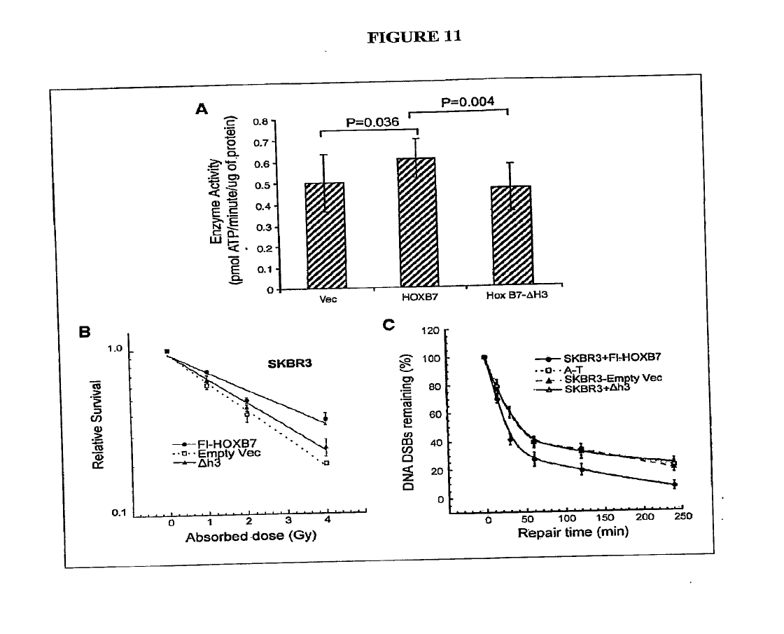

Expression of HOXB7 stimulates DNA-PK activity and enhances NHEJ. Because Ku70/80 is the DNA-binding subunit of DNA-PK, it is plausible that the observed interaction between Ku70/80 and HOXB7 may affect the catalytic activity of DNA-PK, and therefore NHEJ. To investigate the effect of HOXB7 expression on DNA-PK activity, MDA-MB-435 cells were transiently transfected with HOXB7 constructs (protein expression shown in data not shown). As shown in FIG. 11A, the expression of HOXB7 resulted in an increase in DNA-PK activity (P=0.036). Expression of HOXB7 lacking helix 3 of the homeodomain eliminated this effect, consistent with the finding that interaction between HOXB7 and Ku70/80 proteins is abolished by deletion of helix 3 from the homeodomain in HOXB7 (FIG. 10).

Because increased DNA-PK activity was abrogated by the deletion of helix 3 of HOXB7 (FIG. 11A), we further studied the effects of this deletion in clonogenic assays and in a DNA DSB repair assay. SKBR3 cells were transiently transfected with Fl-HOXB7, HOXB7-Δh3, or empty vector (data not shown) and exposed to IR; survival of the cell clones was compared with mock-irradiated (0 Gy) cells. Unlike full-length HOXB7 protein, HOXB7-Δh3 was unable to efficiently protect cells from the effects of IR. The difference in cell survival postirradiation between cells with full-length and those with mutant HOXB7 was significant (Student's t test, P<0.05; FIG. 11B). Thus, deletion of the h3 domain of HOXB7 eliminated protection against IR afforded by the full-length HOXB7 protein.

To determine if improved survival after radiation was a reflection of higher efficiency of repair of the DNA DSBs caused by the presence of HOXB7, SKBR3 cells transfected with Fl-HOXB7, HOXB7-Δh3, or empty vector were used. An ataxia telangiectasia cell line, GM5823, was used as a known repair-deficient control. Cells were irradiated with 50 Gy and lysed at different intervals after irradiation. Unrepaired DNA breaks were resolved by PFGE under nondenaturing conditions. SKBR3 cells were as inefficient at DSB repair as the ataxia telangiectasia cells. Cells overexpressing HOXB7 had the least amount of residual DNA DSBs. The effect of wild-type HOXB7 on residual DNA damage in cells was significant (Student's t test, P<0.05). Deletion of the h3 domain of HOXB7 abrogated the protective effect (FIG. 11C). Collectively, these experiments provide evidence that HOXB7 plays an role in DNA DSB repair. Furthermore, the h3 domain of HOXB7 is essential for the enhancement of DNA DSB repair through NHEJ.

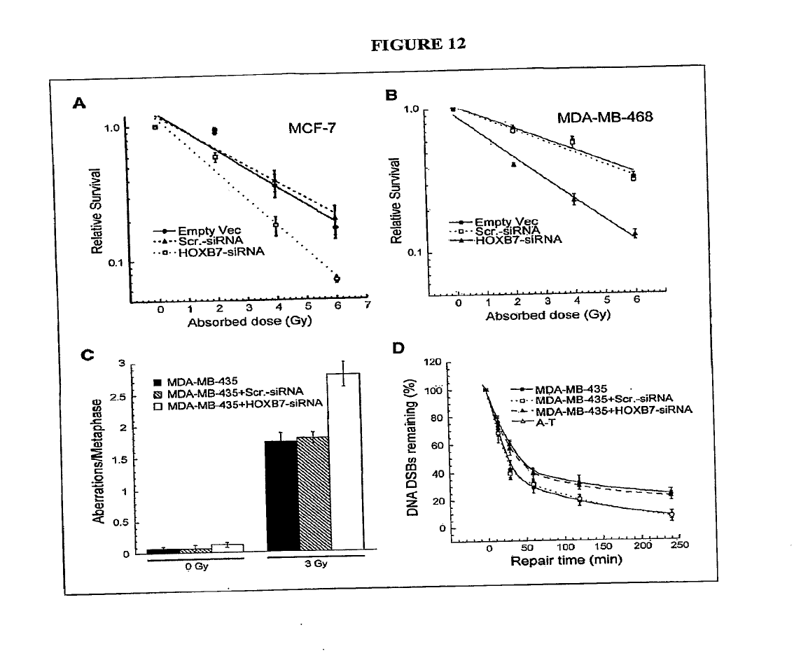

Knockdown of endogenous HOXB7 reduces the efficiency of DNA repair. Our results provide strong support that HOXB7 associates with members of the DNA-PK holoenzyme. Initial findings had pointed to enhanced DNA repair capability in HOXB7-overexpressing cells (8 and 11). To further test the relevance of these findings and the contribution of HOXB7 to DNA repair, survival after IR exposure following suppression of HOXB7 expression using siRNA was investigated. The expression of transfected HOXB7-specific siRNA into both MCF-7 (data not shown) and MDA-MB-468 cells (data not shown) reduced clonogenic survival significantly (P<0.01; FIGS. 12A and B). Next, chromosomal aberrations were analyzed at metaphase after irradiation of MDA-MB-435 cells with or without reduced levels of HOXB7 (data not shown). All categories of asymmetric chromosome aberrations were scored. The frequency of chromosomal aberrations was higher in cells with reduced levels of HOXB7, indicating defective repair of chromosome damage (FIG. 12C). Cells with HOXB7 knockdown showed significant differences (P<0.01) in chromosomal aberration frequencies compared with control cells (FIG. 12C). To further investigate the capacity of the G1-arrested cells to repair DSBs induced by IR, and to determine if this effect was mediated by HOXB7, PFGE was done on DNA from gamma-irradiated MDA-MB-435 cells transfected with scrambled siRNA or with HOXB7-specific siRNA (data not shown). Indeed, the specific siRNA treatment significantly (P<0.04) increased the level of unrepaired DNA DSB (FIG. 12D). Collectively, these data strongly suggest that HOXB7 could protect cells against DNA damage induced by IR exposure, possibly by conferring a higher efficiency of DNA DSB repair.

This study reports that HOXB7 is capable of transforming HMECs and of conferring resistance to IR. Resistance to IR seems to be through the binding of HOXB7 to proteins involved in DNA DSB repair, i.e., Ku70, Ku80, and DNA-PKcs. This is the first report demonstrating that HOXB7 acts as an oncogene, interacts with members of the DNA-PK holoenzyme, and plays a role in DNA DSB repair.

It is intriguing that HOXB7 is not only a transcriptional regulator, but also functions in DNA DSB repair. It was shown that one possible mechanism is by direct or indirect enhancement of the activity of a key enzyme, DNA-PK. Our results do not, at the present time, rule out transcriptional regulation of DNA repair genes as a possible mechanism. There is precedence for this premise. For example, when the Pem homeodomain-containing gene was expressed in murine Sertoli cells, it increased the number of DNA single-strand and double-strand breaks in the neighboring cells by regulating the expression of genes which affect DNA repair or chromatin remodeling (21).

Recent studies suggest that several other homeodomain-containing proteins may also play roles outside of transcriptional regulation, or have homeodomain-independent functions. Thus, the human proline-rich homeodomain protein, PRH (known as Hex in hematopoietic studies), interacts with eIF4E and inhibits its mRNA nuclear-cytoplasmic transport function (3). In addition, a variant of the CSX1 (CSX1b) protein lacking the homeodomain, retained its function (22), and a splice variant of Meis2 (Meis2e) lacking a complete homeodomain possessed some regulatory function (23). Studies in Drosophila have shown that the fushi tarazu protein has homeodomain-independent functions (24). Thus, novel functions of homeobox proteins, and those independent of their homeodomains, are beginning to be described.

It was shown that cell survival following IR was enhanced in four different HOXB7-expressing breast cancer cell lines. Our data indicated enhanced end-joined product formation and enhanced DSB repair (8, 11, and 12). When chromosomal damage and cell survival following IR was measured, it was found that somewhat less residual damage was apparent in cells expressing HOXB7, an effect that was reversed by HOXB7 silencing (FIG. 12A-D). These results indicate that cells expressing HOXB7 have enhanced survival and DNA repair rates compared with nonexpressing controls. The idea that a protein enhancing DNA repair can be an oncogene is somewhat counterintuitive. However, the NHEJ pathway for DNA DSB repair is error-prone compared with that of HR (25). Perhaps HOXB7-expressing cells, which have better survival post-IR exposure and have enhanced NHEJ activity, may harbor more potentially deleterious mutations, leading to a decrease in genomic stability. Enhanced resistance to IR could allow them to accumulate further mutations that initiate tumorigenesis.

Interactions similar to the ones reported in this study with Ku were also shown for Werner's syndrome protein (26). In addition, Werner's syndrome protein binds to many other proteins involved in DNA replication and repair, including Rad 52 (27), which we have also found to be associated with HOXB7 immunocomplexes.6 It is plausible that many other DNA repair-associated proteins form complexes with Ku and PARP, and that this type of complex formation may represent a hallmark of a subset of proteins involved in the same pathway regulating genomic stability. The evidence shown here, indicating roles for HOXB7 in enhanced cell survival and DNA repair rates after irradiation, suggests that HOXB7 joins other proteins in its involvement in DNA repair and maintenance of genomic stability. Taken together, it seems that HOXB7 may play a novel role in DNA repair by forming complexes with the Ku proteins.

### REFERENCES

1. Garcia-Fernandez J. The genesis and evolution of homeobox gene clusters. Nat Rev Genet 2005; 6:881-92.
2. Chen H, Sukumar S. Role of homeobox genes in normal mammary gland development and breast tumorigenesis. J Mammary Gland Biol Neoplasia 2003; 8:159-75.
3. Topisirovic I, Culjkovic B, Cohen N, Perez J M, Skrabanek L, Borden K L. The proline-rich homeodomain protein, PRH, is a tissue-specific inhibitor of eIF4E-dependent cyclin D1 mRNA transport and growth. EMBO J 2003; 22:689-703.
4. Richardson C, Horikoshi N, Pandita T K. The role of the DNA double-strand break response network in meiosis. DNA Repair (Amst) 2004; 3:1149-64.
5. Scott S P, Pandita T K. The cellular control of DNA double-strand breaks. J Cell Biochem 2006; 99:1463-75.
6. Karran P. DNA double strand break repair in mammalian cells. Curr Opin Genet Dev 2000; 10:144-50.
7. Schild-Poulter C, Pope L, Giffin W, et al. The binding of Ku antigen to homeodomain proteins promotes their phosphorylation by DNA-dependent protein kinase. J Biol Chem 2001; 276:16848-56.
8. Hochegger H, Dejsuphong D, Fukushima T, et al. Parp-1 protects homologous recombination from interference by Ku and Ligase IV in vertebrate cells. EMBO J 2006; 25:1305-14.
9. Schreiber V, Dantzer F, Ame J C, de Murcia G. Poly(ADP-ribose): novel functions for an old molecule. Nat Rev Mol Cell Biol 2006; 7:517-28.
10. Zhang X, Zhu T, Chen Y, Mertani H C, Lee K O, Lobie P E. Human growth hormone-regulated HOXA1 is a human mammary epithelial oncogene. J Biol Chem 2003; 278:7580-90.
11. Yaron Y, McAdara J K, Lynch M, Hughes E, Gasson J C. Identification of novel functional regions important for the activity of HOXB7 in mammalian cells J Immunol 2001; 166:5058-67.
12. Rubin E, Mittnacht S, VIIIa-Moruzzi E, Ludlow J W. Site-specific and temporally-regulated retinoblastoma protein dephosphorylation by protein phosphatase type 1. Oncogene 2001; 20:3776-85.
13. Shekhar M P, Werdell J, Tait L. Interaction with endothelial cells is a prerequisite for branching ductal-alveolar morphogenesis and hyperplasia of preneoplastic human breast epithelial cells: regulation by estrogen. Cancer Res 2000; 60:439-49.
14. Sharma G G, Gupta A, Wang H, et al. hTERT associates with human telomeres and enhances genomic stability and DNA repair. Oncogene 2003; 22:131-46.
15. Dhar S, Squire J A, Hande M P, Wellinger R J, Pandita T K. Inactivation of 14-3-3 influences telomere behavior and ionizing radiation-induced chromosomal instability. Mol Cell Biol 2000; 20:7764-72.
16. Pandita T K, Hittelman W N. Increased initial levels of chromosome damage and heterogeneous chromosome repair in ataxia telangiectasia heterozygote cells. Mutat Res 1994; 310:1-13.
17. Story M D, Mendoza E A, Meyn R E, Tofilon P J. Pulsed-field gel electrophoretic analysis of DNA double-strand breaks in mammalian cells using photostimulable storage phosphor imaging. Int J Radiat Biol 1994; 65:523-8.
18. Care A, Felicetti F, Meccia E, et al. HOXB7: a key factor for tumor-associated angiogenic switch. Cancer Res 2001; 61:6532-9.
19. Heppner G H, Miller F R, Shekhar P M. Nontransgenic models of breast cancer. Breast Cancer Res 2000; 2:331-4.
20. Yavuzer U, Smith G C, Bliss T, Werner D, Jackson S P. DNA end-independent activation of DNA-PK mediated via association with the DNA-binding protein C1D. Genes Dev 1998; 12:2188-99.
21. Wayne C M, Sutton K, Wilkinson M F. Expression of the pem homeobox gene in Sertoli cells increases the frequency of adjacent germ cells with deoxyribonucleic acid strand breaks. Endocrinology 2002; 143:4875-85.
22. Shiojima I, Komuro I, Mizuno T, et al. Molecular cloning and characterization of human cardiac homeobox gene CSX1. Circ Res 1996; 79:920-9.
23. Yang Y, Hwang C K, D'Souza U M, Lee S H, Junn E, Mouradian M M. Three-amino acid extension loop homeodomain proteins Meis2 and TGIF differentially regulate transcription. J Biol Chem 2000; 275:20734-41.
24. Hyduk D, Percival-Smith A. Genetic characterization of the homeodomain-independent activity of the Drosophila fushi tarazu gene product. Genetics 1996; 142:481-92.
25. Collis S J, DeWeese T L, Jeggo P A, Parker A R. The life and death of DNA-PK. Oncogene 2005; 24:949-61.
26. Li B, Navarro S, Kasahara N, Comai L. Identification and biochemical characterization of a Werner's syndrome protein complex with Ku70/80 and poly(ADP-ribose) polymerase-1. J Biol Chem 2004; 279:13659-67.
27. Baynton K, Otterlei M, Bjoras M, von Kobbe C, Bohr V A, Seeberg E. WRN interacts physically and functionally with the recombination mediator protein RAD52. J Biol Chem 2003; 278:36476-86.

### Example 3

### Experimental procedures

Cell Lines, Cell Culture and Reagents.

pcDNA3 vector or pcDNA3-Flag-HOXB7 were stably transfected into MCF-10A cells or MCF-7 cells by use of Effectene (Qiagen, Valencia, Calif.). MCF-7-LTED, the estrogen hypersensitive MCF-7 subline was generated from MCF-7 cells by long-term culture under estrogen-deprived conditions and are called long-term estradiol-deprived (LTED) cells^{18,19}, and were kindly gifted by Dr. Santen. LTED cells are refractory to tamoxifen but sensitive to fulvestrant (Martin La., 2005). MCF-7-TAMLT Long-term tamoxifen-stimulated tumor (MCF-7 TAMLT) extracts, kindly provided by V. Craig Jordan, were developed by re-transplanting growing estradiol-dependent MCF-7 tumors into new athymic mice and treating the mice with tamoxifen for more than 5 years^{20,22}. Fulvestrant and Iressa (gefitinib) were provided by Astrazeneca (Cheshire, U.K.).

Luciferase Reporter Assay.

Transient transfection was performed with the respective promoter-luciferase constructs. Results were normalized to the level of B-galactosidase activity in the samples. The EGFR promoter reporter plasmids were a kind gift of Dr. Alfred C. Johnson (Bethesda, Md.). ERE-tk-LUC was a generous gift from Dr. Elaine Alarid (Madison, Wis.).

### Small Interfering RNA Preparation and Transfection.

The siRNA sequences used for targeting human HOXB7: 5′-ATA TCC AGC CTC AAG TTC G-3′ (SEQ ID NO: 1) and 5′-ACT TCT TG TGC GTT TGC TT-3′ (SEQ ID NO: 2). The two HOXB7 siRNA expression plasmids were mixed 1:1 for transfection by use of Effectene (Qiagen).

Xenograft Analysis.

3×10⁶ cells of MCF-7-vec or MCF-7-B7 were suspended in 100 μl PBS/Matrigel (1:1) and injected s.c into the female 3- to 4-week-old BALB/c nu/nu athymic mice (Harlan, Sprague Dawley, Madison, Wis.), which simultaneously received a 60-day slow release pellet containing 0.72 mg of 17β-estradiol and/or 5 mg tamoxifen (Innovative Research of America, Southfield, Mich.). Fulvestrant was dissolved in 100% ethanol and diluted in sesame oil; 5 mg was injected s.c. twice a week²⁰. Animals were observed once a week. At necropsy, primary tumors, liver, lung and spleen were evaluated for the presence of macroscopic tumors. Tissue samples of the primary tumor and organs were fixed in 4% paraformaldehyde and stained with H&E to assess histomorphology.

Chromatin Immunoprecipitation (ChIP) Assay.

Formaldehyde for crosslinking DNA was directly added to the medium of 1×10⁶ MCF-7 cells transiently transfected with pcDNA3-Flag-HOXB7. Cells were harvested and sonicated to shear DNA to lengths between 300 and 600 bp. After centrifuging samples for 10 min at 13,000 rpm at 4° C., the supernatant was pre-cleared with 75 μl of salmon sperm DNA/protein A-agarose, (50% slurry) for 30 min at 4° C. with agitation. 2 μg of anti-Flag M2 antibody or control IgG was then added to the supernatant fraction for incubation overnight at 4° C. with rotation. Then 60 μl of salmon sperm DNA/protein A-agarose was added to collect the antibody-histone complex. The protein A-agarose-antibody-histone complex was extensively washed for 5 min, and heated at 65° C. for 4 h to reverse histone-DNA crosslinks. The DNA was recovered by phenol/chloroform extraction and ethanol precipitation. PCR primers for EGFR promoter region (sense 5′-ATT ATC CGA CGC TGG CTC TA-3′ (SEQ ID NO: 18), anti-sense 5′-CGG GTG CCC TGA GGA GTT AA-3′ (SEQ ID NO: 19); sense 5′-TTG GCT CGA CCT GGA CAT A G-3′ (SEQ ID NO: 20), anti-sense 5′-GAG GGA GGA GAA CCA GCA G-3′ (SEQ ID NO: 21)); PCR primers for HOXB7 promoter region (sense 5′-GCC CCT CTC GGA AAT TAA CTC-3′ (SEQ ID NO: 22), anti-sense 5′-AGG AGC AGA GGA GGA GGA GA-3′ (SEQ ID NO: 23)) The PCR program was set with an initial melting step at 94° C. for 3 min, then 35 cycles of (94° C. for 45 sec, 58° C. for 45 sec, and 72° C. for 50 sec). The PCR products were subsequently analyzed on agarose gel by electrophoresis.

Multiple factors including long term treatment with tamoxifen are involved in the development of selective estrogen receptor modulator resistance of ERα-positive breast cancer. Many underlying molecular events that confer resistance are known but a unifying theme is yet to be revealed. We provide evidence that HOXB7 overexpression renders MCF-7 cells resistant to tamoxifen via cross-talk between RTKs and ERα signaling. Tamoxifen treatment showed progressively increasing levels of HOXB7 expression over time, accompanied by concomitant increased expression of EGFR/HER2 and ERα. HOXB7 overexpression might be a one of the key events in the initiation and maintenance of tamoxifen resistance. Consistently, higher expression levels of HOXB7 significantly correlate with poorer disease free survival in ERα+ breast cancer patients. These studies suggest that HOXB7 could act as a master regulator orchestrating two major groups of target molecules in the oncogenic hierarchy. Functional antagonism of HOXB7 might be of great importance to circumvent tamoxifen resistance.

The selective estrogen-response modulator (SERM), tamoxifen, is the most commonly used adjuvant treatment for postmenopausal women with early-stage estrogen receptor-α (ERα)-positive breast cancer. Despite the relative safety and significant anti-neoplastic and chemopreventive activities of tamoxifen, many breast tumors either remain refractory or initially responsive to SERM develop resistance and ultimately recur. Tamoxifen resistance can be classified into two categories: intrinsic or acquired^{1,2}. Interestingly, ER expression is maintained at detectable levels in the majority of the tumors with acquired resistance. In these tumors, ER continues to regulate tumor proliferation^{3,4}. Two-thirds of patients who relapse on tamoxifen respond to the pure ER-antagonist, fulvestrant, or to aromatase inhibitors¹.

Homeobox genes are regulatory genes encoding nuclear proteins that act as transcription factors during normal development and HOXB7 expression^{5,6}. One of these, HOXB7, is involved in a variety of developmental processes, including hematopoietic HOXB7 expression and lymphoid and mammary gland development. The role of HOX genes in breast cancer development is largely unexplored. We have recently identified HOXB7 as one of few prominent genes, the expression of which were significantly elevated in both the primary cancer and distant metastasis by both micro-array and real-time PCR (Wu, 2006). In culture, HOXB7 transforms mammary epithelial cells, MCF10A, in vitro and promotes epithelial/mesenchymal transition and invasion in a variety of cell lines through activation of the RHO/RAC pathway¹⁰. It was also reported HOXB7 transfection promotes cell proliferation in SKBR3 breast cancer cells along with enhanced tumorigenicity and angiogenesis-7,8.

Presented herein is evidence that HOXB7 overexpression in breast epithelial cells confers tamoxifen resistance in ER-positive cells through increased expression of EGFR/HER2 and ERα and their respective signaling. By studying ER-positive, Tam-resistant cells, and MCF-7 cells over time as they acquired Tam-resistance, we show that elevation of HOXB7 expression might be one of the key steps in the acquisition and maintenance of anti-estrogen resistance in breast cancer. HOXB7 could serve as a master regulator in the transition of breast cancer cells to estrogen-independence, tamoxifen-resistance and acquisition of an aggressive phenotype, a hallmark of poor prognosis. Functional antagonism of HOXB7 might be of great importance to breast cancer therapeutics.

HOXB7 Expression Promotes Breast Tumorigenesis.

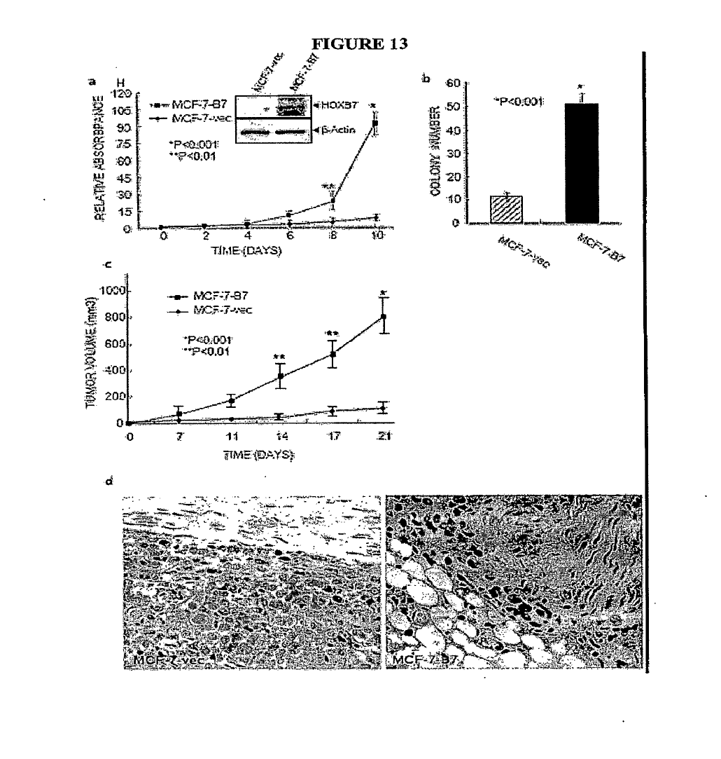

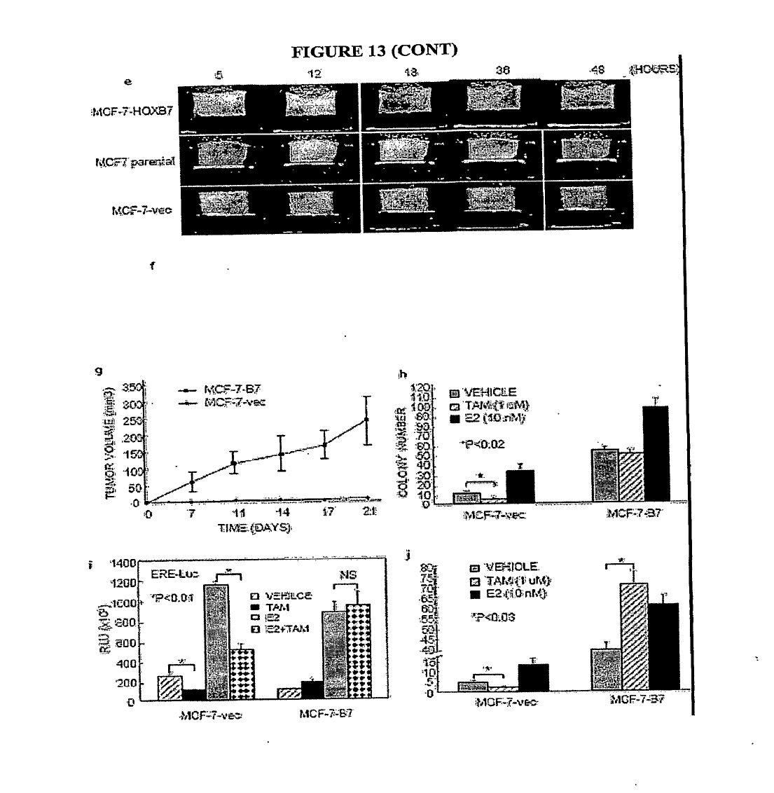

Breast cancer cells, MCF-7, are estrogen-dependent for growth in vitro and in vivo and are susceptible to the cytostatic/cytotoxic effects of tamoxifen. Stable expression of a HOXB7 expression vector in MCF-7 cells (pooled clones, designated MCF-7-B7) (FIG. 13a) enabled the cells to proliferate much faster than the vector control cells (designated MCF-7-vec) in monolayer cultures (FIG. 13a) and significantly enhanced colony formation (FIG. 13b). When transplanted to the athymic nude mice s.c. in presence of exogenous estrogen supplementation, MCF-7-B7 cells formed faster growing and larger tumors compared to the MCF-7-vec cells (FIG. 13c). Tumors formed by MCF-7-vec cells were grossly well-defined and loosely attached to surrounding tissue. MCF-7-B7 cells, on the other hand, grew as highly invasive tumors firmly attached to surrounding tissues, infiltrating the underlying skeletal muscle and fat tissue (FIG. 13d). Consistently, magnetic resonance imaging (MRI) analysis revealed that tumors forming by MCF-7-B7 cells are highly invasive in vitro (FIG. 13e) and are significantly hypervascular in vivo (FIG. 13f). These data showed that HOXB7 overexpression promotes invasive and aggressive growth of the MCF-7-B7 cells.

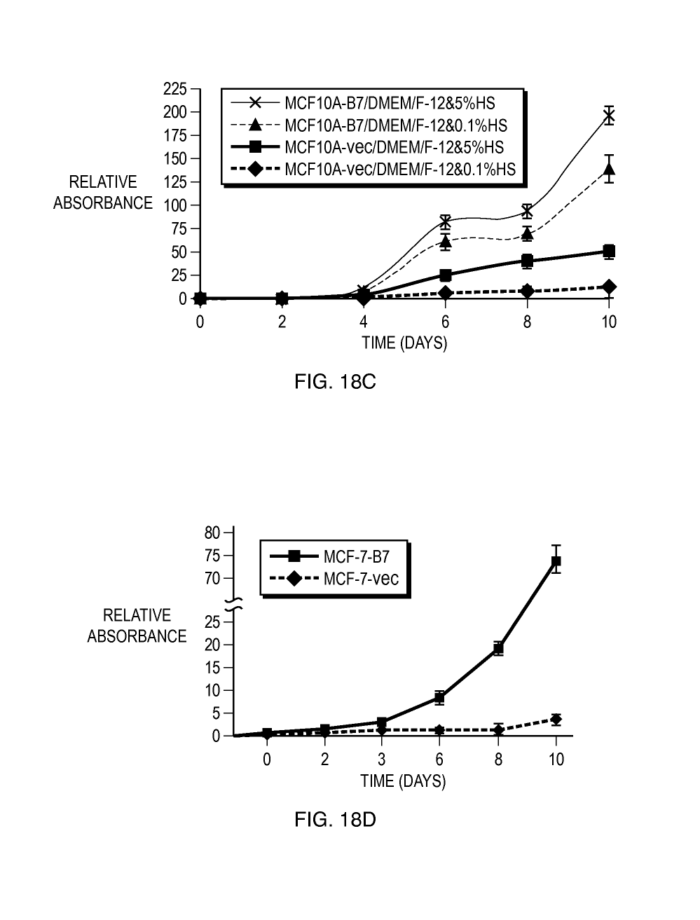

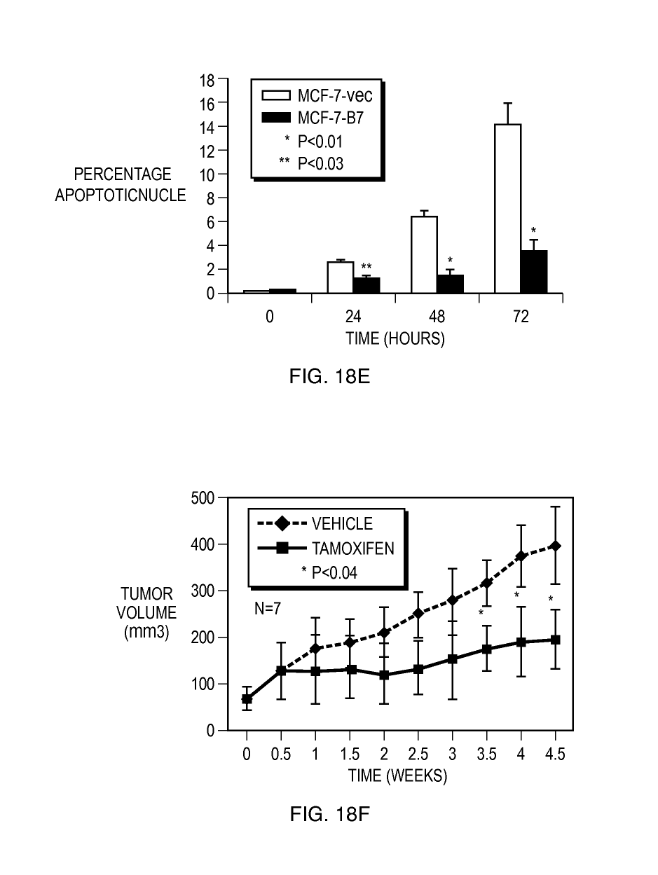

One of the hallmarks of cancer is self-sufficiency in growth signals (Weinberg, 2000). We demonstrated here that HOXB7 overexpressing in both MCF-10A and MCF-7 cells acquire much reduced dependence on environmental nutrient, as evidenced by the capacity of MCF-10A-B7 cells to grow in low growth-factor supplemented medium (FIG. 18) and MCF-7-B7 cells to grow in estrogen-deprived medium. But barely MCF-10A-vec cell or MCF-7-vec cells were able to grow under the conditions above (FIG. 13j, 18). In addition, MCF-7-B7 cells formed rapidly growing tumors in athymic nude mice even in the absence of exogenous estrogen supplementation (FIG. 13g). In contrast, the MCF-7-vec cells did not form palpable tumors in vivo in the absence of exogenous estrogen supplementation. Thus, HOXB7 overexpression enabled MCF-7 cells to largely circumvent the need for exogenous estrogen for growth.

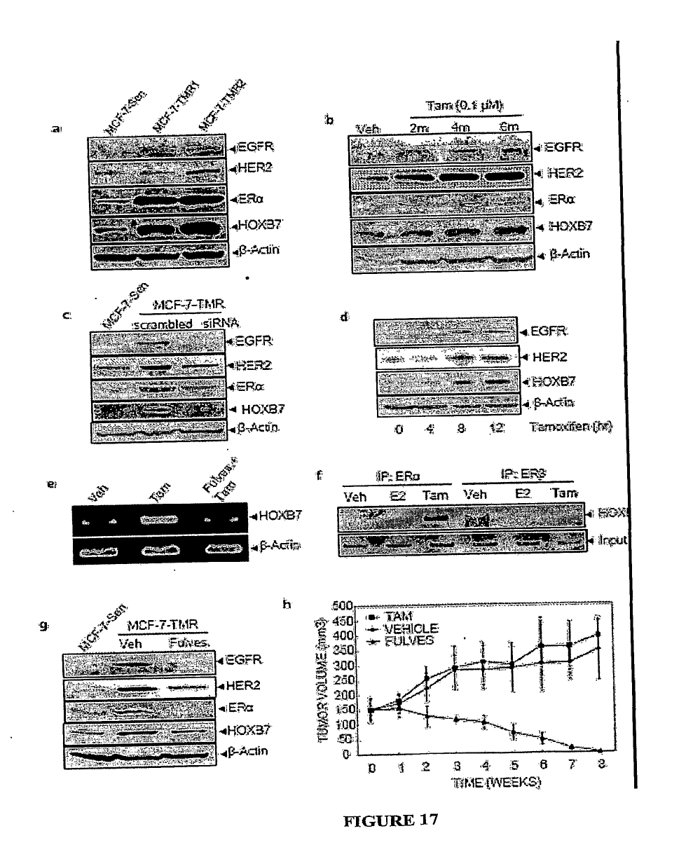

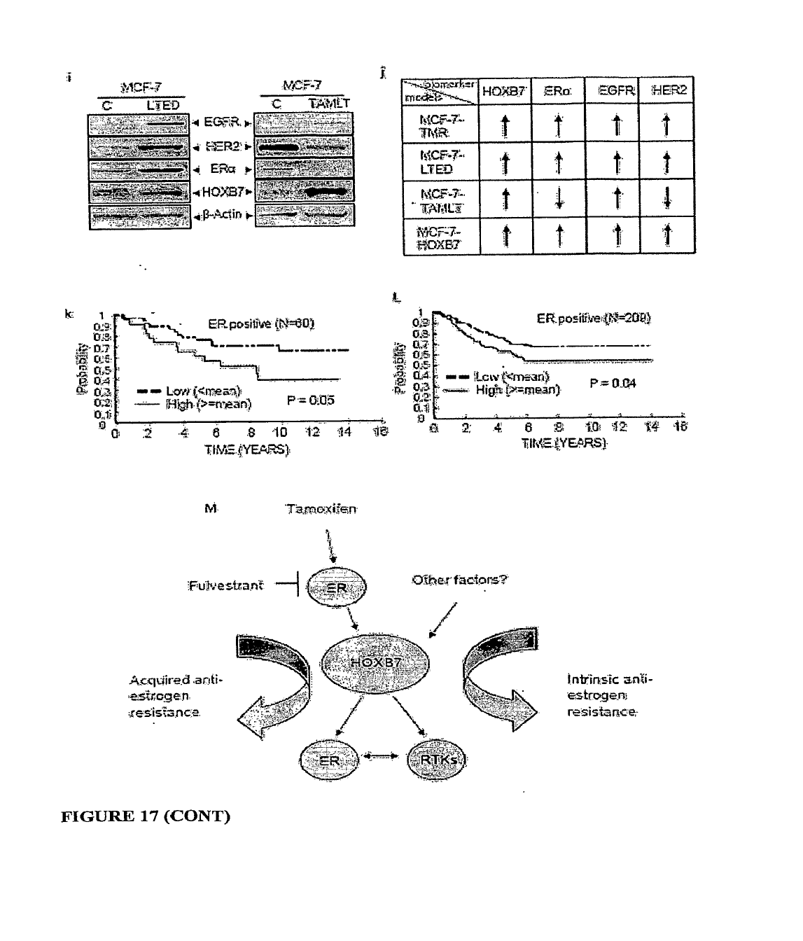

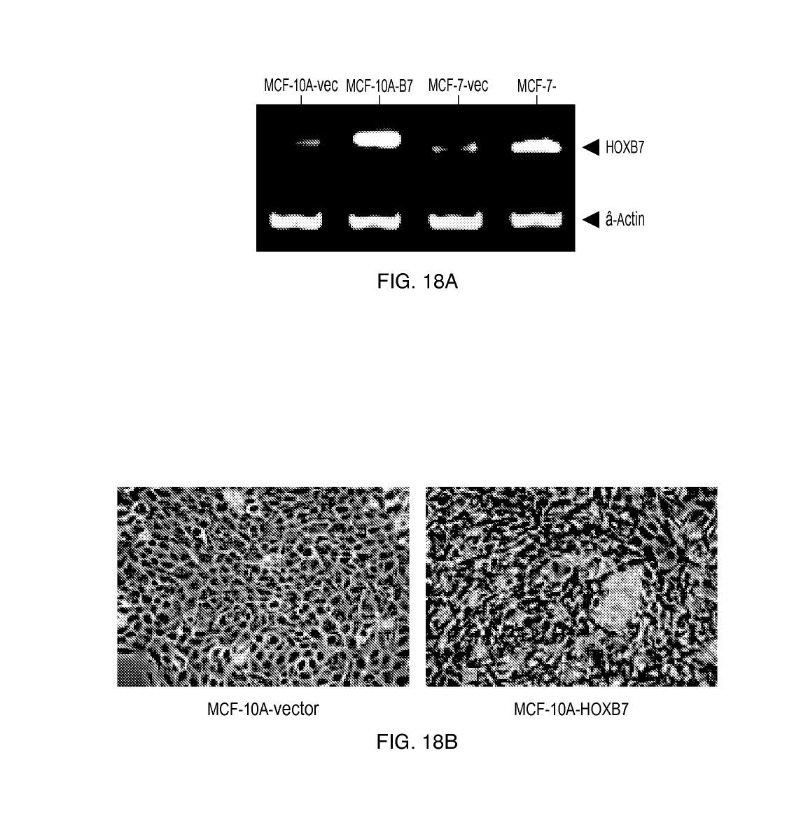

Reduced estrogen requirement of the ER-positive cells is often linked to their resistance to tamoxifen treatment^{11,12}. Consistent with those observations, MCF-7-B7 cells displayed loss of sensitivity to the inhibitory effects of tamoxifen in culture (FIG. 13h). The tamoxifen-resistant property of MCF-7-B7 cells was also verified by the inability of tamoxifen to attenuate estrogen-stimulated ERE-luc reporter activity in these cells (FIG. 13i). Further, MCF-7-B7 cells formed more colonies in soft agar under estrogen-deprived conditions than the cognate control cells, and its growth was even enhanced in the presence of tamoxifen, suggesting that tamoxifen might be converted from an antagonist to an agonist in these cells as a result of HOXB7 overexpression (FIG. 13j). In addition, tamoxifen treatment exhibited no inhibitory effect on established tumors of MCF-7-B7 cells in nude mice (FIG. 17h), unlike its effect on the xenografts of parental MCF-7 cells (FIG. 18). These results provided further lines of evidence, both in vitro and in vivo, that HOXB7 conferred not only estrogen-independence, but also tamoxifen-resistance to breast cancer cells.

Molecular Effectors of HOXB7 in Tamoxifen Resistance

Further effort was focused on the mechanism by which HOXB7 overexpressing cells acquire self-sufficiency in growth signals as suggested above. Receptor tyrosine kinases (RTKs) are major mediators of the signaling network that transmit extra-cellular signals into the cells, and control cellular HOXB7 expression and proliferation. We found that an array of RTKs reported to be frequently expressed in breast cancer¹³ were upregulated by HOXB7 in several transfected breast cancer cell lines (data not shown). Further analysis of two members of the ErbB/HER family of protein-tyrosine kinases was performed to shed light on the mechanism by which HOXB7 regulates these RTKs.

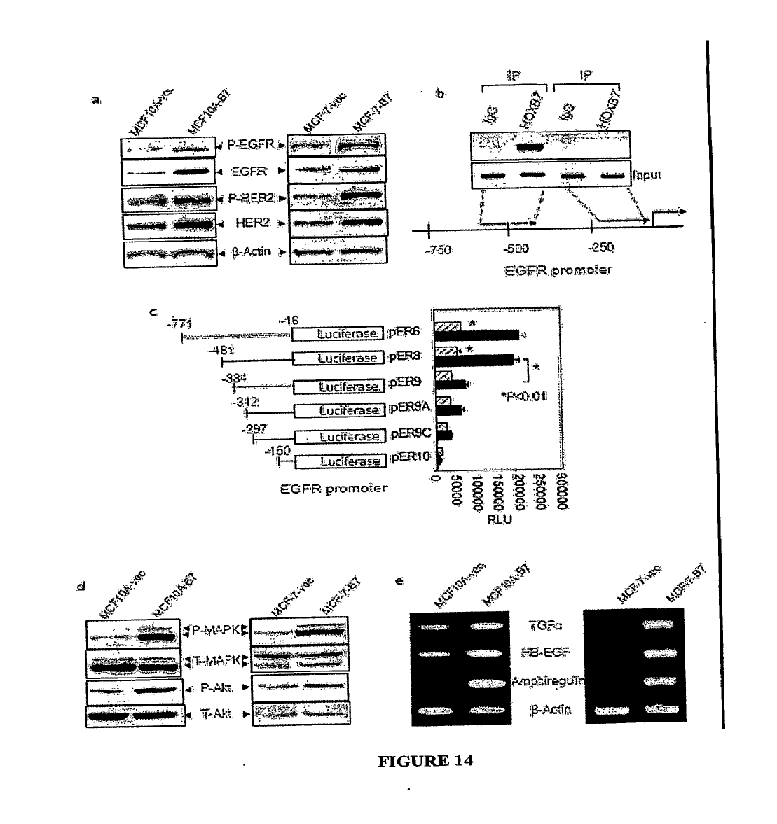

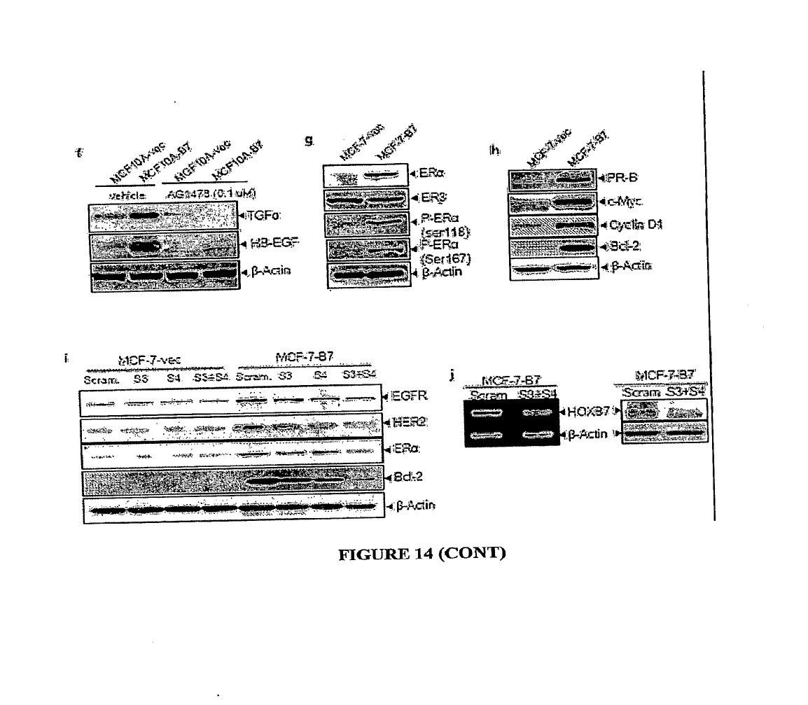

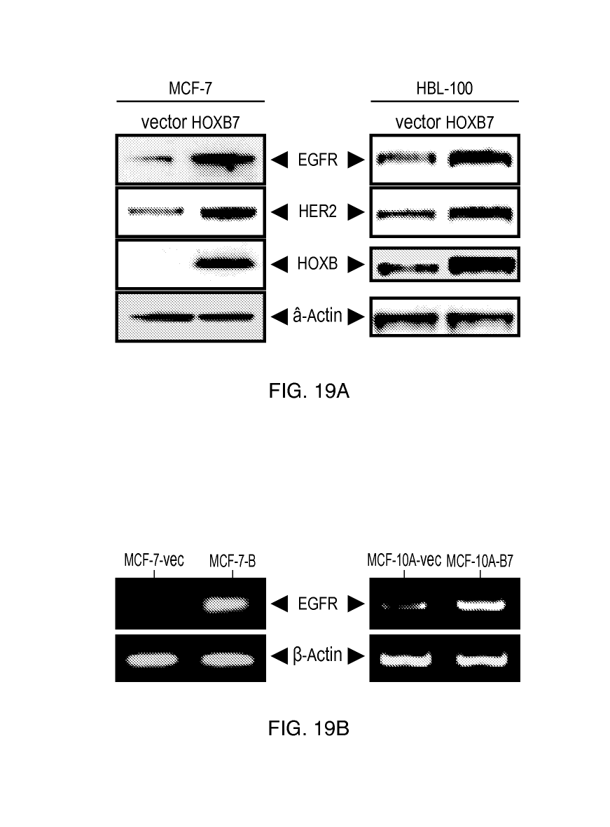

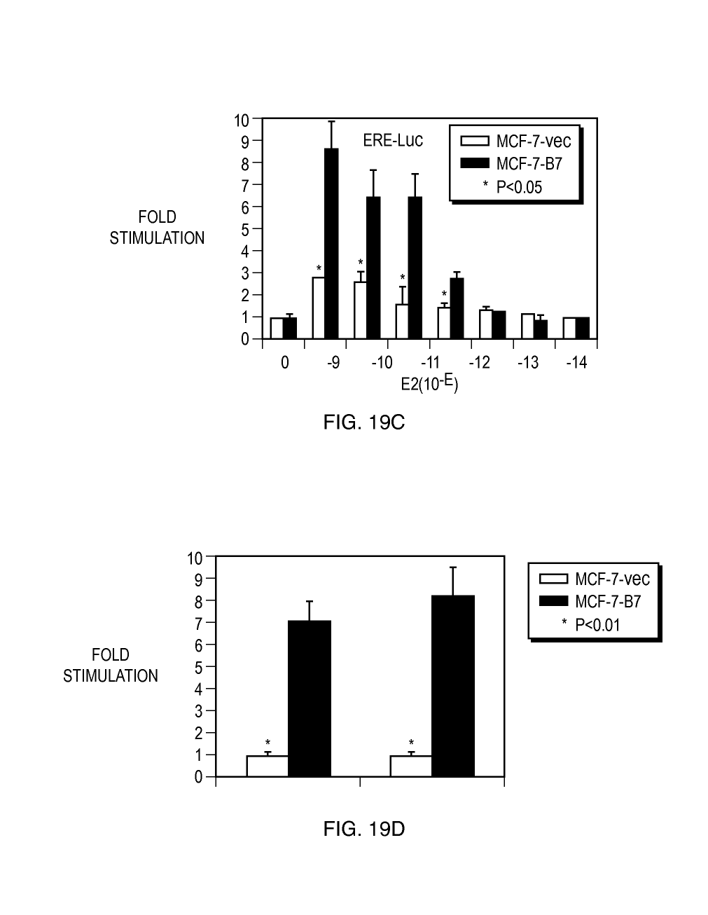

The ErbB/HER receptors including HER1/EGFR and HER2/Neu and their cognate ligands are involved in the pathogenesis of different types of carcinomas including breast cancer¹⁴. We found that HOXB7 stable overexpression caused an increased expression of EGFR and HER2 in both MCF-10A and MCF-7 cells (FIG. 14a). Transient expression of HOXB7 in MCF-7 and human mammary epithelial cells, HBL-100, also resulted in higher levels of EGFR and HER2 (FIG. 19). To explore the mechanism by which HOXB7 regulates EGFR expression, we performed semi-quantitative RT-PCR analysis. The results showed an increased expression of EGFR at the mRNA level in both MCF-10A and MCF-7 cells overexpressing HOXB7 (FIG. 19), suggesting that the EGFR promoter might be transcriptionally regulated by HOXB7. To test this premise, CHIP assays were performed to determine if HOXB7 binds directly to the EGFR promoter. A single putative HOXB7-binding site was identified in the 800 by EGFR promoter (FIG. 14b). Luciferase reporter constructs containing serial deletions of the EGFR promoter¹⁵ were co-transfected into MCF-7 and MCF-10A cells along with the HOXB7 expression plasmids. As shown in FIG. 14c, both pER6-luc containing regions −771 to −16, and pER8-luc containing regions −484 to −16 were activated by 2.5- to 3-fold by HOXB7, whereas pER9-luc containing nucleotides −389 to −16, the −292 to −16, or the −150 to −16 regions were activated at much lower levels in MCF-7 cells. These results were consistent with the CHIP assay data. Similar results were obtained in MCF-10A cells (data not shown).

Elevated tyrosine phosphorylation at the kinase domain was observed in MCF-10A-B7 and MCF-7-B7 cells (FIG. 14a). Consistent with previous observations¹⁴, two of the major downstream pathways, p44/42 MAPK and PI3K/Akt were activated in HOXB7 expressing cells (FIG. 14d). Elevated activation of EGFR/HER2 as a result of HOXB7 overexpression prompted us to examine a possible over-production of known ErbB/HER ligands. Indeed, autocrine/paracrine production of multiple ErbB/HER ligands (Amphiregulin, TGFα and HB-EGF)¹⁴ was observed in MCF-10A-B7 and MCF-7-B7 cells (FIG. 14e). Consistent with the increased mRNA levels, a significant increase of TGFα and HB-EGF expression was detected at the protein level in MCF-10A-B7 cells (FIG. 140. The elevated expression of TGFα and HB-EGF was significantly abrogated by the pharmacological inhibition of EGFR activity using the EGFR kinase inhibitor AG1478, suggesting the possible existence of a positive feedback mechanism for the synergistic activation of EGFR/HER2 pathways as a result of HOXB7 expression in MCF10A cells. However, such a possible feedback via EGFR was not observed in MCF-7-B7 cells (data not shown). It is possible that in MCF-7-B7 cells, an alternative pathway regulates the overproduction of autocrine/paracine ErbB/HER ligands mediated by HOXB7 overexpression (see below).

Since tamoxifen sensitivity is often related to ER function, we investigated whether this is the case in MCF-7-B7 cells. Elevated expression of ERα was detected in HOXB7-overexpressing cells (FIG. 14g). Concomitant increases in the expression of four estrogen-responsive genes (PR, Bcl-2, Cyclin D1, and c-Myc) suggested enhanced ER function in MCF-7-B7 cells (FIG. 14h). These findings provided further evidence that HOXB7 overexpression simultaneously targeted both non-genomic/ligand-independent^{1,16,17} (FIG. 14g, 14h) and genomic/ligand-dependent signaling of ERα in MCF-7 cells as indicated by its hyper-sensitivity to lower doses of estrogen stimulation (FIG. 19).

To verify the observed HOXB7-dependent alterations of gene expression, HOXB7 specific siRNAs (designated S3, S4) (Wu X, 2007) were applied. Knocking down HOXB7 levels in MCF-7-B7 cells was sufficient to reverse the upregulated expression of ERα, EGFR, HER2 as well as Bcl-2, the downstream target gene (FIG. 14i, 14j). A second, independently derived MCF-7-B7 cell line consisting of pooled clones recapitulated all the molecular changes presented here (data not shown). Thus, it appears that EGFR/HER2 and ERα are molecular effectors of the overexpressed HOXB7 in MCF-7 cells. Elevated ERα expression observed as a consequence of HOXB7 overexpression in MCF-7 cells could be indirect or conditioned by other variables in the system (data not shown). Collectively, we have provided evidence demonstrating overexpression of receptors and ligands, activation of downstream effectors, direct binding of HOXB7 to EGFR promoter and reversal of these effects by siRNA to HOXB7.

HOXB7 Overexpression Converts Tamoxifen into an Agonist

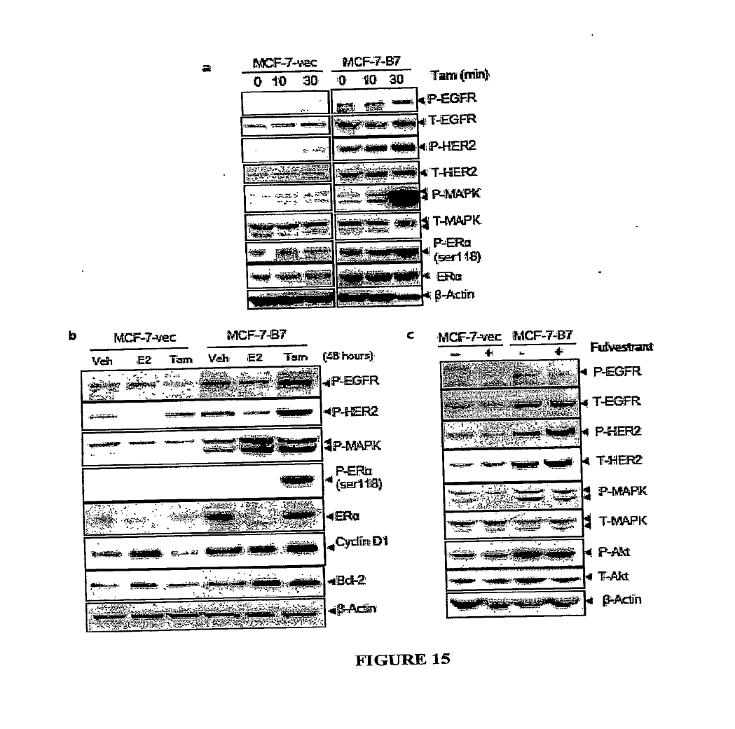

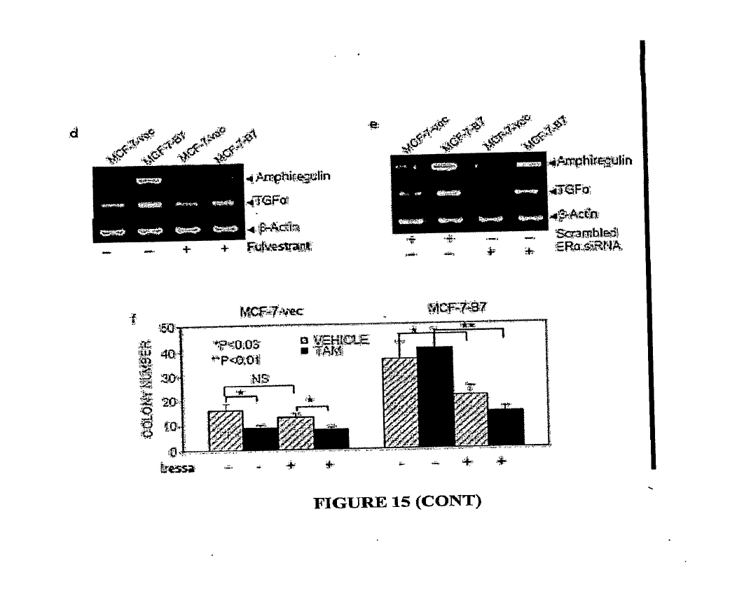

Since the data on in vitro and in vivo proliferation assays suggested that tamoxifen might be converted from an antagonist into an agonist upon HOXB7 overexpression in MCF-7 cells, we sought evidence for this change at the molecular level. Estrogen deprived MCF-7-vec and MCF-7-B7 cells were treated with 1 μM tamoxifen for a short period as indicated (FIG. 15a). Tamoxifen treatment of MCF-7-B7 cells led to elevated levels of active forms of EGFR and HER2, indicating tamoxifen elicited ER dependent pathway exert a rapid and direct crosstalk with EGFR/HER2 dependent pathways as a result of HOXB7 overexpression. Consistently, tamoxifen treatment potently stimulates the activation of p44 MAPK and ER phosphorylation at Ser 118 site in MCF-7-B7 cells but not in MCF-7-vec cells (FIG. 15a). We further examined a panel of estrogen-responsive proteins and the activation status of EGFR and HER2 in response to tamoxifen treatment over an extended period. Tamoxifen treatment of MCF-7-B7 cells in estrogen deprived medium for 48 hours led to the expression of a significantly higher level of active forms of EGFR and HER2, whereas tamoxifen treatment of MCF-7-vec cells resulted in either a reduction in levels of the active form of EGFR or about 1.5-fold increase of active form of HER2 (FIG. 15b). Consistent with an increase in EGFR/HER2 activity in MCF-7-B7 cells, tamoxifen treatment results in an also increased p44/42 MAPK activity and potent phosphorylation of ERα at serine 118. Further, in contrast to its effects in MCF-7-vec cells, tamoxifen showed a potent estrogen-like agonistic effect on the expression of Cyclin D1 and Bcl-2. Since HOXB7 overexpression caused a more than 20-fold increase of Bcl-2 expression in MCF-7-B7 cells, a much shorter time exposure of the film was shown the lane 4-6 compared to that of the lane 1-3 in the figure (FIG. 15b). Thus, in the setting of HOXB7 overexpression, tamoxifen appears to behave as an agonist to activate EGFR/HER2 and their downstream p44/42 MAPK pathway, with resultant increased expression of estrogen-dependent genes.

To further examine possible cross-talk of EGFR/HER2 and ER signaling as a result of HOXB7 overexpression, the EGFR-specific inhibitor, Gefitinib, and the pure ER antagonist, fulvestrant (ICI 182780, Faslodex), were utilized. Fulvestrant treatment reduced HOXB7 overexpression-related increases in EGFR and p44/42 MAPK activity, but not HER2 and Akt activity (FIG. 15c). Consistent with reduced EGFR activity, a significant reduction in levels of the autocrine/paracrine EGFR ligands (Amphiregulin and TGFα) was observed in fulvestrant-treated MCF-7-B7 cells (FIG. 15d). The specificity of effects of fulvestrant was also verified by the use of ERα-specific siRNA transfection (FIG. 15e). Thus, ER dependent pathway might be responsible for elevated levels of the autocrine/paracrine EGFR ligands in MCF-7-B7 cells, which may result in elevated EGFR/HER2 signaling. Consistent with our predictions, abrogation of EGFR activity in MCF-7-B7 cells by gefitinib significantly re-sensitized the cells to tamoxifen treatment, whereas vector control cells displayed minimal response to tamoxifen (FIG. 15f). Thus, these results suggest that cross-talk between ER and EGFR signaling in MCF-7 cells upon HOXB7 overexpression could result in tamoxifen resistance.

### HOXB7 as a Potential Therapeutic Target

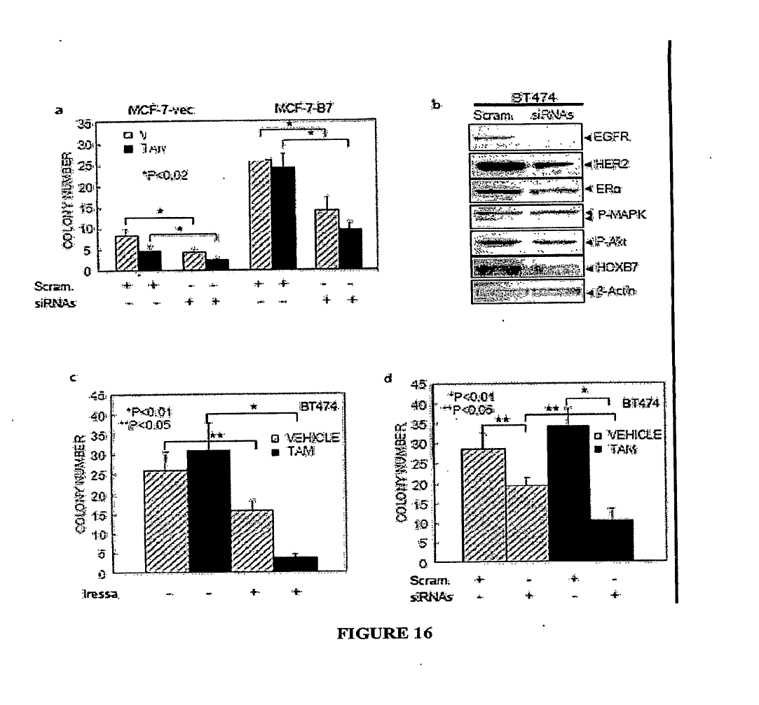

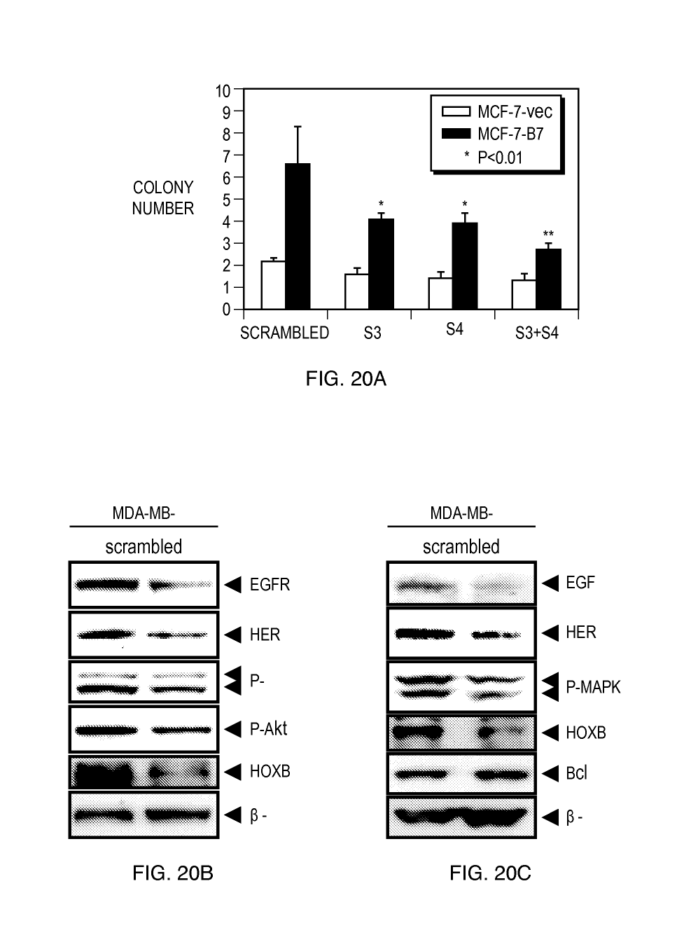

Thus far, our data has provided evidence to support the notion that HOXB7 overexpression in MCF-7 cells impacts on EGFR/HER2 and ER expression and their respective signaling. These findings suggest that HOXB7 action lies upstream of these pathways. We therefore investigated whether abrogation of HOXB7 expression is sufficient to reverse the much-enhanced malignant traits in MCF-7-B7 cells. We have shown that abrogation of HOXB7 expression by HOXB7-specific siRNAs significantly reduced increased HOXB7-afforded expression of EGFR, HER2, as well as ERα, Bcl-2 (FIG. 14i). Consistent with this finding, HOXB7-specific siRNAs significantly reduced increased colony formation in soft agar and further re-sensitized MCF-7-B7 to tamoxifen treatment (FIG. 16a). In line with these findings, transfection of HOXB7 siRNAs to parental MCF-7 cells was also able to cause a reduction in colony formation and enhanced sensitivity to tamoxifen treatment, although to a lesser extent (data not shown). p44/42 MAPK and Akt activity was also reduced by HOXB7 siRNA expression in breast cancer cell lines, both ER positive-MCF-7 and T47D (data not shown), and ER-negative-MDA-MB-435 and MDA-MB-468 (FIG. 20). Of note, abrogation of HOXB7 expression in MDA-MB-435 and MDA-MB-468 cells was sufficient to reduce the endogenous level of EGFR and HER2 (FIG. 20), suggesting that HOXB7 might be an attractive anti-cancer target in ER-negative tumors. Direct evidence for a role for HOXB7 was sought in an un-manipulated, tamoxifen-resistant breast cancer cell line, BT474, in which both HER2 and HOXB7 are amplified and overexpressed⁹. Here, reduction of endogenous HOXB7 expression using HOXB7-siRNAs was sufficient to reduce the expression levels of HER2, EGFR and ERα, with regained sensitivity to tamoxifen (FIG. 16b, 16d). Abrogation of EGFR/HER2 dependent pathway apparently is important for HOXB7-siRNAs elicited effect since EGFR/HER2 specific inhibitor Iressa dramatically converted tamoxifen from a partial agonist to a potent antagonist in BT474 cells (FIG. 16c). Thus, HOXB7 might be a drug target, whose functional antagonism impinges on multiple pathways important to tamoxifen resistance.

### HOXB7 in the Acquisition of Anti-Estrogen Resistance

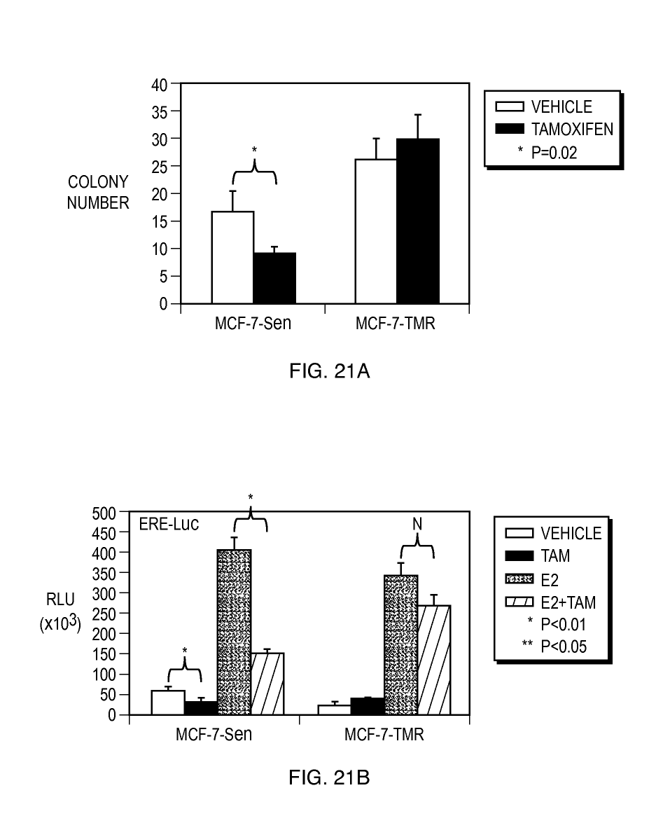

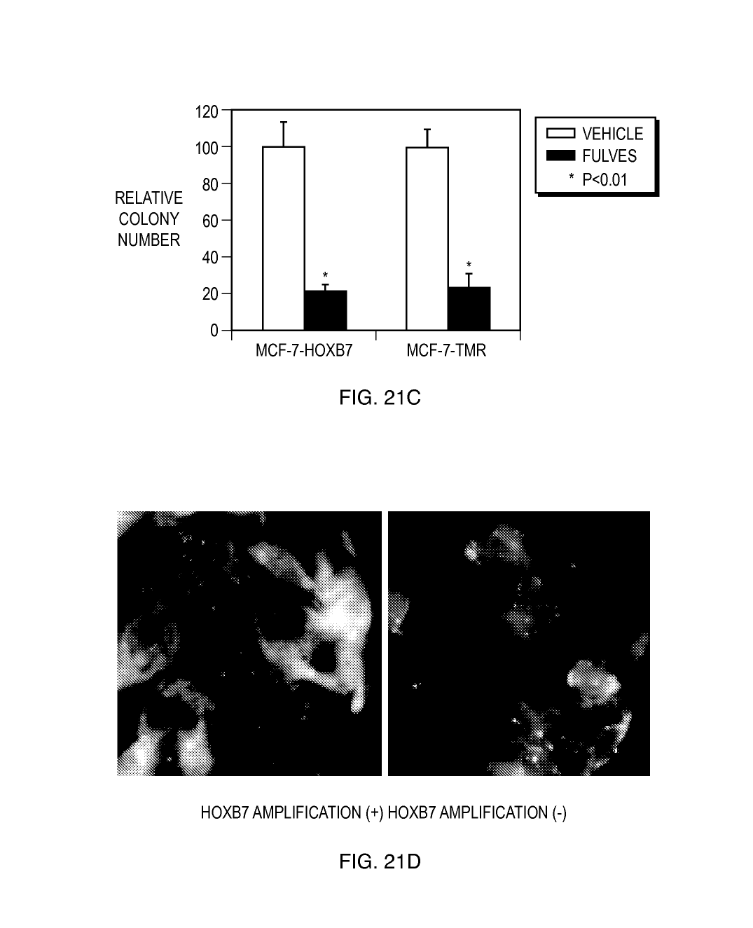

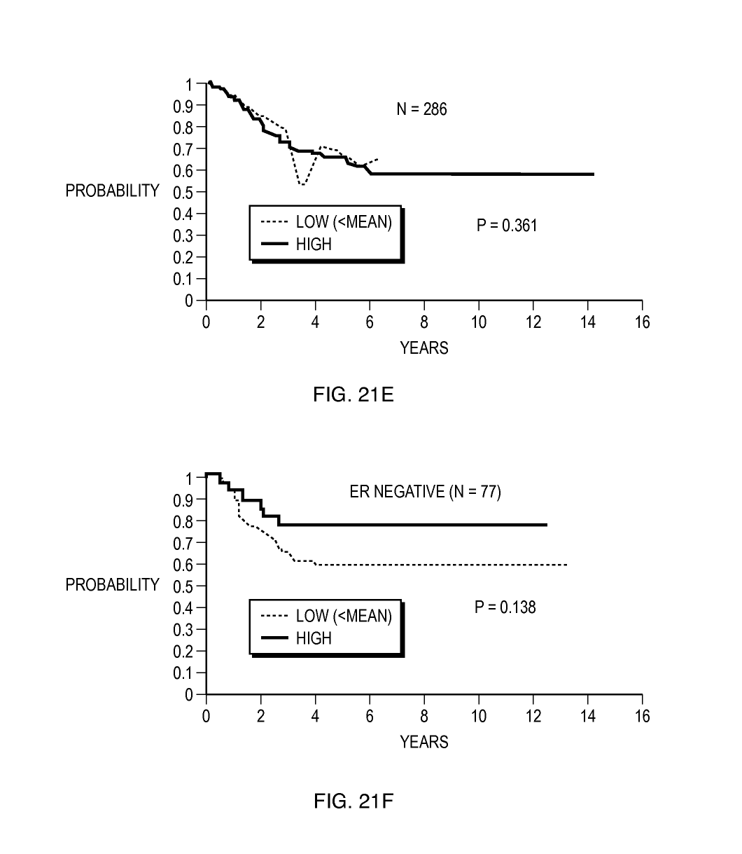

Prolonged endocrine therapy is often associated with an increase in receptor tyrosine kinase (such as EGFR/HER2, IGF-1 receptor) expression, which together with activation of ER-dependent gene transcription and aberrant growth/apoptosis leads to endocrine resistance^{16,17}. Because of the striking similarity of molecular events which are shared by prolonged endocrine therapy-primed SERM-resistant models and our HOXB7 overexpressing system, we explored the hypothesis that HOXB7 is the mediator of prolonged tamoxifen treatment induced anti-estrogen resistance. We therefore treated MCF-7 cells with either vehicle or 1 μM or 0.1 μM tamoxifen for at least 6 months (designated MCF-7-TMR1 or MCF-7-TMR2 respectively). MCF-7-TMR cells exhibited significant resistance to tamoxifen treatment as determined by colony formation and ERE-Luc activity (FIG. 21). Long-term tamoxifen treatment caused elevated expression of EGFR, HER2 and ERα. In parallel, HOXB7 expression level was also found to be potently elevated (FIG. 17a). In addition, MCF-7 cells treated over time (0, 2, 4, 6 months) with 0.1 μM tamoxifen showed progressively increasing levels of expression of HOXB7. This was accompanied by concomitant increases in expression of EGFR/HER2 and ERα (FIG. 17b) as well as other RTKs (data not shown). Remarkably, abrogation of HOXB7 expression by siRNA reversed each of the observed molecular events in the MCF-7-TMR cells (FIG. 17c). We further examined whether HOXB7 expression is tamoxifen-inducible in a second ER-positive breast cancer cell line, T47D, with low endogenous levels of HOXB7. Short-term treatment of T47D cells with 1 μM tamoxifen resulted in increased expression of HOXB7. Interestingly, expression levels of EGFR and HER2 were also induced by tamoxifen (FIG. 17d). These findings further supported our hypothesis that elevated HOXB7 expression might play an early role in the development of tamoxifen resistance. Tamoxifen stimulated expression of HOXB7 at the mRNA level dependent on ERα functionality (FIG. 17e). Further, CHIP analysis revealed that tamoxifen treatment of T47D cells promoted binding of ERα, but not ERβ, to HOXB7 promoter region (FIG. 17f).

In the clinic, nearly two-thirds of tamoxifen-resistant patients retain sensitivity to fulvestrant¹. We investigated whether this is also the case in HOXB7-overexpressing, tamoxifen-resistant MCF-7 cells. Both MCF-7-B7 and MCF-7-TMR cells remained sensitive to fulvestrant treatment in vitro (FIG. 21). We further inquired whether the pharmacological effects of fulvestrant include HOXB7 targeting. Indeed, HOXB7 levels in MCF-7-TMR cells treated with 1 μM fulvestrant for 24 h were lower compared to vehicle treated cells (FIG. 17g). Consistent with previous observations, expression of EGFR and HER2 was also significantly reduced as a result of exposure to fulvestrant in MCF-TMR cells (FIG. 17g). These results further support a causal relationship between HOXB7 overexpression and function of EGFR and HER2 in MCF-7 cells upon long-term treatment with tamoxifen. Consistent with this finding, fulvestrant reduced HOXB7-promoted colony formation in vitro in a dose-dependent manner and tumor formation in vivo (FIG. 17h). Thus, fulvestrant might be able to target tamoxifen resistant ER-positive breast cancer cells via HOXB7-dependent pathways.

Whether HOXB7 overexpression is a molecular feature shared by other anti-estrogen resistance models is an interesting question. Factors contributing to SERM-resistance have been previously studied using at least two well known model systems, an in vitro, long-term estrogen deprivation model, MCF-7-LTED^{18,19} and an in vivo, long-term tamoxifen-treated xenograft model, MCF-7-TAMLT²⁰. We therefore examined expression of HOXB7, EGFR, HER2 and ERα in cell lysates of these two systems along with our MCF-7-TMR model. We observed that HOXB7 and EGFR expression were elevated in both models (FIG. 17i). To summarize, we compared the expression levels of 4 biomarkers in four different models presented in current study. Strikingly, both HOXB7 and EGFR expression were uniformly elevated in all the four models, whereas HER2 expression was elevated in three of the four models (FIG. 17j).

Based on previous findings (ref), we investigated whether HOXB7 overexpression could be traced to gene amplification by performing CGH on a tissue microarray of xxxxxx tumors. A very low proportion of the tumors (3%) showed amplification of HOXB7 in these tumors (data not shown).

### Prognostic Significance of HOXB7 Overexpression.

To determine if HOXB7 could be utilized for the predictor of tamoxifen response, we examined HOXB7 expression levels of hormone receptor-positive primary breast cancers in a set of 60 patients treated with adjuvant tamoxifen monotherapy by real-time PCR. We found the association between a higher expression level of HOXB7 and poorer relapse-free survival is marginally significant (P=0.05) (FIG. 17k). On the other hand, we made use of the publicly available microarray dataset from Wang et al (ref) to test in silico whether overexpression of HOXB7 predicts breast cancer outcome for this group of patients who did not receive adjuvant therapy. HOXB7 expression alone does not predict clinical outcome (data not shown). However, when we analyze these data by ER status, we observe that high levels of HOXB7 predict for a worse outcome in the ER+ (p=0.04) (FIG. 17l) but not in the ER− subsets (data not shown). Thus, elevated HOXB7 expression under various scenarios appears to serve as a unifying molecular hub for the development of anti-estrogen resistance.

Herein we describe, multiple lines of evidence have been presented to support the hypothesis that a common mechanism to tamoxifen resistance is attributable to HOXB7 overexpression. HOXB7 acts via simultaneous upregulation of two receptor tyrosine kinases, EGFR and HER2, each of which was efficiently reversed by HOXB7-specific siRNA transfection. Although Hox genes have been implicated in the regulation of several pathways involved in embryogenesis and organogenesis, few target genes have been shown to be under their direct regulatory control^{5,6}. Here we showed that HOXB7 binds directly to the EGFR promoter to promote its gene transcription. The mechanisms utilized by HOXB7 to regulate the expression of HER2 and other RTKs (data not shown) are under investigation. It is interesting to observe that HOXB7 regulates the expression of multiple members of RTKs. Co-existence of a RTK proteome at elevated levels presents a formidable obstacle to successful breast cancer therapy. Many RTKs, such as EGFR/HER2 and IGF-1R, substantially contribute to anti-estrogen resistance in the breast cancer (refs). Consistently, in a small cohort of primary breast cancer tissue, we found It is hard to envision the current target-specific (usually one target, one molecule) anti-cancer therapeutics can efficiently eradicate heterogeneous populations of cells typical of breast cancer. Our data implicated HOXB7 as one of those unique targets acting upstream of many RTKs, functional antagonism of which might substantially modify the receptor tyrosine kinase proteome in breast cancer.

It is interesting to note that elevated HOXB7 expression in ER-positive but not ER-negative breast cancer patients significantly correlate with poorer relapse-free survival. It seems HOXB7 might exert a more dominant role in ER-positive cells than ER-negative cells to promote breast cancer progression. In other words, ER expression and its signaling might render the cells more dependent on HOXB7-mediated pathways such as EGFR, HER2, or ER itself as suggested in our study. At the one hand, it is fascinating to note that the preliminary data from two independent clinical trials (refs) of Iressa in breast cancer suggested that, contrary to predictions, the ER-negative breast cancer patient with high levels of EGFR expression showed poor response to Iressa; Whereas, two thirds of ER-positive patients with a low to medium level expression of EGFR overexpression showed good response to Iressa. This is which is also in line with our observation here (FIG. 15f). It is likely that ER-dependent pathways produce much more of EGFR ligands such as HB-EGF (ref), Amphiregulin (Ciarloni L, 2007) and TGFα (Saeki T, 1991), thereby render the cells more dependent on the EGFR pathway and therefore more vulnerable to Iressa attack. HOXB7 overexpression in ER-positive breast cancer might have the potential to serve as a selection marker for Iressa treatment. At the other hand, our recent data show that HOXB7 overexpression also results in elevated expression of FOXA1 (our unpublished data). In this connection, it very interesting to note that FOXA1 was reported to bind the enhancer region of ERα to promote its expression, and ER-dependent gene transcription (refs). Thus, HOXB7 might make use of coordinated actions of ER and EGFR/HER2 dependent pathway in breast tumorigenesis. Further evidence supporting such a premise was elucidated in acquired tamoxifen resistance models.

Tamoxifen resistance occurs by clonal selection of breast cancer cells that grow, paradoxically, in response to tamoxifen^{1,11,21}. It is believed that a profound change of gene expression pattern occurs during this process. MCF-7 cells treated over extended periods with tamoxifen in vitro develop tamoxifen-resistance, which is paralleled by an elevated expression of HOXB7, EGFR/HER2 and ERα (FIG. 17a,17b). We showed that these molecular events can be largely re-capitulated in MCF-7 cells by overexpression of a single gene, HOXB7, accompanied by an acquired resistance to tamoxifen. In line with this claim, abrogation of HOXB7 overexpression was sufficient to reverse most the molecular events that ensued. Thus, HOXB7 overexpression might be the hub of convergence of multiple pathways in the initiation and maintenance of both acquired and intrinsic tamoxifen resistance as illustrated in FIG. 17m.

Another striking finding was that elevated expression of HOXB7 was shared by all four different anti-estrogen resistance models (FIG. 17j). This further suggests a role for HOXB7 in the development of anti-estrogen resistance in different settings. HOXB7 siRNA transfection significantly reversed most of the malignant traits and molecular changes in both the native (BT474) and established anti-estrogen resistance models, further supporting the validity and efficiency of the HOXB7 targeting approach.

Most acquired anti-estrogen resistance models in the literature were subjected to a long-term stringent environmental stress and clonal selection^{1,2}. Molecular adaptations during resistance to either tamoxifen or estrogen deprivation utilize multiple signaling pathways, often involving cross-talk with a retained and functional estrogen receptor^{11,21,23}. It is very interesting to observe that a functional ERα is essential for elevation of HOXB7 expression upon tamoxifen treatment in this scenario. On the other hand, ER+ patients with higher levels of HOXB7 are more likely to relapse early than those with lower levels of HOXB7 whether or not they receive the adjuvant therapy with tamoxifen. Thus, the early effective antagonism of ER pathway by use of alternative therapeutic approach might benefit those ER+ patients with higher levels of HOXB7 expression. Clinically, fulvestrant was used to circumvent the acquired tamoxifen resistance by directly targeting ERα. Based on our data, we propose a novel mechanism for fulvestrant action—that it acts through targeting HOXB7 for its biological effects. HOXB7 overexpression in breast cancer, as described herein is an indicator for the selection of fulvestrant, instead of tamoxifen, as first line treatment in the clinical setting.

### REFERENCES

1. Lewis J. S. & Jordan V. C. Selective estrogen receptor modulators (SERMs): mechanisms of anticarcinogenesis and drug resistance. Mutat. Res. 591, 247-263. (2005)
2. Ali S. & Coombes R. C. Endocrine-responsive breast cancer and strategies for combating resistance. Nat. Rev. Cancer. 2, 101-112. (2002)
3. Johnston S. R. D., et al. Changes in estrogen receptor, progesterone receptor, and pS2 expression in tamoxifen-resistant human breast cancer. Cancer Res. 55, 3331-3338. (1995)
4. Gutierrez M C, et al. Molecular changes in tamoxifen-resistant breast cancer: relationship between estrogen receptor, HER-2, and p38 mitogen-activated protein kinase. J. Clin. Oncol. 10, 2469-2476. (2005)
5. Garcia-Fernandez J. The genesis and evolution of homeobox gene clusters. Nat. Rev. Genet. 6, 881-892. (2005)
6. Chen H. & Sukumar S. Role of homeobox genes in normal mammary gland development and breast tumorigenesis. J. Mammary Gland Biol. Neoplasia. 8, 159-75. (2003)
7. Care A., et al. Transduction of the SkBr3 breast carcinoma cell line with the HOXB7 gene induces bFGF expression, increases cell proliferation and reduces growth factor dependence. Oncogene. 16, 3285-3289. (1998)
8. Care A., et al. HOXB7: a key factor for tumor-associated angiogenic switch. Cancer Res. 61, 6532-6539. (2001)
9. Hyman E., et al Impact of DNA amplification on gene expression patterns in breast cancer. Cancer Res. 62, 6240-6245. (2002)
10. Wu X., et al. HOXB7, a Homeodomain Protein, is overexpressed in breast cancer and confers epithelial-mesenchymal transition. Cancer Res. 66, 9527-9534. (2006)
11. Jordan V C, et al. Changing role of the oestrogen receptor in the life and death of breast cancer cells. Breast. 12, 432-441. (2003)
12. Martin L A, et al. The anti-oestrogen ICI-182,780, but not tamoxifen, inhibits the growth of MCF-7 breast cancer cells refractory to long-term oestrogen deprivation through down-regulation of oestrogen receptor and IGF signalling. Endocr. Relat. Cancer. 12, 1017-1036. (2005)
13. Meric F, et al. Expression profile of tyrosine kinases in breast cancer. Clin. Cancer Res. 8, 361-367. (2002)
14. Hynes N. E. & Lane H. A. ERBB receptors and cancer: the complexity of targeted inhibitors. Nat. Rev. Cancer. 5, 341-354. (2005)
15. Nishi H., Nishi K. H. & Johnson A. C. Early Growth Response-1 gene mediates up-regulation of epidermal growth factor receptor expression during hypoxia. Cancer Res. 62, 827-834. (2002)
16. Nicholson R. I., et al. Growth factor signaling networks in breast cancer and resistance to endocrine agents: new therapeutic strategies. J Steroid. Biochem. Mol. Biol. 93, 257-262. (2005)
17. Osborne C K, Shou J, Massarweh S, & Schiff R. Crosstalk between estrogen receptor and growth factor receptor pathways as a cause for endocrine therapy resistance in breast cancer. Clin. Cancer Res. 11, 865s-870s. (2005)
18. Jeng M. H., et al. Estrogen receptor expression and function in long-term estrogen-deprived human breast cancer cells. Endocrinology. 139, 4164-4174. (1998)
19. Santen R J, et al. Adaptive hypersensitivity to estrogen: mechanisms and clinical relevance to aromatase inhibitor therapy in breast cancer treatment. J. Steroid Biochem. Mol Biol. 95, 155-65. (2005)
20. Osipo C., et al. Paradoxical action of fulvestrant in estradiol-induced regression of tamoxifen-stimulated breast cancer. J. Natl. Cancer Inst. 95, 1597-1608. (2003)
21. Johnston S. R., et al. Integration of signal transduction inhibitors with endocrine therapy: an approach to overcoming hormone resistance in breast cancer. Clin. Cancer Res. 9, 524S-532S. (2003)
22. Yao K, et al. Antitumor action of physiological estradiol on tamoxifen-stimulated breast tumors grown in athymic mice. Clin Cancer Res. 6, 2028-36. (2000)
23. Nicholson R. I. & Johnston S. R. Endocrine therapy—current benefits and limitations. Breast Cancer Res. Treat. 93, S3-510. (2005)
24. Shou J., Massarweh S., Osborne C. K., Wakeling A. E., Ali S., Weiss H., Schiff R. Mechanisms of Tamoxifen Resistance: Increased Estrogen Receptor-HER2/neu Cross-Talk in ER/HER2-Positive Breast Cancer Journal of the National Cancer Institute 2004 96(12):926-935.

### Example 4

### HOXB7 Transgenic Animals

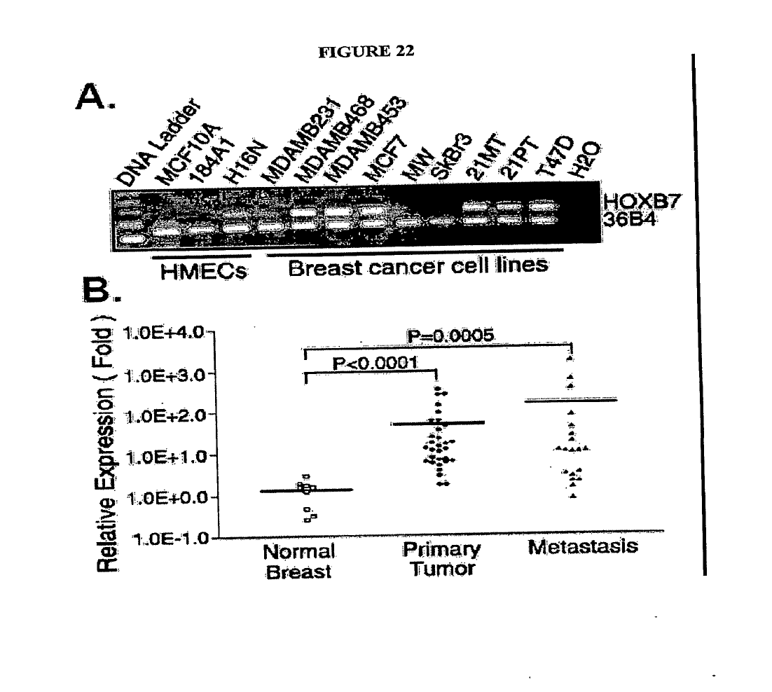

In reference to FIG. 22, expression status of HOXB7 was examined in breast cancer cell lines. It was found that HOXB7 is overexpressed in more than 60% of breast cancer cell lines. There results were further confirmed in primary carcinomas. Interestingly, the expression level of HOXB7 in metastatic tissues was further increased.

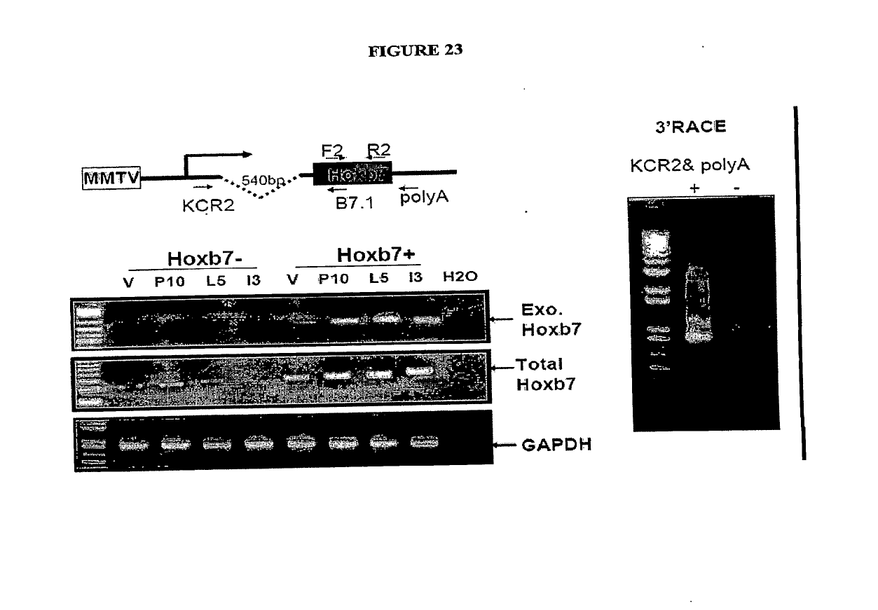

In reference to FIG. 23, to study the function of HOXB7 in vivo, a MMTV-HOXB7 transgenic mouse model was established. The full-length mouse Hoxb7 gene was inserted downstream of MMTV promoter, which drives gene expression in mammary epithelial cells. We did RT-PCR to confirm that HOXB7 was expressed at several stages of mammary gland development, virgin, pregnancy day 10, and during lactation days 5 and 13. Expression of HOXB7 is detectable in virgin mammary gland and is high during pregnancy and lactation. Since antibodies are not available western blot analysis of the mouse HOXB7, 3′RACE was done to verify that the full-length mRNA is expressed.

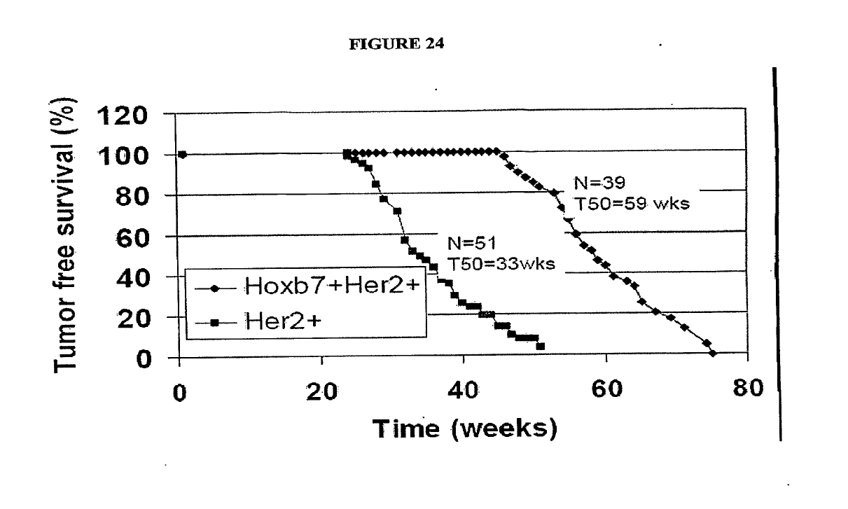

In reference to FIG. 24, HOXB7 mice did not develop mammary tumors. But in double transgenic mice that expressed HOXB7- and Her2 tumors did develop. But contrary to expectation, we found that overexpression of HOXb7 significantly delayed tumor onset and delayed the latency by about 6 months.

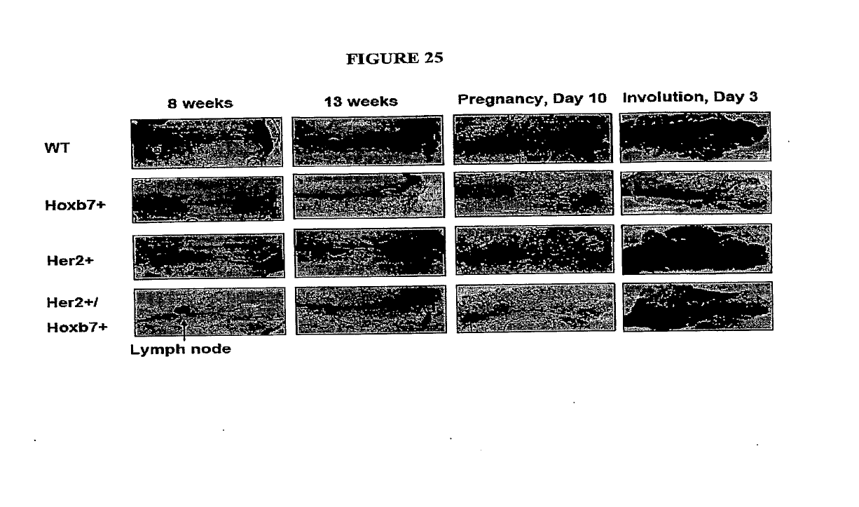

Depicted in FIG. 25, are stained whole-mounts of mammary glands which are used to grossly examine its structure during the various stages of mammary gland development. By 8 weeks, in the WT mouse the ductal tree has almost filled the fat pad. In the HOXB7 transgenic mouse, the ductal tree looks very similar to that in the WT mouse except that there is a little more dense branching. In the Her2-transgenic mouse, the ductal tree at the same time point looks different—growth is inhibited, and this has been previously described in the literature. As you can see, the ductal tree does not extend too far beyond the lymph node which is located in the center of mammary gland. Interestingly enough, in the double-transgenic mice, the phenotype in her2-transgenic mice appears to be partially rescued by HOXB7. At 13 weeks, very similar phenotypes are seen at this stage, the Her2-phenotype seems to have been completely rescued. With pregnancy, these differences disappear. At the involution stage, milk-producing epithelial cells die through apoptosis beginning at day 3 post-weaning. Her2 is known to delay involution. On the contrary, overexpression of HOXB7 dramatically accelerates the involution process. Without wishing to be bound by any particular scientific theories, HOXB7 and Her2 seem to play opposite roles during these stages of mammary gland development.

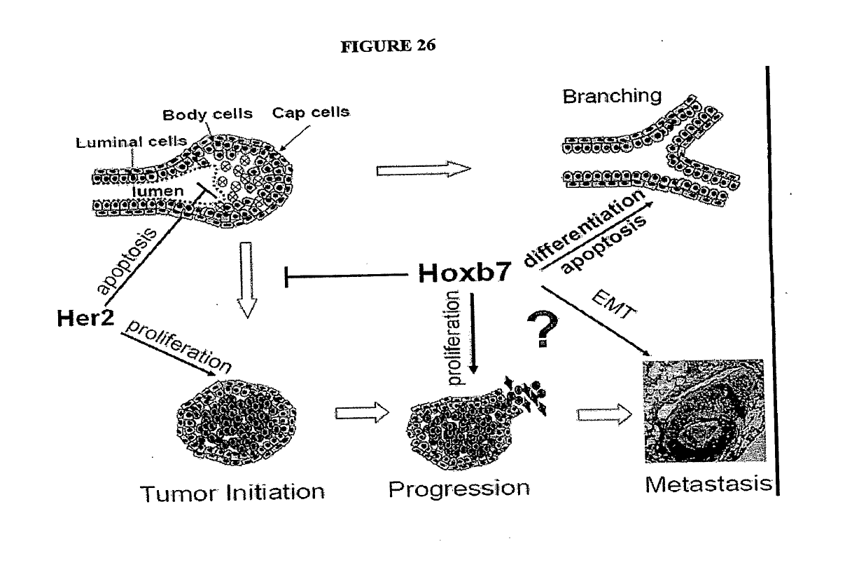

In reference to FIG. 26, to explain seemingly conflicting in vivo and in vitro data, a new model is presented herein. In this model, Her2-induced tumorigenesis was artificially divided into two phases: tumor onset and tumor progression. Overexpression of Her2 in normal mammary epithelial cells is known to inhibit cell differentiation and apoptosis, and promote cell proliferation, therefore cause tumor formation.

Proposed herein are roles of Hoxb7 in tumorigenesis is spatial and temporal. In other words, in the normal epithelial cells, over-expression of Hoxb7 may promote cellular differentiation and apoptosis, both of processes are required for normal mammary gland development such as ductal tree branching and aveloli formation. However when the tumor is onset, the tumor cells may lose the normal response to the hormone or other stimuli-induced cellular differentiation and apoptosis. At this stage, Hoxb7 may promote cellular proliferation and induce EMT as the in vitro data suggested. That Hoxb7 may promote apoptosis during normal mammary gland development is evidenced by the finding that over-expression of Hoxb7 accelerate involution process as I just showed you.

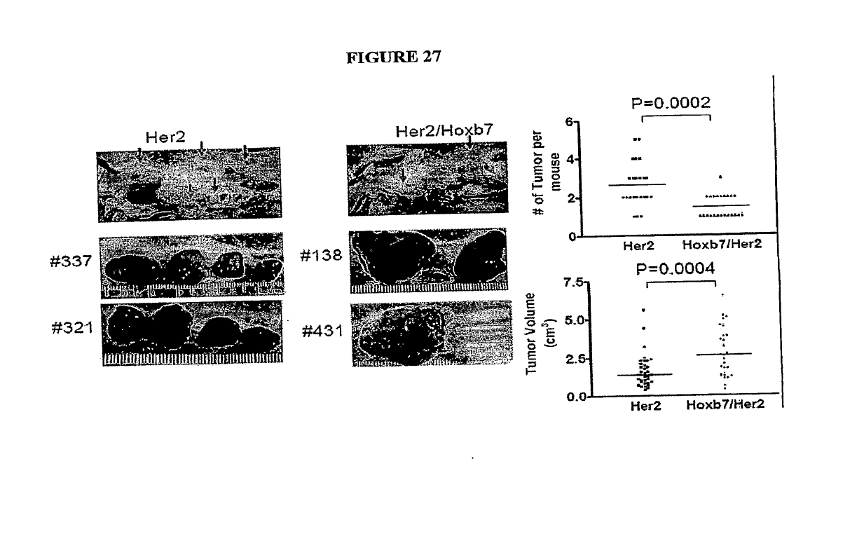

In reference to FIG. 27, to examine whether Hoxb7 promote tumor growth at the late stages of tumor progression, we sacrificed the mice at 10 weeks after we first palpated the tumor. At this time points, most of Her2-transgenic mice had multiple tumors whereas most of double transgenic mice had either one or two tumors. Overexpression of Hoxb7 significantly reduced the tumor multiplicity, which is consistent with that Hoxb7 inhibits Her2-induced tumor formation. However, when the tumor size of the first tumor on each animal was compared, it was found that although the tumor formation is delayed in double transgenic mice, the tumors in the double transgenic mice grew faster than that in the Her2-transgenic mice. That suggests that Hoxb7 indeed promote tumor growth at late stage of tumor progression.

In reference to FIG. 28, to examine whether Hoxb7 promotes cellular proliferation in vivo, we did the Ki67-staining analysis. As shown, about 30% of her2-tumor cells are ki67-positive while about 80% of Hoxb7 and Her2 double positive cells are ki67-positive. The figure to the right is the summary of ki67-staining analysis of 15 pairs of samples. These results strongly suggested that overexpression of Hoxb7 in tumor cells promotes cellular proliferation.

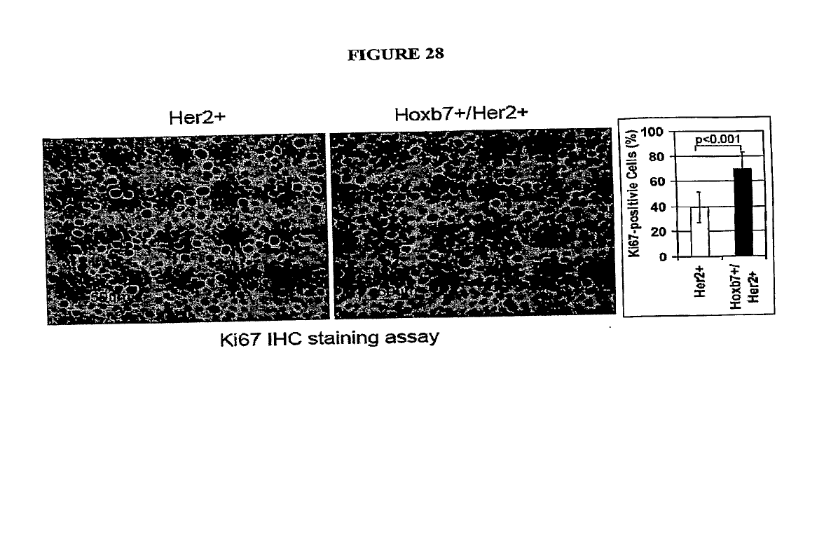

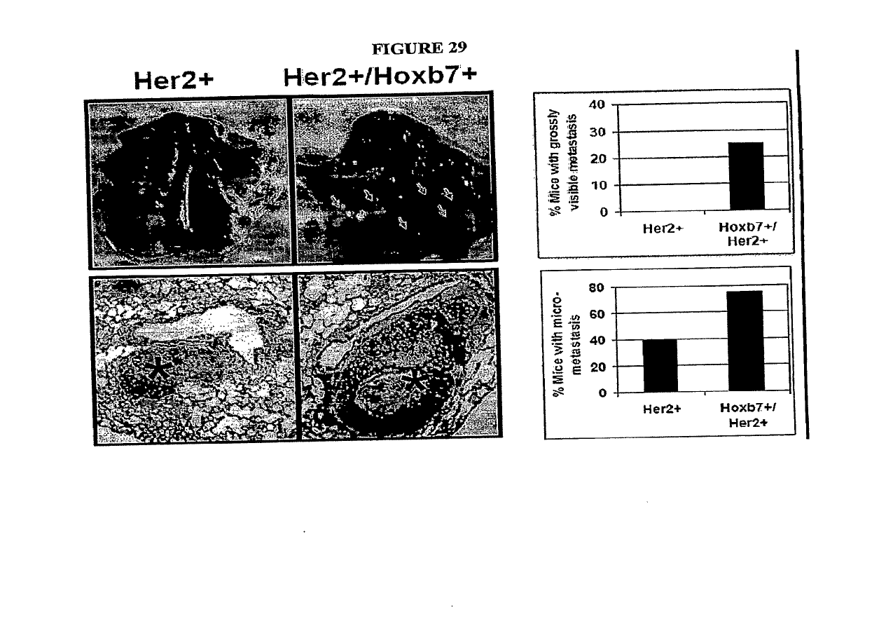

In reference to FIG. 29, the in vitro data showed that Hoxb7 induces EMT, a step for metastatic progression, it was next examined whether Hoxb7 promote tumor metastasis. The lung and liver tissue of sacrificed animals was examined. No metastatic lesion is found in liver tissue of all animal. With naked eyes, we did not find any visible metastasis in the lung of her2-transgenic mice either, but 25% of double transgenic mice developed visible lung metastasis. However, when the lung tissue was examined under the microscope, it was found that 40% of her2-transgenic mice developed micro metastasis, whereas 75% of double transgenic mice had micro metastasis. In general, the metastatic foci are larger in double transgenic mice. These data suggest that Hoxb7 promote lung metastasis of her2-induced tumor.

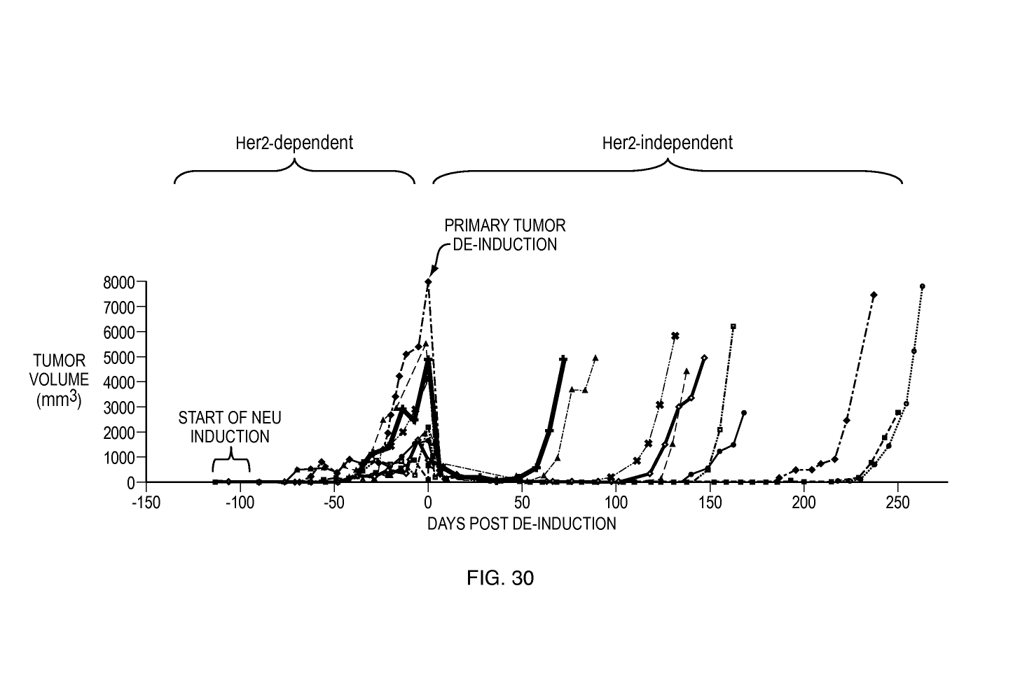

In reference to FIG. 30, the molecular basis for the dual role of Hoxb7 in Her2-induced tumorigeneis-delay tumor onset and promote tumor progression was examined. When expression of her2 is induced by doxycycline, all of the mice develop multiple tumors. Removal of the doxycycline results in tumor regression. However, following a certain period of time, most of mice develop tumor recurrences in the absence of her2 expression. More interestingly, most of these recurrent tumors are more aggressive and display characteristics of EMT.

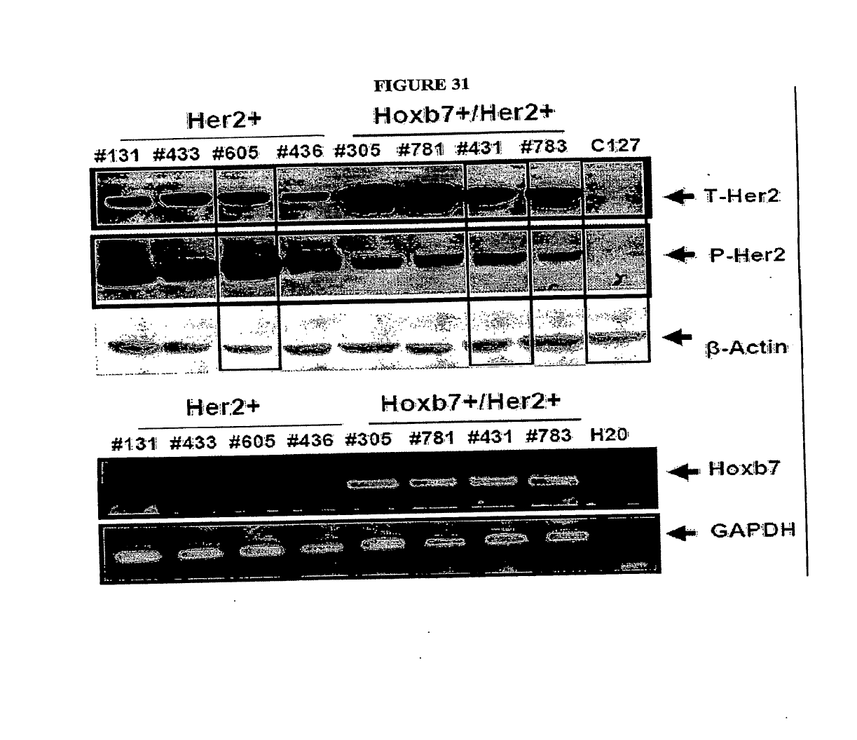

In reference to FIG. 31, to answer this question, we first examined the expression of Her2 in both Hoxb7+/− tumor samples. Although the expression levels of her2 vary from sample to sample, in general there is no significant difference in the expression level of Her2 between Hoxb7 positive and negative samples. But the phosphorylation level is significantly lower in Hoxb7/Her2 double positive cells. Hoxb7 expression can only be detected in Hoxb7-transgenic mouse tumor cells.

Next we attempt to examine the her2-signaling pathways. Since Her2 is tyrosine kinase, we decided to examine the tyrosine phosphorylome using proteomic methods. Using three cells lines (C127, normal control, #605 Her2 tumor cells and #431, double positive cells). As shown here, #605 and #431 express similar levels of Her2/neu, very low level of Her2 is detected in C127 cells.

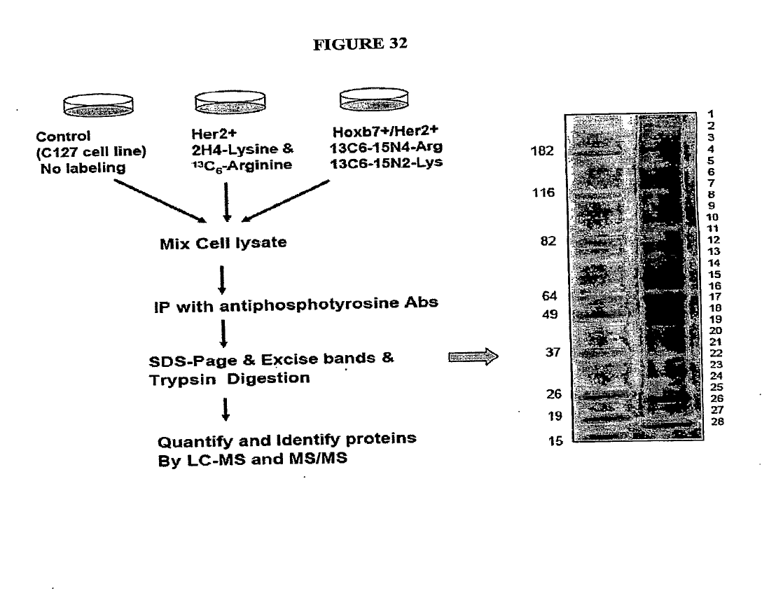

In reference to FIG. 32, cells were labeled using SILAC methods. C127, normal mouse epithelial cells without expression of Her2 and Hoxb7, serve as control and are labeled with light isotope. Cell line derived from Her2-transgenic mice was labeled with medium heavy isotope and cell lines derived from double transgenic mice were labeled with heaviest isotopes. Equal amount of cell lysates were mixed and IP with anti-phosphorylated tyrosine Abs and separated on SDS gel. The gel was cut into 40 small bands and digested with trypsin. The tryptic peptide were quantitated and identified by tandem-MS analysis.

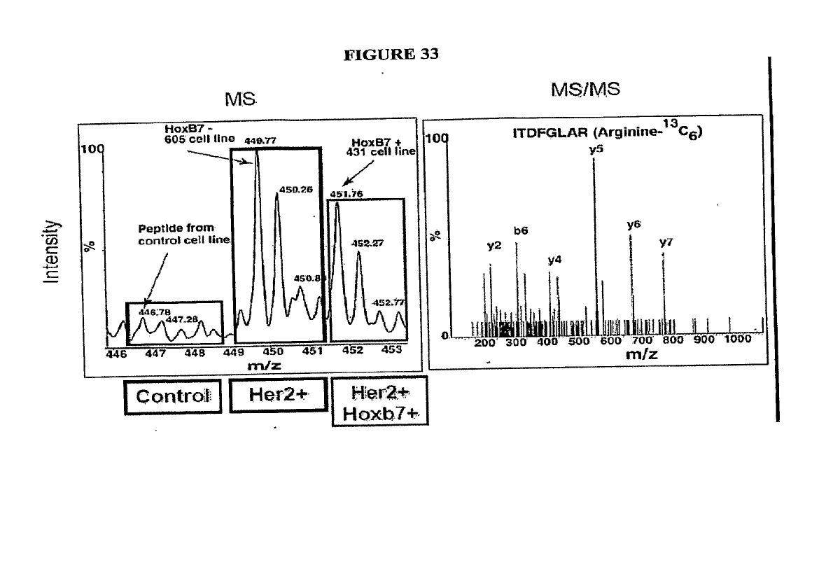

In reference to FIG. 33, a MS spectrum of Her2-derived peptide. There is only basal signal from control cells, very strong signal from Her2-cells and relative weak signal from double positive cells. Since these peptides are derived from anti-phosphorylated tyrosine Ab pulled down protein, the intensities reflects the relative amount of phosphorylated her2 protein in each type of cells. That means that there is a about 10-fold increase in phosphorylation of Her2 in her2-transgenic tumor cells, and about 1.5 fold inhibition of Her2 phosphorylation by Hoxb7 in double-transgenic tumor cells compared to Her2-transgenic tumor cells. The right panel shows the peptide sequence identified by Tandem-MS/MS.

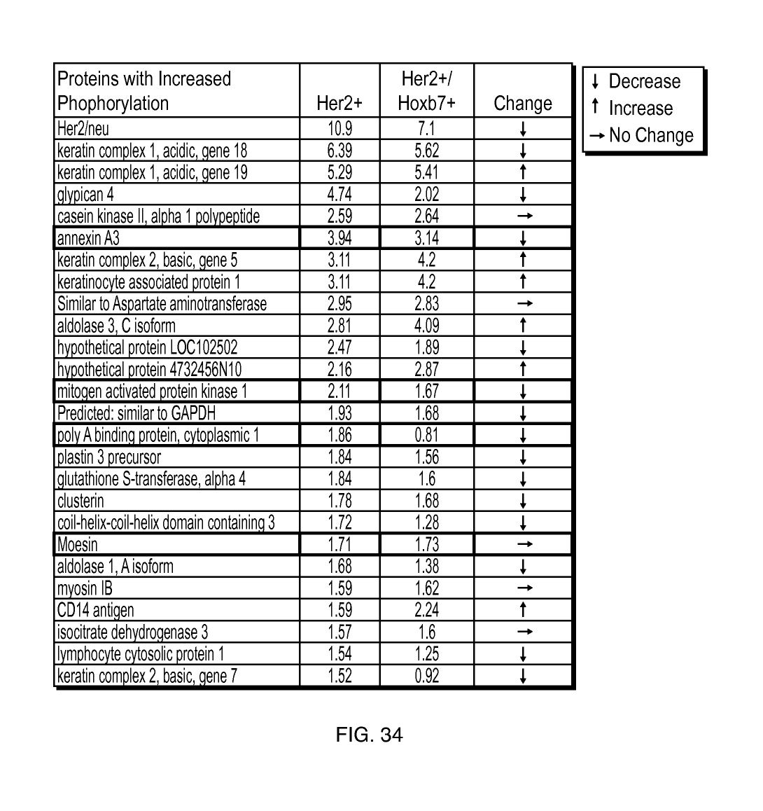

In reference to FIG. 34, by using SILAC, 395 proteins were identified and quantitated. Among these gene, 26 proteins showed significant increase in phosphorylation, and 91 proteins showed a significant decrease. Four major patterns of changes in protein phosphorylation were observed. For example, Map kinase 1 showed increased phosphorylation in Her2 tumor cells and inhibition of phosphorylation by Hoxb7 in double transgenic tumor cells. Guanine-binding protein shows a decrease in Her2-cells and increase in double-transgenic cells. The phosphorylation of Keratin 18 increased in both Her2 and Hoxb7/Her2 tumor cells, no significant changes between two cells. Another protein show decreased phosphorylation in both Her2 and Hoxb7/Her2 cells.

Since Her2 is tyrosine kinase, we focused on the protein with increased phosphorylation. Among these 26 proteins, 5 of them showed further increase in phosphorylation in Hoxb7?her2 cells. Five of them remains the same level but majority of them displayed a decreased phosphorylation in Hoxb7/Her2 cells. 4 out of these 26 proteins are known Her2 signaling proteins including MAP kinase 1, Annexin A3, poly(A)-binding protein cytoplasmic 1 (PABPC1) and Moesin. Identification of these known Her2 signaling protein provides proof of concept for this approach. Based on these data, we would like to conclude that Hoxb7 may negatively regulate Her2-signaling during Her2-induced tumorigenesis.

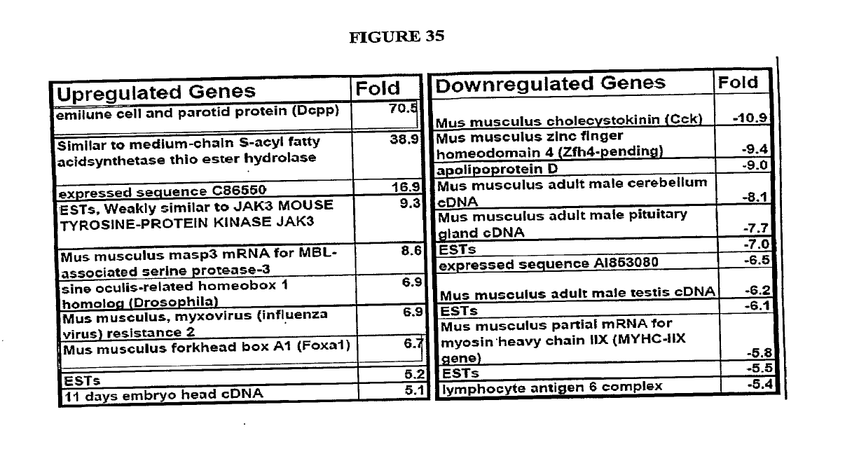

In reference to FIG. 35, since Hoxb7 is a transcriptional factor, microarray analysis was used to identify the Hoxb7 target genes. A list of genes which are upregulated and downregulated in Hoxb7-expressing cells was found. Dcpp and Foxa1 were upregulated. Dcpp which is recently identified gene one of the target gene of Estrogen receptor during development and Foxa1 is a cofactor of ER. Binding of Foxa1 to DNA can facilitate the binding to ER to ERE and therefore enhance ER-transcriptional activities. The downstream target genes include ER and Foxa1 themselves. These results imply that ER-signaling pathways may be activated by Hoxb7.

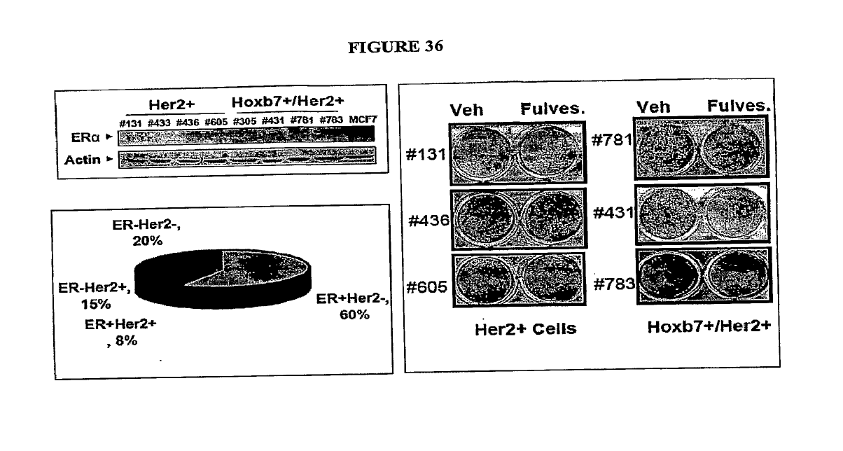

In reference to FIG. 36, Her2-carcinoma is generally considered as ER-negative tumors although very low level of ER expression can be detected. When the expression levels of ER was examined in both Hoxb7-negative and positive tumor cells, it was found that the expression level of ER is increased in Hoxb7-positive cells. Compared to MCF7 cells, the ER expression levels in the Hoxb7+ cells are still relative low. That lends us the possibility that Hoxb7 links Her2 and ER signaling pathways together.

Around 60% of breast cancers are ER-positive and 20-25% of breast cancers are Her2-positive, in most of cases, their expression are exclusive. Only very small portion (about 8%) of breast cancers are double positive and the remainder (about 20%) are double negative. The expression of level of ER in double-positive tumor is significantly lower that that in the ER+/her2− tumors. Even though they are ER-positive, they are generally resistant to Tamoxifen treatment. Some studies indicated that those double patients may benefit other anti-ER therapy like aromatase inhibitor (Letrozone) or fulvestrant. Hoxb7+Her2+ double positive cells, as shown herein are a model for these double positive breast cancer.

The sensitivities of these cells to anti-ER drugs were tested. It was found that while Her2 positive transgenic mammary tumor cells did not respond to E2, Tamoxifen or fulvestrant since they do not express ER, and Her2/Hoxb7 transgenic mouse mammary tumor cells strongly respond to fulvestrant-treatment although not responding to E2 and Tamoxifen treatment. These data are very consistent with the clinical data that her2 positive tumors generally do not respond to tamoxifen. But the fact that double transgenic mammary tumor cells respond to fulvestrant is novel and shows that human tumors overexpressing both HER2 and HOX7 may not respond to tamoxifen but will respond to fulvestrant or to aromatase inhibitors. These experiments also show that these cells provide a xenograft model in nude mice or syngeneic FVB/N mice that can be used to screen drugs and drug combinations to which these tumors respond. Nearly 10% of all human breast cancers are HER2 and HOXB7-double positive.

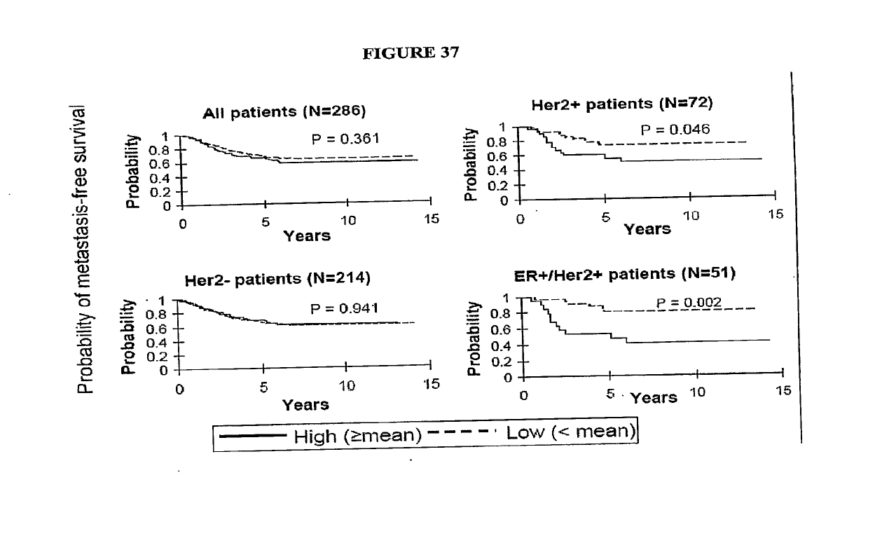

In reference to FIG. 37, to test whether overexpression of Hoxb7 has any clinical effects in breast cancer, statistical analysis was done using published microarray data. It was found that Hoxb7 alone can not predict the clinical outcome. However, when we divide the patients into ER+/ER− or Her2+/Her2− subgroups, we found that ER+ or Her2+ patient with high levels of Hoxb7 expression have a worse prognosis.

These results have a significant clinical implication. There are two possibilities to explain the poor prognosis for these Her2/Hoxb7 positive patients. First, overexpression of Hoxb7 may be associated with aggressive tumor behavior; the second possibility is that these groups of patients did not receive right treatment regimen. For these Her2+Hoxb7+ breast cancer patients, the tumor is likely to express very low level of ER if any. So it is most likely that they will either not receive hormonal treatment or be treated with tamoxifen for a short period of time and quickly develop tamoxifen resistance. So the hormonal treatment is normally not a choice for them. Our results imply that these patients should be treated with Trastuzumab followed by treatment with anti-ER reagents such as aromatase inhibitor or Fulvestrant. Considering the low level of ER expression, they may not respond to Tamoxifen.

1. A method of modulating cellular processes, comprising modulating the functional level of a HOXB7 protein.

2. (canceled)

3. A method for the treatment and/or prophylaxis of a DNA repair condition in a mammal, comprising modulating the functional level of a HOXB7 protein in the mammal, wherein increasing functional levels of the HOXB7 protein level increases DNA repair activity of a cell.

4. The method according to claim 3, wherein DNA repair is up-regulatable by HOXB7 protein over-expression.

5. The method according to claim 3, wherein the increasing functional levels of the HOXB7 protein level is by up-regulation of a HOXB7 protein level and the up-regulation comprises introducing a nucleic acid molecule encoding a HOXB7 protein or functional equivalent, derivative or homologue thereof or the HOXB7 protein expression product or functional derivative, homologue, analogue, equivalent or mimetic thereof to the cell.

6. The method of claim 3, wherein the DNA repair condition comprises one or more of xeroderma pigmentosum (XP), Cockayne syndrome (CS), trichothiodystrophy (TTD), Fanconis anemia (FA), Bloom syndrome (BS), ataxia telangiectasia (AT), Fanconi's anemia, breast cancer or colon cancer.

7. The method of claim 1, wherein decreasing the functional levels of HOXB7 protein decreases epithelial-mesenchymal transition (or cancer progression or promotes migration and invasion characteristics) of a cell.

8. A method for the treatment and/or prophylaxis of a condition characterized by aberrant or otherwise unwanted epithelial-mesenchymal transition, comprising modulating the functional level of a HOXB7 protein, wherein decreasing functional levels of HOXB7 protein decreases epithelial-mesenchymal transition.

9. A method for the treatment and/or prophylaxis of a condition characterized by estrogen-response modulator resistance, comprising modulating the functional level of a HOXB7 protein in, wherein decreasing functional levels of HOXB7 protein.

10. The method according to claim 8 or 9, wherein the modulation is down-regulation of HOXB7 protein levels and the down-regulation comprises contacting the cell with a compound that functions as an antagonist to the HOXB7 protein expression product.

11. The method of claim 10, wherein the estrogen-response modulator resistance comprises tamoxifen resistance.

12. The method according to claim 1, wherein the modulation comprises contacting the cell with a compound that modulates transcriptional and/or translational regulation of a HOXB7 gene.

13. The method of claim 12, wherein the compound comprises an siRNA targeting HOXB7.

14. The method of claim 14, wherein the siRNA comprises one or more of 5′-ATATCCAGCCTCAAGTTCG-3′ or 5′-ACTTCTTGTGCGTTTGCTT-3′.

15-16. (canceled)

17. A pharmaceutical composition comprising a pharmaceutically effective amount of a HOXB7 modulator effective to treat, prevent, ameliorate, reduce or alleviate a HOXB7 related disorder or symptoms thereof and a pharmaceutically acceptable excipient.

18. The pharmaceutical composition of claim 17, wherein the HOXB7 modulator is selected from one or more of a small molecule, RNAi molecule, an anti-HOXB7 antibody, an antigen-binding fragment of an anti-HOXB7 antibody, a polypeptide, a peptidomimetic, a nucleic acid encoding a peptide, or an organic molecule.

19. A method to treat, prevent, ameliorate, reduce or alleviate a HOXB7 related disorder or symptoms thereof, comprising: administering to a subject in need thereof a composition comprising a pharmaceutically effective amount of a HOXB7 modulator.

20. The method of claim 19, wherein the HOXB7 modulator is one or more of a small molecule, an anti-HOXB7 antibody, an RNAi, an antigen-binding fragment of an anti-HOXB7 antibody, a polypeptide, a peptidomimetic, a nucleic acid encoding a peptide, or an organic molecule.

21. The method of claim 19, wherein the HOXB7 modulator is administered prophylactically to a subject at risk of being afflicted a HOXB7 related disorder.

22. The method of claim 19, wherein the composition further comprises a therapeutically effective amount of one or more of at least one anticonvulsant, non-narcotic analgesic, non-steroidal anti-inflammatory drug, antidepressant, glutamate receptor antagonist, nicotinic receptor antagonist, or local anesthetic.

23-25. (canceled)

26. The method of claim 19, wherein a HOXB7 related disorder or symptom thereof is indicated by alleviation of pain, progression of cancer, decreased cell proliferation, increased cell DNA repair efficiency, or an inhibition of cell proliferation.

27. The method of claim 19, further comprising obtaining the HOXB7 modulator.

28-34. (canceled)

35. A kit comprising: a) an HOXB7 modulator and a pharmaceutically acceptable carrier and b) instructions for use.

36. A transgenic non-human animal comprising an over-expressed HOXB7 protein or a fragment or variant thereof.

37-48. (canceled)

49. A method for determining treatment of a subject suffering from breast cancer, comprising, determining the level of HOXB7 expression in a tumor of the subject and correlating HOXB7 over expression an indicator for the selection of fulvestrant as treatment.

50. The method of claim 49, wherein HOXB7 negative cells are correlated with not responding to E2, Tamoxifen or fulvestrant.

51. The method of claim 49, further comprising determining the Her2 status of the tumor.

52. The method of claim 49, wherein Her2+Hoxb7+ tumors are correlated with Trastuzumab.

53. The method of claim 52, wherein treatment with Trastuzumab is followed by treatment with an anti-ER reagent.

54. The method of claim 53, wherein the anti-ER reagent comprises an aromatase inhibitor or Fulvestrant.

55. A method of determining prognosis of breast cancer, comprising: determining one or more of the HOXB7 status, ER status and Her2 status of a sample and correlating ER+ or Her2+ patient with high levels of Hoxb7 expression having a lower prognosis.


SEQUENCE LISTING (24 sequences)

SEQ ID NO: 1 - DNA, 19 bp, Artificial Sequence
  Description of Artificial Sequence Synthetic oligonucleotide
atatccagcctcaagttcg

SEQ ID NO: 2 - DNA, 19 bp, Artificial Sequence
  Description of Artificial Sequence Synthetic oligonucleotide
acttcttgtgcgtttgctt

SEQ ID NO: 3 - DNA, 19 bp, Artificial Sequence
  Description of Artificial Sequence Synthetic primer
agagtaacttccggatcta

SEQ ID NO: 4 - DNA, 19 bp, Artificial Sequence
  Description of Artificial Sequence Synthetic primer
tcggcttcagccctgtctt

SEQ ID NO: 5 - DNA, 22 bp, Artificial Sequence
  Description of Artificial Sequence Synthetic primer
tcaaaggagtgtgtgctaaccg

SEQ ID NO: 6 - DNA, 20 bp, Artificial Sequence
  Description of Artificial Sequence Synthetic primer
ctgcccagttcgtttcagtg

SEQ ID NO: 7 - DNA, 22 bp, Artificial Sequence
  Description of Artificial Sequence Synthetic primer
gattggctacccaactgttgca

SEQ ID NO: 8 - DNA, 22 bp, Artificial Sequence
  Description of Artificial Sequence Synthetic primer
caggggcagcagccacaaaggc

SEQ ID NO: 9 - DNA, 18 bp, Artificial Sequence
  Description of Artificial Sequence Synthetic primer
caggtagcgattgtagtg

SEQ ID NO: 10 - DNA, 20 bp, Artificial Sequence
  Description of Artificial Sequence Synthetic primer
acccctggatgcgaagctca

SEQ ID NO: 11 - DNA, 25 bp, Artificial Sequence
  Description of Artificial Sequence Synthetic primer
atgagttcattgtattatgcgaatg

SEQ ID NO: 12 - DNA, 27 bp, Artificial Sequence
  Description of Artificial Sequence Synthetic primer
actcttcctcttcctcctctgcttcag

SEQ ID NO: 13 - RNA, 19 nt, Artificial Sequence
  Description of Artificial Sequence Synthetic oligonucleotide
cauucuguguguaucuaaa

SEQ ID NO: 14 - RNA, 19 nt, Artificial Sequence
  Description of Artificial Sequence Synthetic oligonucleotide
uuuagauacacacagaaug

SEQ ID NO: 15 - RNA, 19 nt, Artificial Sequence
  Description of Artificial Sequence Synthetic oligonucleotide
ggacucuccuucuguaaua

SEQ ID NO: 16 - RNA, 19 nt, Artificial Sequence
  Description of Artificial Sequence Synthetic oligonucleotide
uauuacagaauuagagucc

SEQ ID NO: 17 - protein, 17 aa, Homo sapiens
VRSGNKAAVVLCMDVGF

SEQ ID NO: 18 - DNA, 20 bp, Artificial Sequence
  Description of Artificial Sequence Synthetic primer
attatccgacgctggctcta

SEQ ID NO: 19 - DNA, 20 bp, Artificial Sequence
  Description of Artificial Sequence Synthetic primer
cgggtgccctgaggagttaa

SEQ ID NO: 20 - DNA, 20 bp, Artificial Sequence
  Description of Artificial Sequence Synthetic primer
ttggctcgacctggacatag

SEQ ID NO: 21 - DNA, 19 bp, Artificial Sequence
  Description of Artificial Sequence Synthetic primer
gagggaggagaaccagcag

SEQ ID NO: 22 - DNA, 21 bp, Artificial Sequence
  Description of Artificial Sequence Synthetic primer
gcccctctcggaaattaactc

SEQ ID NO: 23 - DNA, 20 bp, Artificial Sequence
  Description of Artificial Sequence Synthetic primer
aggagcagaggaggaggaga

SEQ ID NO: 24 - protein, 8 aa, Artificial Sequence
  Description of Artificial Sequence Synthetic peptide
ITDFGLAR